(19) United States
(12) Patent Application Publication
(10) Pub. No.: US 2020/0237742 A1
(43) Pub. Date: Jul. 30, 2020

(54) Serdexmethylphenidate Conjugates, Compositions And Methods Of Use Thereof

(71) Applicant: KemPharm, Inc., Celebration, FL (US)

(72) Inventors: Sven Guenther, Coralville, IA (US); Guochen Chi, Coralville, IA (US); Travis Mickle, Kissimmee, FL (US)

(21) Appl. No.: 16/803,714

(22) Filed: Feb. 27, 2020

Related U.S. Application Data
(63) Continuation-in-part of application No. 16/433,538, filed on Jun. 6, 2019.
(60) Provisional application No. 62/828,056, filed on Apr. 2, 2019.
(60) Provisional application No. 62/814,802, filed on Mar. 6, 2019.
(60) Provisional application No. 62/768,457, filed on Nov. 16, 2018.
(60) Provisional application No. 62/744,528, filed on Oct. 11, 2018.
(60) Provisional application No. 62/731,574, filed on Sep. 14, 2018.
(60) Provisional application No. 62/729,155, filed on Sep. 10, 2018.
(60) Provisional application No. 62/695,134, filed on Jul. 8, 2018.
(60) Provisional application No. 62/685,899, filed on Jun. 15, 2018.

(51) Int. Cl. A61K 31/4545; A61K 47/46; A61K 9/48

(52) U.S. Cl. CPC A61K 31/4545; A61K 9/48; A61K 47/46

(57) ABSTRACT

The present technology is directed to one or more compositions comprising serdexmethylphenidate conjugates and unconjugated d-methylphenidate and/or a pharmaceutically acceptable salt thereof. The present technology also relates to one or more compositions and oral formulations comprising serdexmethylphenidate conjugates and unconjugated d-methylphenidate and/or a pharmaceutically acceptable salt thereof. The present technology also relates to one or more methods of using compositions comprising serdexmethylphenidate conjugates and unconjugated d-methylphenidate and/or a pharmaceutically acceptable salt thereof. The present technology additionally relates to one or more pharmaceutical kits containing a composition comprising serdexmethylphenidate conjugates and unconjugated d-methylphenidate and/or a pharmaceutically acceptable salt thereof.

### RELATED APPLICATIONS

This application is a continuation-in-part of U.S. application Ser. No. 16/433,538, Filed Jun. 6, 2019 and claims priority to and benefit from U.S. Provisional Application Nos. 62/685,899, filed Jun. 15, 2018, 62/695,134, filed Jul. 8, 2018, 62/729,155, filed Sep. 10, 2018, 62/731,574, filed Sep. 14, 2018, 62/744,528, filed Oct. 11, 2018, 62/768,457, filed Nov. 16, 2018, 62/814,802, filed Mar. 6, 2019, and 62/828,056, filed Apr. 2, 2019, each of which is incorporated by referenced in its/their entirety. The present application is also related to PCT Application Nos. PCT/US2017/65481 and PCT/US2017/65482, each of which is incorporated by reference in its/their entirety.

### FEDERALLY SPONSORED RESEARCH OR DEVELOPMENT

[Not Applicable]

### BACKGROUND OF THE INVENTION

Methylphenidate is a psychostimulant which is a chain substituted amphetamine derivative. Similar to amphetamine and cocaine, methylphenidate targets the central nervous system, specifically the dopamine transporter (DAT) and norepinephrine transporter (NET).

Stimulants, including methylphenidate (“MPH”), are believed to enhance the activity of the sympathetic nervous system and/or central nervous system (CNS). Stimulants such as MPH and the various forms and derivatives thereof are used for the treatment of a range of conditions and disorders predominantly encompassing, for example, attention deficit hyperactivity disorder (ADHD), attention deficit disorder (ADD), obesity, narcolepsy, appetite suppression, depression, anxiety and/or wakefulness.

Methylphenidate is currently approved by the United States Food and Drug Administration (“FDA”) for the treatment of attention-deficit hyperactivity disorder and narcolepsy. In some embodiments, compositions of the present technology may be administered for the treatment of attention-deficit hyperactivity disorder and narcolepsy, or any condition that requires the blocking of the norepinephrine and/or dopamine transporters.

Attention deficit hyperactivity disorder (ADHD) in children has been treated with stimulants for many years. However, more recently, an increase in the number of prescriptions for ADHD therapy in the adult population has, at times, outperformed the growth of the pediatric market. Although there are various drugs currently in use for the treatment of ADHD, including some stimulants and some non-stimulant drugs, methylphenidate (commercially available from, for example, Novartis International AG (located in Basel, Switzerland) under the trademark Ritalin®) is commonly prescribed. Moreover, during classroom trials, non-stimulants have shown to be less effective in improving behavior and attention of ADHD afflicted children than amphetamine derivatives.

Behavioral deterioration (rebound or “crashing”) is observed in a significant portion of children with ADHD as the medication wears off, typically in the afternoon or early evening. Rebound symptoms include, for example, irritability, crankiness, hyperactivity worse than in the unmedicated state, sadness, crying, and in rare cases psychotic episodes. The symptoms may subside quickly or last several hours. Some patients may experience rebound/crashing so severe that treatment must be discontinued. Rebound/crashing effects can also give rise to addictive behavior by enticing patients to administer additional doses of stimulant with the intent to prevent anticipated rebound/crashing negative outcomes and side effects.

Stimulants, such as methylphenidate and amphetamine, have been shown in the conventional art to exhibit noradrenergic and dopaminergic effects that can lead to cardiovascular events comprising, for example, increased heart rate, hypertension, palpitations, tachycardia and in isolated cases cardiomyopathy, stroke, myocardial infarction and/or sudden death. Consequently, currently available stimulants expose patients with pre-existing structural cardiac abnormalities or other severe cardiac indications to even greater health risks and are frequently not used or used with caution in this patient population.

Methylphenidate, like other stimulants and amphetamine derivatives, can become addictive and is prone to substance abuse. Oral abuse has been reported, and euphoria can also be achieved through intranasal and intravenous administration.

There is a need in the art for forms or compositions of methylphenidate that maintain the pharmacological benefit when administered, in particular via the oral route, but which preferably have no or a substantially decreased pharmacological activity when administered through injection or intranasal routes of administration. Methylphenidate is known to have several adverse effects such as fast heartbeat, chest pain, fever, joint pain, skin rash or hives. Other side effects include insomnia, nausea, headache, vomiting, decreased appetite, xerostomia, anxiety, tics, hyperhidrosis, and irritability. As such, there is also a need in the art for forms of methylphenidate or salt thereof that can minimize, reduce, or slow the onset of adverse effects when administered.

There is also a need for forms or compositions of methylphenidate that can provide improved behavior and attention of ADHD afflicted children

There is a further need in the art for forms or compositions of methylphenidate that can provide flexibility in dosing regimens. For example, a single daily dose form of methylphenidate that can provide an extended release PK profile, or that can provide both immediate and extended release PK profiles would be highly desirable.

There is yet a further need for forms or compositions of methylphenidate that can provide an early onset of efficacy, for example as soon as about 30 minutes to about 1 hour post-dosing, and/or a duration of efficacy, for example as long as about 10-13 hours.

There is also a further need for forms of compositions of methylphenidate that can provide an early onset of efficacy in human or animal patients with central nervous system disorders or conditions, such as ADHD, among others.

### BRIEF SUMMARY OF THE INVENTION

In at least one aspect, the present technology provides at least one composition comprising a serdexmethylphenidate conjugate having the following structure:

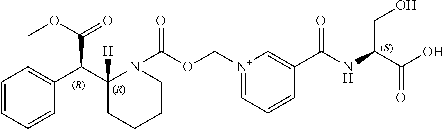

or a pharmaceutically acceptable salt thereof and following administration of the composition, each of at least the Cₘₐₓ, AUCₗₐₛₜ, or AUC_{inf} of d-methylphenidate active released from the composition is dose-proportional across at least about a 1.5-fold dose range or higher. In another aspect, following administration of the composition of the present technology each of the Cₘₐₓ, AUCₗₐₛₜ, and/or AUC_{inf} is dose-proportional across at least about a 5-fold dose range or higher. In another aspect, following administration of the composition of the present technology each of the Cₘₐₓ, AUCₗₐₛₜ, and/or AUC_{inf} is dose-proportional across at least about a 10-fold dose range or higher. In another aspect, following administration of the composition of the present technology each of the AUCₗₐₛₜ and/or AUC_{inf} is dose-proportional across at least about a 15-fold dose range or higher. In another aspect, following administration of the composition of the present technology, AUC_{inf} is dose-proportional across at least about a 25-fold dose range or higher. In another aspect, following administration of the composition of the present technology, AUC_{inf} is dose-proportional across at least about a 50-fold dose range or higher. In another aspect, following administration of the composition of the present technology, AUC_{inf} is dose-proportional across at least about a 100-fold dose range or higher. In yet another aspect, following administration of the composition of the present technology Cₘₐₓ, AUCₗₐₛₜ, and/or AUC_{inf} of d-methylphenidate active from the composition is dose-proportional across about a 6-fold, about a 11-fold, and/or about a 82-fold dose range, respectively.

In another aspect, the serdexmethylphenidate conjugate of the present technology is present in the composition in an amount that is molar equivalent to a dose of d-methylphenidate in the range of about 0.1 mg to about 1100 mg per dose, preferably in the range of about 0.1 to about 500 mg per dose, preferably in the range of about 500 mg to about 1100 mg per dose, preferably in the range of about 200 mg to about 1100 mg per dose, preferably in the range of about 300 mg to about 1050 mg per dose, preferably in the range of about 400 mg to about 1000 mg per dose, preferably in the range of about 500 mg to about 1000 mg per dose, preferably in the range of about 0.5 mg to about 480 mg per dose, preferably in the range of about 1 mg to about 250 mg per dose, preferably in the range of about 2 mg to about 240 mg per dose, preferably in the range of about 5 mg to about 200 mg per dose, preferably in the range of about 10 mg to about 150 mg per dose, preferably in the range of about 20 mg to about 100 mg per dose, preferably in the range of about 30 mg to about 80 mg per dose, or preferably in the range of about 40 mg to about 70 mg per dose.

In another aspect, the composition of the present technology is administered via oral, intravenous, intranasal, or transdermal administration. In yet another aspect, the composition is a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, or a suspension dosage form.

In another aspect, the serdexmethylphenidate conjugate of the present technology exhibits an improved AUC and rate of release over time when compared to unconjugated d-methylphenidate over the same time period. In yet another aspect, the serdexmethylphenidate conjugate of the present technology exhibits less variability in the PK profile when compared to unconjugated d-methylphenidate. In another aspect, serdexmethylphenidate conjugate of the present technology has reduced adverse effects when compared with unconjugated d-methylphenidate.

In another aspect, the serdexmethylphenidate conjugate of the present technology is provided in an amount sufficient to provide a therapeutically effective AUC of d-methylphenidate. In another aspect, the serdexmethylphenidate conjugate of the present technology is provided in an amount sufficient to provide a lower AUC and/or lower Cₘₐₓ of d-methylphenidate but similar therapeutic effect when compared to an equivalent molar amount of unconjugated d-methylphenidate. In another aspect, the serdexmethylphenidate conjugate of the present technology is provided in an amount sufficient to provide a therapeutically equivalent AUC and/or Cₘₐₓ when compared to an equivalent molar amount of unconjugated d-methylphenidate. In yet another aspect, the serdexmethylphenidate conjugate of the present technology is provided in an amount sufficient to provide a therapeutically equivalent AUC and/or a lower Cₘₐₓ when compared to an equivalent molar amount of unconjugated d-methylphenidate. In another aspect, the unconjugated d-methylphenidate comprises d-methylphenidate.

In at least one aspect, the present technology provides at least one composition comprising: (a) unconjugated d-methylphenidate, wherein the unconjugated d-methylphenidate comprises d-methylphenidate, and (b) a serdexmethylphenidate conjugate having the following chemical formula:

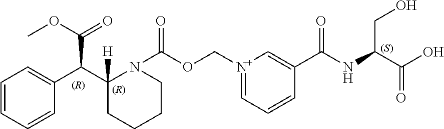

where the unconjugated d-methylphenidate and the serdexmethylphenidate conjugate is each present in the composition in an amount that is molar equivalent to a dose of d-methylphenidate in the range of about 0.1 mg to about 300 mg, and each of the Cₘₐₓ, AUCₗₐₛₜ, and/or AUC_{inf} of d-methylphenidate active from the composition is dose-proportional across at least a 1.5 fold-dose range. In another aspect, following administration of the composition of the present technology each of the Cₘₐₓ, AUCₗₐₛₜ, and/or AUC_{inf} is dose-proportional across at least a 5-fold dose range. In another aspect, following administration of the composition of the present technology each of the Cₘₐₓ, AUCₗₐₛₜ, and/or AUC_{inf} is dose-proportional across at least a 10-fold dose range. In another aspect, following administration of the composition of the present technology each of the AUCₗₐₛₜ and/or AUC_{inf} is dose-proportional across at least a 15-fold dose range. In another aspect, following administration of the composition of the present technology AUC_{inf} is dose-proportional across at least a 25-fold dose range. In another aspect, following administration of the composition of the present technology AUC_{inf} is dose-proportional across at least a 50-fold dose range. In another aspect, following administration of the composition of the present technology AUC_{inf} is dose-proportional across at least a 100-fold dose range. In yet another aspect, following administration of the composition of the present technology at least Cₘₐₓ, AUCₗₐₛₜ, and/or AUC_{inf} of d-methylphenidate active from the composition is dose-proportional across a 6-fold, 11-fold, and 82-fold dose range, respectively.

In another aspect, the serdexmethylphenidate conjugate of the present technology is present in the composition in an amount that is molar equivalent to a dose of d-methylphenidate in the range of about 0.1 mg to about 500 mg per day, alternatively in the range of about 0.5 mg to about 480 mg per day, alternatively in the range of about 1 mg to about 250 mg per day, alternatively in the range of about 2 mg to about 240 mg per day, alternatively in the range of about 5 mg to about 200 mg per day, alternatively in the range of about 10 mg to about 150 mg per day, alternatively in the range of about 20 mg to about 100 mg per day, alternatively in the range of about 30 mg to about 80 mg per day, or alternatively in the range of about 40 mg to about 70 mg per day.

In another aspect, the unconjugated d-methylphenidate of the present technology contributes a molar dose amount to the composition in the range of about 5% to about 95% relative to the total combined total molar dose of the unconjugated d-methylphenidate and the serdexmethylphenidate conjugate, alternatively about 10% to about 90%, alternatively about 20% to about 80%, alternatively about 25% to about 75%, alternatively about 30% to about 70%, alternatively about 40% to about 60%, or alternatively about 50% relative to the total combined total molar dose of the unconjugated d-methylphenidate and the serdexmethylphenidate conjugate.

In another aspect, the serdexmethylphenidate conjugate of the present technology contributes a molar dose amount to the composition in the range of about 95% to about 5%, relative to the total combined molar dose of the unconjugated d-methylphenidate and the serdexmethylphenidate conjugate, alternatively about 90% to about 10%, alternatively about 80% to about 20%, alternatively about 75% to about 25%, alternatively about 70% to about 30%, alternatively about 60% to about 40%, or alternatively about 50% relative to the total combined molar dose of the unconjugated d-methylphenidate and the serdexmethylphenidate conjugate.

In another aspect, the composition of the present technology wherein the composition has a dosing regimen of at least once a week, alternatively one time a day, alternatively about two times a day, alternatively about three times a day, or alternatively about four times a day or more. In another aspect, the composition of the present technology has a dosing regimen of every other day. In yet another aspect, the every other day dosing regimen is used in a method for the treatment of binge eating disorder.

In another aspect, the serdexmethylphenidate conjugate of the present technology is present in the composition in an amount that is molar equivalent to a dose of d-methylphenidate in the range of about 0.1 mg to about 500 mg per day, alternatively in the range of about 0.5 mg to about 480 mg per day, alternatively in the range of about 1 mg to about 250 mg per day, alternatively in the range of about 2 mg to about 240 mg per day, alternatively in the range of about 5 mg to about 200 mg per day, alternatively in the range of about 10 mg to about 150 mg per day, alternatively in the range of about 20 mg to about 100 mg per day, alternatively in the range of about 30 mg to about 80 mg per day, or alternatively in the range of about 40 mg to about 70 mg per day.

In another aspect, the total molar dose of unconjugated d-methylphenidate and serdexmethylphenidate in the composition comprises about 90% serdexmethylphenidate, alternatively about 80% serdexmethylphenidate, alternatively about 75% serdexmethylphenidate, alternatively about 70% serdexmethylphenidate, alternatively about 60% serdexmethylphenidate, or alternatively about 50% serdexmethylphenidate.

In another aspect of the present technology, the total molar dose of unconjugated d-methylphenidate and serdexmethylphenidate in the composition comprises about 10% unconjugated d-methylphenidate, alternatively about 20% unconjugated d-methylphenidate, alternatively about 30% unconjugated d-methylphenidate, alternatively about 40% unconjugated d-methylphenidate, or alternatively about 50% unconjugated d-methylphenidate.

In another aspect of the present technology, the composition comprises a salt of d-methylphenidate and a salt of serdexmethylphenidate.

In another aspect of the present technology, the composition has a dose mixture of about 1 mg to about 20 mg d-methylphenidate hydrochloride and about 20 mg to about 80 mg serdexmethylphenidate chloride, alternatively about 6 mg d-methylphenidate hydrochloride and about 28 mg serdexmethylphenidate chloride, alternatively about 9 mg d-methylphenidate hydrochloride and about 42 mg serdexmethylphenidate chloride, alternatively about 8 mg d-methylphenidate hydrochloride and about 64 mg serdexmethylphenidate chloride, alternatively about 12 mg d-methylphenidate hydrochloride and about 56 mg serdexmethylphenidate chloride, or alternatively about 16 mg d-methylphenidate hydrochloride and about 48 mg serdexmethylphenidate chloride.

In at least one aspect, the present technology provides at least one composition comprising a serdexmethylphenidate conjugate having the following chemical formula:

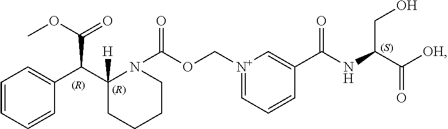

or a pharmaceutically acceptable salt thereof, wherein the composition results in minimized, reduced and/or slower onset of adverse effects after administration to a human or animal subject when compared to an equivalent molar amount of administered unconjugated d-methylphenidate.

In another aspect, the composition prevents at least one methylphenidate-related adverse effect after oral, intranasal, and/or intravenous administration to a human or animal subject when compared to an equivalent molar amount of administered unconjugated d-methylphenidate.

In another aspect, the pharmaceutically acceptable salt of the serdexmethylphenidate conjugate is serdexmethylphenidate chloride.

In another aspect, the composition further comprises unconjugated d-methylphenidate, wherein the unconjugated d-methylphenidate comprises a pharmaceutically acceptable salt of d-methylphenidate. In yet another aspect, the pharmaceutically acceptable salt of d-methylphenidate is d-methylphenidate hydrochloride.

In another aspect, the composition provides a lower AUC and/or Cₘₐₓ for d-methylphenidate released from the serdexmethylphenidate conjugate when compared to an equivalent molar amount of unconjugated d-methylphenidate following intravenous or intranasal administration of the composition to a human or animal subject. In another aspect, the lower AUC is about 10% to about 15% of the AUC of the unconjugated d-methylphenidate after intravenous administration to a human or animal subject. In another aspect, the lower Cₘₐₓ is about 20% of the Cₘₐₓ of the unconjugated d-methylphenidate after intravenous administration to a human or animal subject.

In another aspect, the composition provides a lower Take Drug Again score at 12 and 24 hours post-dose administration when compared to an equivalent molar amount of the unconjugated d-methylphenidate following intravenous administration of the composition to a human or animal subject. In another aspect, the composition provides a lower maximum (Eₘₐₓ) Feeling High score when compared to an equivalent molar amount of the unconjugated d-methylphenidate following intravenous administration of the composition to a human or animal subject. In yet another aspect, the composition provides a lower maximum (Eₘₐₓ) Good Effects score when compared to an equivalent molar amount of unconjugated d-methylphenidate following intravenous administration of the composition to a human or animal subject.

In another aspect, the composition provides a Take Drug Again score at 12 and 24 hours post-dose administration that is not substantially different when compared to a placebo following intravenous administration of the composition to a human or animal subject. In another aspect, the composition provides a maximum (Eₘₐₓ) Feeling High score that is substantially similar when compared to a placebo following intravenous administration of the composition to a human or animal subject. In yet another aspect, the composition provides a lower Overall Drug Liking score at 12 and 24 hours post-dose administration when compared to an equivalent molar amount of unconjugated d-methylphenidate following intravenous administration of the composition to a human or animal subject. In another aspect, the composition provides an Overall Drug Liking score at 12 and 24 hours post-dose administration that is substantially similar when compared to a placebo following intravenous administration of the composition to a human or animal subject. In another aspect, the composition provides a maximal (Eₘₐₓ) Feeling High score that is substantially similar when compared to a placebo following intravenous administration of the composition to a human or animal subject. In another aspect, the composition provides a maximal (Eₘₐₓ) Good Effects score that is substantially similar when compared to a placebo following intravenous administration of the composition to a human or animal subject.

In another aspect, there is a substantial difference in the median maximum (Eₘₐₓ) Drug Liking score when the composition is compared to an equivalent molar amount of unconjugated d-methylphenidate following intravenous administration to a human or animal subject.

In another aspect, the median maximum (Eₘₐₓ) Drug Liking score is substantially similar when the composition is compared to a placebo following intravenous administration to a human or animal subject.

In another aspect, there is a substantial difference in the median maximum (Eₘₐₓ) Overall Drug Liking score and the median Overall Drug Liking scores at 12 and 24 hours post-dose administration when the composition is compared to an equivalent molar amount of unconjugated d-methylphenidate following intravenous administration to a human or animal subject.

In another aspect, the median maximum (Eₘₐₓ) Overall Drug Liking score and the median Overall Drug Liking scores at 12 and 24 hours post-dose administration are substantially similar when the composition is compared to a placebo following intravenous administration to a human or animal subject.

In another aspect, there is a substantial difference in the mean Take Drug Again scores at 12 and 24 hours post-dose administration when the composition is compared to an equivalent molar amount of unconjugated d-methylphenidate following intravenous administration to a human or animal subject.

In yet another aspect, the mean Take Drug Again scores at 12 and 24 hours post-dose administration are not substantially different when the composition is compared to a placebo following intravenous administration to a human or animal subject.

In another aspect, there is a substantial difference in the median maximum (Eₘₐₓ) Feeling High score when the composition is compared to an equivalent molar amount of unconjugated d-methylphenidate following intravenous administration to a human or animal subject.

In another aspect, the mean maximum (Eₘₐₓ) Feeling High score is substantially similar when the composition is compared to a placebo following intravenous administration to a human or animal subject.

In yet another aspect, there is a substantial difference in the median maximum (Eₘₐₓ) Good Effects score when the composition is compared to an equivalent molar amount of unconjugated d-methylphenidate following intravenous administration to a human or animal subject.

In another aspect, the mean maximum (Eₘₐₓ) Good Effects score is substantially similar when the composition is compared to a placebo following intravenous administration to a human or animal subject.

Another aspect of the present technology includes a method for attenuating or reducing one or more adverse effects associated with administration of a composition comprising d-methylphenidate to a human or animal subject in need thereof, comprising replacing at least a portion of the methylphenidate to be administered with a composition comprising serdexmethylphenidate, and administering the composition comprising serdexmethylphenidate to the human or animal subject.

Another aspect of the present technology includes a method of minimizing adverse effects in a human or animal subject undergoing treatment with a composition comprising unconjugated methylphenidate said method comprising the steps of a) replacing the treatment with a composition comprising unconjugated methylphenidate with a treatement comprising a therapeutically effective amount of a composition comprising serdexmethylphenidate, or comprising a therapeutically effective amount of serdexmethylphenidate and unconjugated methylphenidate and b) administering said composition of serdexmethylphenidate, or serdexmethylphenidate and unconjugated methylphenidate to a human or animal subject in need thereof.

Another aspect of the present technology includes a method of minimizing adverse effects in a human or animal subject undergoing treatment for ADHD, where the adverse effects result from administration of a composition comprising unconjugated methylphenidate, comprising the steps of selecting a human or animal subject undergoing treatment for ADHD, replacing the treatment with a composition comprising unconjugated methylphenidate with a treatment with a therapeutically effective amount of a composition comprising serdexmethylphenidate, or comprising serdexmethylphenidate and unconjugated methylphenidate, and administering said composition of serdexmethylphenidate, or serdexmethylphenidate and unconjugated methylphenidate to a human or animal subject in need thereof. In yet another aspect, the composition comprising serdexmethylphenidate additionally comprises 0 to about 10% by weight of unconjugated d-methylphenidate, based on the total combined weight of d-methylphenidate active contained in the unconjugated d-methylphenidate and the serdexmethylphenidate conjugate.

In another aspect of the present technology, the human subject is a member selected from the group consisting of a pediatric subject, an elderly subject, a normative subject, a neonatal subject, an adolescent subject, and an adult subject. As used herein “normative subject(s)” is a human or animal (of any age) who may benefit from stimulation of the central nervous system, including but not limited to ADHA, ADD, and similar diseases or disease states or conditions. As used herein, “Neonates” are humans ages 0 to <1 month, “Infants” are humans ages 1 month to <2 years, “Children” are humans ages 2 to <12 years, “Adolescents” are humans ages 12 to <17 years, “Adults” are humans age 17 years and older, and “Elderly” are humans age 65 years and older.

In yet another aspect of the present technology, administration is selected from the group consisting of oral, intravenous, intranasal, and transdermal administration. In yet a further aspect of the present technology composition is in a dosage form selected from the group consisting of a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable table, and a suspension.

In another aspect, the one or more adverse effects is selected from the group consisting of cardiac disorders, eye disorders, gastrointestinal disorders, general disorders and administration site conditions, investigations, nervous system disorders, psychiatric disorders, skin and subcutaneous disorders, metabolism and nutrition disorders, musculoskeletal and connective tissue disorders, vascular disorders, and combinations thereof. In yet another aspect the adverse effects are selected from the group consisting of abdominal discomfort, abdominal pain, abnormal liver function ranging from transaminase elevation to severe hepatic injury, affect lability, agitation, anaphylaxis, anemia, angina pectoris, angioneurotic edema, anorexia, anxiety, arrhythmias, arthralgia, asthenia, back pain, blurred vision, bradycardia, bruxism (teeth grinding, jaw clenching), bullous conditions, cerebral hemorrhages and cerebrovascular accidents), cerebrovascular disorders (including vasculitis), change in sustained attention, chest pain, constipation, convulsions, cough, decreased appetite, depressed mood, depression, diarrhea, difficulties in visual accommodation, diplopia, disorientation, dizziness, drowsiness, dry mouth, dyskinesia including choreoathetoid movements, dyspepsia, dyspnea, emotional disorder, energy increased, eruptions, erythema, erythema multiforme rash, euphoria, exanthemas, exfoliative dermatitis, extrasystole, fatigue, feeling abnormal, feeling cold, feeling hot, feeling jittery, feeling of relaxation, fever, fixed drug eruption, flushing, gynecomastia, headache, hematuria, hyperhidrosis, hyperpyrexia, hypersensitivity reactions such as auricular swelling including angioedema, increased blood pressure, insomnia, irritability, jittery, joint pain, leukopenia, libido changes, logorrhoea (excessive talking, chattiness), mania, migraine, mood swings, muscle cramps, muscle tightness, muscle twitching, myalgia, mydriasis, myocardial infarction, nasopharyngitis, nausea, neck pain, nightmares, obsessive-compulsive disorder, palpitations, pancytopenia, paraesthesia (tingling), peripheral coldness, pharyngolaryngeal pain, phonophobia (fear of loud sounds), priapism, pruritus, psychosis (sometimes with visual and tactile hallucinations), Raynaud's phenomenon, reduced weight gain, restlessness, rhabdomyolysis, scalp hair loss, serotonin syndrome in combination with serotonergic drugs, sinus tachycardia, skin rash or hives, somnolence (sleepiness), sudden cardiac death, suppression of growth, supraventricular tachycardia, tachycardia, thrombocytopenia, thrombocytopenic purpura, tics, tremor, twitching (described as motor or vocal tics), urticaria, ventricular extrasystole, vomiting, weight loss, xerostomia, and combinations thereof.

In another aspect, the oral administration of the composition of the present invention results in reduced adverse effects when compared with a molar equivalent amount of unconjugated d-methylphenidate.

At least one aspect of the present technology includes at least one method of treating or preventing attention deficit hyperactivity disorder symptoms in a human subject comprising administering to the subject a composition comprising serdexmethylphenidate, wherein, following administration of the composition, the human or animal subject has a Cₘₐₓ, AUCₗₐₛₜ, and/or AUC_{inf} of d-methylphenidate active from the composition administered to the human or animal subject that is proportional across at least about a 1.5-fold dose range. In another aspect, following administration of the composition of the present technology each of the Cₘₐₓ, AUCₗₐₛₜ, and/or AUC_{inf} is dose-proportional across at least about a 5-fold dose range. In another aspect, following administration of the composition of the present technology each of the Cₘₐₓ, AUCₗₐₛₜ, and/or AUC_{inf} is dose-proportional across at least about a 10-fold dose range. In another aspect, following administration of the composition of the present technology each of the AUCₗₐₐₛₜ and/or AUC_{inf} is dose-proportional across at least about a 15-fold dose range. In another aspect, following administration of the composition of the present technology AUC_{inf} is dose-proportional across at least about a 25-fold dose range. In another aspect, following administration of the composition of the present technology AUC_{inf} is dose-proportional across at least about a 50-fold dose range. In another aspect, following administration of the composition of the present technology AUC_{inf} is dose-proportional across at least about a 100-fold dose range. In yet another aspect, following administration of the composition of the present technology Cₘₐₓ, AUCₗₐₛₜ, and/or AUC_{inf} of d-methylphenidate active from the composition is dose-proportional across about a 6-fold, about a 11-fold, and about a 82-fold dose range, respectively.

Another aspect of the present technology includes at least one method of treating a human or animal subject having at least one disorder or condition requiring stimulation of the central nervous system of the human or animal subject, comprising administering to the human or animal subject a pharmaceutically effective amount of a composition comprising serdexmethylphenidate, wherein the administration treats at least one disorder, or condition requiring stimulation of the central nervous system of the human or animal subject, and wherein the Cₘₐₓ, AUCₗₐₛₜ, and AUC_{inf} of d-methylphenidate active from the composition administered to the human or animal subject are proportional across at least a 1.5-fold dose range. In another aspect, following administration of the composition of the present technology each of the Cₘₐₓ, AUCₗₐₛₜ, and AUC_{inf} is dose-proportional across at least a 5-fold dose range. In another aspect, following administration of the composition of the present technology each of the Cₘₐₓ, AUCₗₐₛₜ, and AUC_{inf} is dose-proportional across at least a 10-fold dose range. In another aspect, following administration of the composition of the present technology each of the AUCₗₐₛₜ and AUC_{inf} is dose-proportional across at least a 15-fold dose range. In another aspect, following administration of the composition of the present technology AUC_{inf} is dose-proportional across at least a 25-fold dose range. In another aspect, following administration of the composition of the present technology AUC_{inf} is dose-proportional across at least a 50-fold dose range. In another aspect, following administration of the composition of the present technology AUC_{inf} is dose-proportional across at least a 100-fold dose range. In yet another aspect, following administration of the composition of the present technology Cₘₐₓ, AUCₗₐₛₜ, and AUC_{inf} of d-methylphenidate active from the composition is dose-proportional across a 6-fold, 11-fold, and 82-fold dose range, respectively.

In another aspect of the present technology, the serdexmethylphenidate in the composition is co-formulated with unconjugated d-methylphenidate.

In yet another aspect, daily administration of the composition provides a steady-state plasma concentration of released d-methylphenidate after about 24 hours of once-a-day dosing administration, alternatively after about 48 hours of once-a-day dosing administration, alternatively after about 72 hours of once-a-day dosing administration, alternatively after about 96 hours of once-a-day dosing administration, or alternatively after about 120 hours of once-a-day dosing administration.

In another aspect, the composition of the present invention has a dose mixture of about 1 mg to about 20 mg d-methylphenidate hydrochloride and about 20 mg to about 160 mg serdexmethylphenidate chloride, alternatively about 6 mg d-methylphenidate hydrochloride and about 28 mg serdexmethylphenidate chloride, alternatively about 9 mg d-methylphenidate hydrochloride and about 42 mg serdexmethylphenidate chloride, alternatively about 8 mg d-methylphenidate hydrochloride and about 64 mg serdexmethylphenidate chloride, alternatively about 12 mg d-methylphenidate hydrochloride and about 56 mg serdexmethylphenidate chloride, or alternatively about 16 mg d-methylphenidate hydrochloride and about 48 mg serdexmethylphenidate chloride.

Another aspect of the present technology includes at least one pharmaceutical kit comprising at least two sets of doses in a package, each set having a amount of individual doses in the set, wherein each individual dose in one set comprises a composition comprising unconjugated d-methylphenidate, and each individual dose in a second set comprises a composition comprising serdexmethylphenidate, and instructions for use. The at least two combined individual doses of the at least two sets of doses are therapeutically effective.

In another aspect, the instructions for use comprise a method of treating or preventing attention deficit hyperactivity disorder symptoms in a human or animal subject.

At least one aspect of the present technology includes a pharmaceutical composition for treating a disorder or condition requiring stimulation of the central nervous system comprising a serdexmethylphenidate conjugate having the following chemical formula:

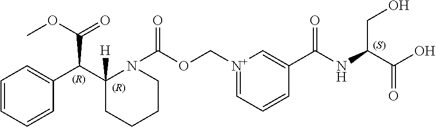

wherein administration results in minimized, reduced and/or slower onset of adverse effects as compared to compositions comprising unconjugated d-methylphenidate administered at equimolar doses.

In another aspect, the composition prevents at least one methylphenidate-related adverse effect after oral, intranasal, and/or intravenous administration to a human or animal subject when compared to an equivalent molar amount of administered unconjugated d-methylphenidate.

In another aspect of the present technology the disorder or condition requiring the stimulation of the central nervous system is selected from the group consisting of ADD (technically ADHD Predominantly Inattentive Type), ADHD with tics, ADHD with Tourette syndrome, adjunctive therapy in major depressive disorder, amphetamine use disorder, Asperger's disorder, attention-deficit hyperactivity disorder (ADHD), autism, autistic spectrum disorder, binge eating disorder, bipolar disorder, chemotherapy-associated fatigue, chronic fatigue syndrome, cocaine dependence, cocaine use disorder, depression, eating disorder, excessive daytime sleepiness (EDS), excessive sleepiness associated with obstructive sleep apnea, excessive sleepiness associated with shift work disorder, idiopathic hypersomnia, insomnia, major depressive disorder narcolepsy, methamphetamine use disorder, multiple sclerosis-associated fatigue, narcolepsy with cataplexy, obesity, pervasive developmental disorder, rejection sensitive dysphoria, schizophrenia, sleep disorder, and stimulant dependence.

Another aspect of the present invention is a method of treating a patient having Attention Deficit Hyperactivity Disorder (ADHD), comprising orally administering to the patient a pharmaceutically effective amount of a composition comprising serdexmethylphenidate and dexmethylphenidate in an amount of 26 mg serdexmethylphenidate and 5.2 mg dexmethylphenidate. In a further aspect, the serdexmethylphenidate is in the form of serdexmethylphenidate chloride, and wherein the 26 mg serdexmethylphenidate is equivalent to 28 mg serdexmethylphenidate chloride. In another aspect, the dexmethylphenidate is in the form of dexmethylphenidate hydrochloride, and wherein the 5.2 mg dexmethylphenidate is equivalent to 6 mg dexmethylphenidate hydrochloride. In yet a further aspect, the combined molar does of serdexmethylphenidate and dexmethylphenidate is equivalent to 20 mg of dexmethylphenidate hydrochloride. In yet another aspect, the 20 mg of dexmethylphenidate hydrochloride is equivalent to 17.3 mg dexmethylphenidate free base.

In one aspect of the method the composition is in the form of a capsule or tablet. In another aspect, the capsule is an immediate release capsule. In yet another aspect, the capsule or tablet is contained in a blister pack. In another aspect, the capsule or tablet is administered as a dosage of 1 capsule or tablet every day. In yet a further aspect, the dosage is of 1 capsule or tablet taken in the morning. In another aspect, the dosage is administered with or without food. In yet a further aspect, the dosage is administered with food. In another aspect, the dosage is in the form of a capsule and the capsule is opened and the composition of the capsule is sprinkled onto a tablespoon of a semi-solid or a liquid. In yet another aspect, the semi-solid is applesauce. In yet another aspect, the liquid is water.

Another aspect of the present invention is a method of treating a patient having Attention Deficit Hyperactivity Disorder (ADHD), comprising orally administering to the patient a pharmaceutically effective amount of a composition comprising serdexmethylphenidate and dexmethylphenidate in an amount of 39 mg serdexmethylphenidate and 7.8 mg dexmethylphenidate. In a further aspect, the serdexmethylphenidate is in the form of serdexmethylphenidate chloride, and wherein the 39 mg serdexmethylphenidate is equivalent to 42 mg serdexmethylphenidate chloride. In yet another aspect, the dexmethylphenidate is in the form of dexmethylphenidate hydrochloride, and wherein the 7.8 mg dexmethylphenidate is equivalent to 9 mg dexmethylphenidate hydrochloride. In a further aspect, the combined molar does of serdexmethylphenidate and dexmethylphenidate is equivalent to 30 mg of dexmethylphenidate hydrochloride. In yet another aspect, the 30 mg of dexmethylphenidate hydrochloride is equivalent to 25.9 mg dexmethylphenidate free base.

In one aspect of the method the composition is in the form of a capsule or tablet. In another aspect, the capsule is an immediate release capsule. In yet another aspect, the capsule or tablet is contained in a blister pack. In another aspect, the capsule or tablet is administered as a dosage of 1 capsule or tablet every day. In yet a further aspect, the dosage is of 1 capsule or tablet taken in the morning. In another aspect, the dosage is administered with or without food. In yet a further aspect, the dosage is administered with food. In another aspect, the dosage is in the form of a capsule and the capsule is opened and the composition of the capsule is sprinkled onto a tablespoon of a semi-solid or a liquid. In yet another aspect, the semi-solid is applesauce. In yet another aspect, the liquid is water.

Another aspect of the present invention is a method of treating a patient having Attention Deficit Hyperactivity Disorder (ADHD), comprising orally administering to the patient a pharmaceutically effective amount of a composition comprising serdexmethylphenidate and dexmethylphenidate in an amount of 52 mg serdexmethylphenidate and 10.4 mg dexmethylphenidate. In another aspect, the serdexmethylphenidate is in the form of serdexmethylphenidate chloride, and wherein the 52 mg serdexmethylphenidate is equivalent to 56 mg serdexmethylphenidate chloride. In yet another aspect, the dexmethylphenidate is in the form of dexmethylphenidate hydrochloride, and wherein the 10.4 mg dexmethylphenidate is equivalent to 12 mg dexmethylphenidate hydrochloride. In a further aspect, the combined molar does of serdexmethylphenidate and dexmethylphenidate is equivalent to 40 mg of dexmethylphenidate hydrochloride. In yet a further aspect, the 40 mg of dexmethylphenidate hydrochloride is equivalent to 34.6 mg dexmethylphenidate free base.

In one aspect of the method the composition is in the form of a capsule or tablet. In another aspect, the capsule is an immediate release capsule. In yet another aspect, the capsule or tablet is contained in a blister pack. In another aspect, the capsule or tablet is administered as a dosage of 1 capsule or tablet every day. In yet a further aspect, the dosage is of 1 capsule or tablet taken in the morning. In another aspect, the dosage is administered with or without food. In yet a further aspect, the dosage is administered with food. In another aspect, the dosage is in the form of a capsule and the capsule is opened and the composition of the capsule is sprinkled onto a tablespoon of a semi-solid or a liquid. In yet another aspect, the semi-solid is applesauce. In yet another aspect, the liquid is water.

In one aspect of the method the serdexmethylphenidate has the following structure

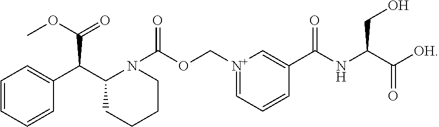 [SMILES: COC(=O)[C@H](c1ccccc1)[C@H]1CCCCN1C(=O)OC[n+]1cccc(C(=O)N[C@@H](CO)C(=O)O)c1]

In another aspect of the method, the serdexmethylphenidate chloride has the following structure

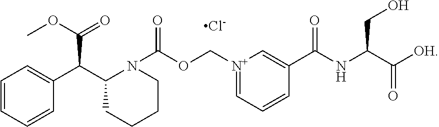 [SMILES: COC(=O)[C@H](c1ccccc1)[C@H]1CCCCN1C(=O)OC[n+]1cccc(C(=O)N[C@@H](CO)C(=O)O)c1 ; [Cl-]]

At least one aspect of the present technology includes at least one process for the preparation of serdexmethylphenidate conjugate polymorphs comprising the step of using crystallization conditions to isolate a free-base and salt forms and/or by ball-milling such forms.

Moreover, the present technology may provide at least one method of treating one or more subjects (human or animal) having at least one disease, disorder, syndrome, or condition mediated by controlling, preventing, limiting, or inhibiting neurotransmitter uptake/re-uptake or hormone uptake/re-uptake comprising administering a pharmaceutically and/or therapeutically effective amount of the serdexmethylphenidate conjugate of the present technology to one or more of such subjects.

In yet another embodiment, the present technology provides at least one method of minimizing one or more adverse effects in one or more human or animal subjects, wherein the adverse effects result from administration of a composition comprising unconjugated methylphenidate, the method comprising the step of replacing administration of a composition comprising unconjugated methylphenidate with administration of a therapeutically effective amount of a composition comprising serdexmethylphenidate of the present technology, or comprising serdexmethylphenidate and unconjugated methylphenidate.

In at least some embodiments, compositions comprising serdexmethylphenidate of the present technology exhibit reduced plasma or blood concentrations of released d-methylphenidate when administered intranasally or intravenously to a human or animal subject, as compared to the plasma concentrations of released d-methylphenidate following administration of unconjugated d-methylphenidate at equimolar amounts to a human or animal subject.

In at least one embodiment, the present technology provides at least one composition comprising (a) unconjugated methylphenidate, wherein the unconjugated d-methylphenidate comprises d-methylphenidate, and (b) serdexmethylphenidate having the following chemical formula:

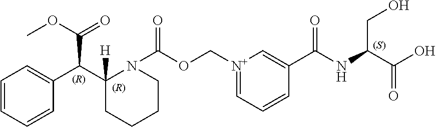

or a pharmaceutically acceptable salt thereof, wherein after administration of the composition, the composition has an onset of action at about 0.5 to about 2.0 hours post-dose, alternatively at about 0.5 to about 1.0 hours post-dose, alternatively at about 0.75 to about 1.5 hours post-dose as compared to a placebo; and a duration of efficacy until about 10 to about 16 hours post-dose, alternatively until about 10 to about 13 hours post-dose, alternatively until about 10 to about 12 hours post-dose, alternatively until about 14 to about 16 hours post-dose; and total duration of efficacy of about 0.5 to about 16 hours post-dose, alternatively about 0.5 to about 13 hours post-dose, alternatively about 1 to about 10 hours post-dose as compared to placebo.

In some embodiments, the serdexmethylphenidate conjugate may have the following structure:

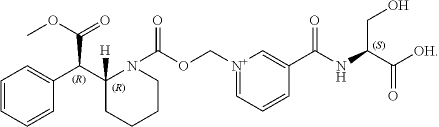

In some embodiments, novel intermediates are produced during the process of synthesizing serdexmethylphenidate. In yet another embodiment, novel metabolites and/or novel degradants are produced during the breakdown (for example, metabolic processes) of serdexmethylphenidate either in vivo and/or in vitro.

### BRIEF DESCRIPTION OF THE DRAWINGS

FIG. 1. Example synthetic scheme for the synthesis of the serdexmethylphenidate conjugate of the present technology.

FIG. 2. Oral PK curve of the plasma concentration-time profiles for three dose mixtures of d-methylphenidate/serdexmethylphenidate after single-dose administration in human subjects.

FIG. 3. Oral PK curve of the plasma concentration-time profile following 4 oral doses of d-methylphenidate/serdexmethylphenidate, 12/56 mg, administered in adult human subjects once every 24 hours.

FIGS. 4A-C. After single-dose KP415 administration analyses using a prespecified power analysis indicated that KP415 was dose-proportional across a 6.5-(FIG. 4A), 11.1-(FIG. 4B), and 82.7-(FIG. 4C) fold range of doses for Cₘₐₓ, AUCₗₐₛₜ, and AUC_{inf}, respectively.

FIG. 5. d-methylphenidate Time-Plasma Concentration Profile.

FIG. 6. Plasma concentration-time profile of d-methylphenidate released from single doses of d-methylphenidate hydrochloride/serdexmethylphenidate chloride 6/28, 9/42, and 12/56 mg after oral administration in human subjects.

FIG. 7. Plot of the ratio of the dose-normalized geometric mean values (Rdnm) of Cₘₐₓ plus associated 90% confidence interval (CI) vs. dose ratio (r) as predicted by a power model. The model predicts definitive dose proportionality for Cₘₐₓ of d-methylphenidate (d-MPH) in the region from r=1 through r=rho1 and Rdnm=θ_{L} through Rdnm=θ_{H} (where θ_{L}=0.8 and θ_{H}=1.25 represent the lower and upper bounds of the acceptance interval).

FIG. 8. Plot of the ratio of the dose-normalized geometric mean values (Rdnm) of AUCₗₐₛₜ plus associated 90% confidence interval (CI) vs. dose ratio (r) as predicted by a power model. The model predicts definitive dose proportionality for AUCₗₐₛₜ of d-methylphenidate (d-MPH) in the region from r=1 through r=rho1 and Rdnm=θ_{L} through Rdnm=θ_{H} (where θ_{L}=0.8 and θ_{H}=1.25 represent the lower and upper bounds of the acceptance interval).

FIG. 9. Plot of the ratio of the dose-normalized geometric mean values (Rdnm) of AUC_{inf} plus associated 90% confidence interval (CI) vs. dose ratio (r) as predicted by a power model. The model predicts definitive dose proportionality for AUC_{inf} of d-methylphenidate (d-MPH) in the region from r=1 through r=rho1 and Rdnm=θ_{L} through Rdnm=θ_{H} (where θ_{L}=0.8 and θ_{H}=1.25 represent the lower and upper bounds of the acceptance interval).

FIG. 10. Plasma concentration-time profile of d-methylphenidate (d-MPH) released from d-methylphenidate hydrochloride (d-MPH HCl)/serdexmethylphenidate chloride (SDX Cl), 12/56 mg after oral administration of Dose 1 (Day 1) and Dose 4 (Day 4) in human subjects.

FIG. 11. Plasma concentration-time profile of serdexmethylphenidate (SDX) released from d-methylphenidate hydrochloride (d-MPH)/serdexmethylphenidate chloride (SDX Cl), 12/56 mg after oral administration of Dose 1 (Day 1) and Dose 4 (Day 4) in human subjects.

FIG. 12. IV Study (KP415.A03) Plasma concentration-time profile of d-methylphenidate released from single doses of 30 mg serdexmethylphenidate chloride and 15 mg d-methylphenidate hydrochloride after intravenous administration in human subjects.

FIG. 13. At the moment Drug Liking VAS scores following intravenous administration of single doses of 30 mg serdexmethylphenidate chloride, 15 mg d-methylphenidate hydrochloride, and placebo in human subjects.

FIG. 14. At the moment Feeling High VAS scores following intravenous administration of single doses of 30 mg serdexmethylphenidate chloride, 15 mg d-methylphenidate hydrochloride, and placebo in human subjects.

FIG. 15. At the moment Good Effects VAS scores following intravenous administration of single doses of 30 mg serdexmethylphenidate chloride, 15 mg d-methylphenidate hydrochloride, and placebo in human subjects.

FIG. 16. Mean scores for pharmacodynamic endpoints Drug Liking Eₘₐₓ, Overall Drug Liking Eₘₐₓ, and Overall Drug Liking at 12 and 24 hours measured on a bipolar VAS following intravenous administration of single doses of 30 mg serdexmethylphenidate chloride, 15 mg d-methylphenidate hydrochloride, and placebo in human subjects.

FIG. 17. Median scores for pharmacodynamic endpoints Drug Liking Eₘₐₓ, Overall Drug Liking Eₘₐₓ, and Overall Drug Liking at 12 and 24 hours measured on a bipolar VAS following intravenous administration of single doses of 30 mg serdexmethylphenidate chloride, 15 mg d-methylphenidate hydrochloride, and placebo in human subjects.

FIG. 18. Mean scores for pharmacodynamic endpoints Take Drug Again Eₘₐₓ, Take Drug Again at 12 and 24 hours, Feeling High Eₘₐₓ, and Good Effects Eₘₐₓ measured on a unipolar VAS following intravenous administration of single doses of 30 mg serdexmethylphenidate chloride, 15 mg d-methylphenidate hydrochloride, and placebo in human subjects.

FIG. 19. Median scores for pharmacodynamic endpoints Take Drug Again Eₘₐₓ, Take Drug Again at 12 and 24 hours, Feeling High Eₘₐₓ, and Good Effects Eₘₐₓ measured on a unipolar VAS following intravenous administration of single doses of 30 mg serdexmethylphenidate chloride, 15 mg d-methylphenidate hydrochloride, and placebo in human subjects.

FIG. 20. Median of differences in Drug Liking Eₘₐₓ measured on a bipolar VAS for the comparisons of 15 mg d-methylphenidate hydrochloride vs. 30 mg serdexmethylphenidate chloride and 30 mg serdexmethylphenidate chloride vs. placebo.

FIG. 21. Median of differences in Overall Drug Liking Eₘₐₓ, and Overall Drug Liking at 12 and 24 hours post-dose measured on a bipolar VAS for the comparison of 15 mg d-methylphenidate hydrochloride vs 30 mg serdexmethylphenidate chloride and 30 mg; and mean differences in Overall Drug Liking Eₘₐₓ, and Overall Drug Liking at 12 and 24 hours post-dose for the comparison of 30 mg serdexmethylphenidate chloride and 30 mg vs placebo.

FIG. 22. Mean differences in Take Drug Again Eₘₐₓ, and Take Drug Again at 12 and 24 hours post-dose measured on a unipolar VAS for the comparisons of 15 mg d-methylphenidate hydrochloride vs. 30 mg serdexmethylphenidate chloride and 30 mg serdexmethylphenidate chloride vs. placebo.

FIG. 23. Median of differences in Feeling High Eₘₐₓ and Good Effects Eₘₐₓ measured on a unipolar VAS for the comparison of 15 mg d-methylphenidate hydrochloride vs. 30 mg serdexmethylphenidate chloride and 30 mg; and mean differences in Feeling High Eₘₐₓ and Good Effects Eₘₐₓ for the comparison of 30 mg serdexmethylphenidate chloride and 30 mg vs. placebo.

FIG. 24. ADHD efficacy study design schematic.

FIG. 25. Comparison of SKAMP-C change from baseline for d-methylphenidate/serdexmethylphenidate vs. placebo using Visit 5 baseline scores.

FIG. 26. Comparison of SKAMP-C change from baseline for d-methylphenidate/serdexmethylphenidate vs. placebo using Visit 6 baseline scores.

FIG. 27. Comparison of absolute SKAMP-C scores for d-methylphenidate/serdexmethylphenidate vs. placebo.

FIG. 28. Comparison of mean absolute SKAMP-C scores for d-methylphenidate/serdexmethylphenidate vs. placebo.

FIG. 29. Comparison of SKAMP-A change from baseline for d-methylphenidate/serdexmethylphenidate vs. placebo using Visit 5 baseline scores.

FIG. 30. Comparison of SKAMP-A change from baseline for d-methylphenidate/serdexmethylphenidate vs. placebo using Visit 6 baseline scores.

FIG. 31. Comparison of absolute SKAMP-A scores for d-methylphenidate/serdexmethylphenidate vs. placebo.

FIG. 32. Comparison of mean absolute SKAMP-A scores for d-methylphenidate/serdexmethylphenidate vs. placebo.

FIG. 33. Comparison of SKAMP-D change from baseline for d-methylphenidate/serdexmethylphenidate vs. placebo using Visit 5 baseline scores.

FIG. 34. Comparison of SKAMP-D change from baseline for d-methylphenidate/serdexmethylphenidate vs. placebo using Visit 6 baseline scores.

FIG. 35. Comparison of absolute SKAMP-D scores for d-methylphenidate/serdexmethylphenidate vs. placebo.

FIG. 36. Comparison of mean absolute SKAMP-D scores for d-methylphenidate/serdexmethylphenidate vs. placebo.

FIG. 37. ADHD-RS-5 Inattention scores.

FIG. 38. ADHD-RS-5 Hyperactivity scores.

FIG. 39. ADHD-RS-5 total scores.

FIG. 40. Comparison of ADHD-RS-5 scores from Visit 5 vs. Visit 2.

FIG. 41. Comparison of PERMP-A change from Visit 5 baseline for d-methylphenidate/serdexmethylphenidate vs. placebo.

FIG. 42. Comparison of PERMP-C change from Visit 5 baseline for d-methylphenidate/serdexmethylphenidate vs. placebo.

FIG. 43. Comparison of PERMP % correct change from Visit 5 baseline for d-methylphenidate/serdexmethylphenidate vs. placebo.

FIG. 44. Comparison of PERMP total score change from Visit 5 baseline for d-methylphenidate/serdexmethylphenidate vs. placebo.

FIG. 45. Comparison of absolute PERMP-A score for d-methylphenidate/serdexmethylphenidate vs. placebo.

FIG. 46. Comparison of absolute PERMP-C score for d-methylphenidate/serdexmethylphenidate vs. placebo.

FIG. 47. Comparison of absolute PERMP % correct score for d-methylphenidate/serdexmethylphenidate vs. placebo.

FIG. 48. Comparison of absolute PERMP score for d-methylphenidate/serdexmethylphenidate vs. placebo.

FIGS. 49a-d. Comparison of WREMB-R assessment scores for d-methylphenidate/serdexmethylphenidate vs. placebo showing morning change from Visit 2 baseline (49a), evening change from Visit 2 baseline (49b), morning scores (49c), and evening scores (49d).

FIG. 50. Conners 3-P T-scores for d-methylphenidate/serdexmethylphenidate.

FIG. 51. Conners 3-P T-scores for placebo.

FIG. 52. Comparison of change in Conners 3-P T-scores from Visit 2 baseline for d-methylphenidate/serdexmethylphenidate vs. placebo.

FIG. 53 is a plot of change in SKAMP-C scores from predose Visit 6 vs time.

FIG. 54 is a plot of absolute SKAMP-C scores vs time.

FIG. 55 is a graph showing maximum (Eₘₐₓ) Drug Liking scores for intravenous administration.

FIG. 56 is a graph showing the IN Study (KP415.A02) Plasma concentration-time profile of d-methylphenidate released from single doses of 80 mg serdexmethylphenidate chloride and 40 mg d-methylphenidate hydrochloride after intranasal administration in human subjects.

FIG. 57 is a comparison showing at the moment Drug Liking VAS scores following intranasal administration of single doses of 80 mg serdexmethylphenidate chloride, 40 mg d-methylphenidate hydrochloride, and placebo in human subjects.

FIG. 58 is a comparison of Feeling High scores for intranasal administration.

FIG. 59 is a comparison of Good Effects scores for intranasal administration.

FIG. 60 is a comparison of Bad Effects scores for intranasal administration.

FIG. 61 is a comparison of Alertness scores for intranasal administration.

FIG. 62 is a comparison of Any Effects scores for intranasal administration.

FIG. 63 is a proposed metabolic pathway of serdexmethylphenidate.

FIG. 64 is a comparison of LS Mean SKAMP-Combined Score Change from Pre-dose after Treatment with serdexmethylphenidate/d-methylphenidate or Placebo.

### DETAILED DESCRIPTION OF THE INVENTION

The present technology provides one or more compositions comprising at least one serdexmethylphenidate conjugate that provides one or more beneficial properties, including, but not limited to minimizing the adverse effects in one or more human or animal subjects, wherein at least some of the adverse effects result from administration of at least one composition comprising unconjugated d-methylphenidate, as further described herein.

The use of the term “methylphenidate” herein is meant to include any of the stereoisomer forms of methylphenidate, including the four stereoisomers: d-erythro-methylphenidate, l-erythro-methylphenidate, d-threo-methylphenidate and l-threo-methylphenidate and the salts and derivatives thereof. Methylphenidate is interchangeable with methyl phenyl(piperidin-2-yl)acetate. The term “methylphenidate” includes all salt forms. Methylphenidate is also known by its trade name Concerta® (commercially available from Janssen Pharmaceuticals, Inc., Beerse, Belgium), Ritalin®, Ritalin® SR, Methylin®, Methylin® ER (all commercially available from Novartis International AG, of Basil, Switzerland). The methylphenidate moiety in serdexmethylphenidate used in the present technology can be any stereoisomer of methylphenidate, including, but not limited to, d-erythro-methylphenidate, l-erythro-methylphenidate, d-threo-methylphenidate and l-threo-methylphenidate. In a preferred embodiment, the conjugates contain a single d-threo-methylphenidate isomer.

The use of the term “unconjugated methylphenidate” means methyl 2-phenyl-2-(piperidin-2-yl)acetate and salts thereof.

The use of the term “d-methylphenidate” means methyl (R)-2-phenyl-2-((R)-piperidin-2-yl)acetate.

“Bioavailability”, used herein, means the proportion of a drug or other substance that enters the circulation over time when introduced into the human or animal body and so is able to have an active effect.

“Mean peak plasma concentration” or “(Cₘₐₓ)”, used herein, is defined as mean maximum plasma concentration or maximum mean plasma concentration. Cₘₐₓ is a pharmacokinetics term and refers to the maximum (or peak) plasma concentration that a drug achieves in a specified compartment or test area of the human or animal body after the drug has been administered and before the administration of a second dose.

“Maximum plasma concentration”, used herein, is the term used in pharmacokinetics to describe the maximum plasma concentration of a drug or metabolite observed after administration of a drug in a human or animal subject.

“Mean plasma concentration”, used herein, is the term used in pharmacokinetics to describe the arithmetic mean of blood plasma concentrations of multiple subjects.

“Tₘₐₓ”, used herein, is a pharmacokinetics term that describes the time at which the Cₘₐₓ is observed. After an intravenous administration, Cₘₐₓ and Tₘₐₓ are closely dependent on the experimental protocol, since the concentrations typically are decreasing after the dose.

“Maximum Effect”, “Maximum Effect Score” or “(Eₘₐₓ)”, used herein, is the term used in pharmacodynamics to describe the maximum subjective pharmacodynamic effect of a drug or metabolite observed after administration of a drug in a human or animal subject. A drug or metabolite can have multiple different pharmacodynamic effects, each having their own maximum effect or maximum effect score at similar or different times post-dose administration in a human or animal subject.

“Statistically similar,” used herein, is defined as meaning statistically not different in a population with appropriate sample size as demonstrated by an appropriate 2-sided statistical test, and/or statistically not inferior in a population with appropriate sample size within a predefined margin as demonstrated by an appropriate 1-sided statistical test. In one embodiment, for example, the margin employed for statistical testing of data collected in studies was 10 points for the comparison of unconjugated d-methylphenidate with serdexmethylphenidate, Focalin® XR with serdexmethylphenidate, phentermine with serdexmethylphenidate, and phentermine with placebo. In another embodiment, for example, the margin was 11 points for the comparison of serdexmethylphenidate with placebo. In yet a further embodiment, for example, the margin was 15 points for the comparison of Focalin® XR with placebo.

“Substantially similar”, used herein, is defined as meaning statistically similar.

“Statistically different”, used herein, is defined as meaning statistically different in a human or animal population with appropriate sample size as demonstrated by an appropriate 2-sided statistical test, and/or statistically superior in a human or animal population with appropriate sample size beyond a predefined margin as demonstrated by an appropriate 1-sided statistical test. If a margin was employed for statistical testing of data collected in studies described herein, that margin was 10 points for the comparison of unconjugated d-methylphenidate with serdexmethylphenidate, Focalin® XR with serdexmethylphenidate, phentermine with serdexmethylphenidate, and phentermine with placebo. In another embodiment, for example, the margin was 11 points for the comparison of serdexmethylphenidate with placebo. In yet a further embodiment, for example, the margin was 15 points for the comparison of Focalin® XR with placebo.

“Not substantially different”, used herein, is defined as meaning statistically different but the difference is not or is minimally clinically, pharmacologically, or pharmacodynamically meaningful as conventionally defined within the pharmaceutical, nutraceutical, or animal science industries.

“Substantially different”, used herein, is defined as meaning statistically different and the difference is clinically, pharmacologically, or pharmacodynamically meaningful as conventionally defined within the pharmaceutical, nutraceutical, or animal science industries.

“Substantially higher”, used herein, is defined as meaning statistically different and the difference represents an increase that is clinically, pharmacologically, or pharmacodynamically meaningful as conventionally defined within the pharmaceutical, nutraceutical, or animal science industries; i.e. statistically higher.

“Substantially lower”, or “statistically substantially lower”, or “statistically significantly lower” used herein, is defined as meaning statistically different and the difference represents a decrease or reduction that is clinically, pharmacologically, or pharmacodynamically meaningful as conventionally defined within the pharmaceutical, nutraceutical, or animal science industries; i.e. statistically lower.

The use of the term “dose” means the total amount of a drug or active component taken each time by an individual human or animal subject.

As used herein, the term “subject” means a human or animal, including but not limited to a human or animal patient.

The term “patient” means a human or animal subject in need of treatment.

“Overall systemic exposure”, used herein, is the term used to describe area under the curve of a plasma concentration-time plot for a drug or metabolite from time zero (dose administration or pre-dose) through the time of the last observed plasma concentration (AUCₗₐₛₜ) or extrapolated to infinity (AUC_{inf}).

AUCₗₐₛₜ is a term used in pharmacokinetics to describe the area under the curve in a plot of drug concentration in blood, serum, or plasma vs. time from time=0 (or pre-dose) to the time of the last measurable drug concentration.

AUC_{inf} is a term used in pharmacokinetics to describe the area under the curve in a plot of drug concentration in blood, serum, or plasma vs. time from time=0 (or pre-dose) to infinity.

“CL/F” or “clearance” as used here is the measurement of the volume of plasma from which a substance is completely removed per unit time. CL/F is calculated with the following formula: CL/F=Dose/AUC_{inf}.

“V_{z}/F” or “volume of distribution” as used herein means the theoretical volume that would be necessary to contain the amount of drug in the body during the terminal phase at the same concentration as in the blood plasma during the terminal phase. V_{z}/F is calculated with the following formula: V_{z}/F−(CL/F)/λ_{z}, where “λ_{z}” or “lambdaZ” is the terminal elimination rate constant.

“allometric scaling” as used herein is the ability to calculate pharmacokinetic parameters or plasma concentrations based on body weight, or body weight and dose.

Visual analog scale (VAS), used herein, is the term to describe a psychometric response scale which can be used in questionnaires. It is a measurement instrument for subjective characteristics or attitudes that cannot be directly measured.

“Drug liking” score, used herein, is the score used to assess the degree that a human participant likes a drug effect at the time the question is being asked (that is, at the moment). It is scored using a 0 to 100 point bipolar visual analogue scale (VAS) anchored in the center with a neutral anchor of “neither like nor dislike” (score of 50), on the left with “strong disliking” (score of 0) and on the right with “strong liking” (score of 100).

“Euphoria” or “Feeling High” score, used herein, is the term to describe the score used to assess the degree that a human participant is high at the time the question is being asked (that is, at the moment). It is scored using a 0 to 100 point unipolar visual analogue scale (VAS) anchored on the left with “Not at All” (score of 0) and on the right with “Extremely” (score of 100).

“Take Drug Again” score, used herein, is the term to describe the score used to assess the degree that a human participant wants to take the drug again, if given the opportunity, based on his/her opinion now, i.e., at the time the question is being asked. It is scored using a 0 to 100 point unipolar visual analogue scale (VAS) anchored on the left with “Definitely Would Not” (score of 0) and on the right with “Definitely Would” (score of 100). Alternatively, “Take Drug Again” may be scored using a 0 to 100 points bipolar VAS anchored in the center with a neutral anchor of “Do Not Care” (score of 50), on the left with “Definitely Not” (score of 0) and on the right with “Definitely Would” (score of 100).

“Overall Drug Liking” score, used herein, is the term to describe the score used to assess the human subject's global perception of drug liking (i.e., the subjective effects over the whole course of the drug experience including any carryover effects). Subjects respond to the statement “Overall, my liking for this drug is.” The question is scored using a 0-100 point bipolar VAS anchored on the left with “Strong Disliking” (score of 0); “Neither Like nor Dislike” (score of 50) in the middle, and anchored on the right with “Strong Liking” (score of 100). This scale has the advantage of the human subject being relatively less affected or unaffected by acute study drug effects (if any) by the time of the assessment.

“Good Effects” score, used herein, is the term to describe the score used to assess the degree that a human participant is feeling good drug effects at the time the question is being asked (that is, at the moment). Subjects respond to the statement “At this moment, I can feel good drug effects.” It is scored using a 0 to 100 point unipolar visual analogue scale (VAS) anchored on the left with “Not at All” (score of 0) and on the right with “Extremely” (score of 100).

“Bad Effects” score, as used herein, is the term to describe the score used to assess the degree that a participant feels bad effects at the time the question is being asked (that is, at the moment). Subjects respond to the statement “At this moment, I can feel bad drug effects.” It is scored using a 0 to 100 points unipolar VAS anchored on the left with “Definitely Not” (score of 0) and on the right with “Definitely Yes” (score of 100).

“Any Effects” score, as used herein, is the term to describe the score used to assess the degree that a participant feels any effects at the time the question is being asked (that is, at the moment). Subjects respond to the statement “At this moment, I can feel any drug effects.” It is scored using a 0 to 100 points unipolar VAS anchored on the left with “Definitely Not” (score of 0) and on the right with “Definitely Yes” (score of 100).

“Drowsiness/Alertness” score, as used herein, is the term to describe the score used to assess the degree that a participant feels alert or drowsy at the time the question is being asked (that is, at the moment). Subjects respond to the statement “At this moment, my mental state is”. It is scored using a 0 to 100 points bipolar VAS anchored in the center with a neutral anchor of “neither drowsy nor alert” (score of 50), on the left with “very drowsy” (score of 0) and on the right with “very alert” (score of 100).

“Ease of Insufflation” VAS is the measure that assesses the difficulty of snorting the study drugs. Subjects will respond to the statement “Snorting this drug was:” The question will be scored using a 0-100 points unipolar VAS anchored on the left with “Very Easy” (score of 0) and anchored on the right with “Very Difficult” (score of 100).

“Abuse related effects”, used herein, is the term to describe pharmacodynamic effects felt or experienced by a human subject following drug administration including, but not limited to, Drug Liking, Euphoria, Feeling High, Good Effects, and Alertness.

“Bipolar scale”, used herein, is the term to describe scale where measures can lie below or above a midpoint that itself represents a point of ambivalence or neutrality.

“Unipolar scale”, used herein, is the term to measure an amount between a predefined minimum and maximum.

“Maximum drug Liking” score, used herein, is the term to describe the maximum score of a series of “Drug Liking” scores collected over a period of time following drug administration.

“SKAMP” score, used herein, refers to the Swanson, Kotkin, Agler, M-Flynn, and Pelham Rating Scale used to assess the classroom behavior in children with ADHD. It is comprised of 13 items (grouped under the subcategories of attention, deportment, quality of work, and compliance), on which subjects are rated according to a 7-point scale (0=normal to 6=maximal impairment) by trained study personnel. The SKAMP-Combined (SKAMP-C) score is obtained by summing the rating values for each of the 13 items. The SKAMP-Deportment (SKAMP-D) score is a measure of behavior and comprises of 4 items. The SKAMP-Attention (SKAMP-A) score is a measure of attention and comprises 4 items. Higher SKAMP scores signify greater impairment.

“PERMP” score, used herein, refers to the Permanent Product Measure of Performance Rating Scale Skill. The test is an adjusted math test designed to assess attention in children with ADHD. The test measures attention through a subject's ability to initiate, self-monitor, and complete the math test. A Placement PERMP is performed early in the trial to assure that subjects can complete at least the basic level of math problems and to determine the appropriate level of math to be assigned during the remainder of the study. The PERMP is an individually calibrated five-page mathematics worksheet consisting of 400 problems. Subjects were instructed by site staff to work at their seats and complete as many problems as possible in 10 minutes. Performance is evaluated using two scores: The number of problems attempted (PERMP-A) and the number of problems correct (PERMP-C). Higher PERMP scores indicated better performance.

As used herein, “Weekly Rating of Evening and Morning Behavior-Revised (WREMB-R)” scale refers to an 11-item parent-rated questionnaire that was developed to assess behaviors for their severity during the morning hours (3 items) and evening hours (8 items) (Carlson 2007). The possible score for each item ranges from 0 (no difficulty) to 3 (a lot of difficulty).

As used herein, “Conners 3-P” refers to a questionnaire that provides evaluation of inattention, hyperactivity/impulsivity, learning problems, executive functioning, aggression, and peer relationships.

As used herein, “ADHD-Rating Scale-5” or “ADHD-RS-5” refers to an 18-item scale based on Diagnostic and Statistical Manual of Mental Disorders, 5th edition (DSM-5) (American Psychiatric Association 2013) criteria of ADHD that rates symptoms on a 4-point scale. Each item is scored using a combination of severity and frequency ratings from a range of 0 (reflecting no symptoms or a frequency of never or rarely) to 3 (reflecting severe symptoms or a frequency of very often), so that the total ADHD-RS-5 scores range from 0 to 54. The 18 items can be divided into two 9-item subscales: One for hyperactivity/impulsivity and the other for inattentiveness.

“Molar equivalent” as used herein, means an equal number of moles of the substance as the number of moles in a certain mass (weight) or volume of the comparison substance, e.g. a dose of d-methylphenidate that is molar equivalent to a dose of about 0.1 mg d-methylphenidate hydrochloride per day would provide the same number of moles of d-methylphenidate as from 0.1 mg of d-methylphenidate hydrochloride.

As used herein, the phrases such as “decreased,” “reduced,” “diminished” or “lowered” are meant to include at least about a 10% change in pharmacological activity, area under the curve (AUC) and/or peak plasma concentration (Cₘₐₓ) with greater percentage changes being preferred for reduction in abuse potential and overdose potential of the conjugates of the present technology as compared to unconjugated d-methylphenidate. For instance, the change may also be greater than about 10%, about 15%, about 20%, about 25%, about 35%, about 45%, about 55%, about 65%, about 75%, about 85%, about 95%, about 96%, about 97%, about 98%, about 99%, or increments therein.

“Pharmaceutically effective amount” as used herein means an amount that has a pharmacological effect. A “pharmaceutically acceptable salt” as used herein is a salt which, when used in a pharmaceutically effective amount, has at least one pharmacological effect.

“Therapeutically effective amount” as used herein means an amount effective for treating a disease or condition. A “therapeutically acceptable salt” as used herein is a pharmaceutically acceptable salt, which, when used in a therapeutically effective amount, is effective for treating a disease, condition, or syndrome.

As used herein, the term “attention deficit hyperactivity disorder” (ADHD) encompasses various sub-types of ADHD including, for example, subjects who do not show or only show weak symptoms of hyperactivity or impulsiveness, and are predominately inattentive (formerly attention deficit disorder (ADD)). Alternatively, subjects may show predominantly symptoms of hyperactivity or impulsiveness, and no or only weak symptoms of inattentiveness. Alternatively, subjects may show both symptoms of hyperactivity or impulsiveness, and symptoms of inattentiveness.

As used herein, the term “prodrug” refers to a substance that is inactive or has reduced pharmacological activity but is converted to an active drug by a chemical or biological reaction in the body. In the present technology, the serdexmethylphenidate conjugate may be a prodrug or formulated as a prodrug formulation.

As used herein, the term “unformulated” refers to compositions of therapeutic compound(s) free of excipients that significantly affect the intrinsic absorption properties of such compound(s).

The serdexmethylphenidate conjugate can be prepared so as to have a variety of different chemical forms including chemical derivatives or salts. Such serdexmethylphenidate conjugates can also be prepared to have different physical forms. For example, the serdexmethylphenidate conjugate may be amorphous, may have different crystalline polymorphs, or may exist in different solvation or hydration states, such as semi-hydrates, monohydrates, hydrates (nH₂O, when n is 0.5, 1, 2, etc.). Such polymorphs can be produced by, e.g., using crystallization conditions to isolate a free-base and salt forms and/or by ball-milling such forms.

By varying the form of the serdexmethylphenidate conjugate, it should be appreciated by those skilled in the art that it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility. Accordingly, variation of the crystalline state of the serdexmethylphenidate conjugate is one of many ways in which to modulate the physical properties thereof.

The serdexmethylphenidate conjugate can be either a positively charged (cationic) molecule, or a pharmaceutically acceptable anionic or cationic salt form or salt mixtures with any ratio between positive and negative components. In some of the preferred embodiments, the anionic salt form is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, and nitrite.

The cationic salt forms can include, but are not limited to, for example, sodium, potassium, calcium, magnesium, lithium, cholinate, lysinium, or ammonium forms, among others.

### General Structures and Definitions

Abbreviations for the components of conjugates of the present technology include: MPH stands for methylphenidate; MPH-HCl stands for methylphenidate hydrochloride; d-MPH stands for d-threo-methylphenidate; d-MPH-HCl stands for d-threo-methylphenidate hydrochloride; SDX stands for serdexmethylphenidate; Ser stands for serine; tBu stands for tert-butyl; Et stands for ethyl.

In some embodiments, the general structure of the serdexmethylphenidate conjugates that, when administered at a therapeutically effective dose, may provide reduced and/or slower onset of side effects as compared to compositions comprising unconjugated d-methylphenidate administered at equimolar doses can be represented by Formula I:

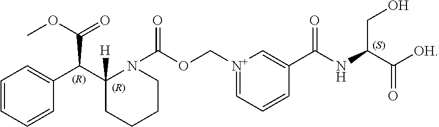

In another aspect, the composition prevents at least one methylphenidate-related adverse effect after oral, intranasal, and/or intravenous administration to a human or animal subject when compared to an equivalent molar amount of administered unconjugated d-methylphenidate.

In one embodiment, the conjugate can be an ionic salt, such as chloride, preferably serdexmethylphenidate chloride, having the following Formula II:

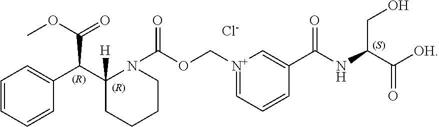

In some embodiments, compositions comprising serdexmethylphenidate may comprise up to about 10% by weight, alternatively up to about 5% by weight of methylphenidate active that is provided by sources other than the serdexmethylphenidate conjugate of the present technology, including but not limited to, other conjugates, unconjugated methylphenidate, methylphenidate-like stimulants, amphetamines, and amphetamine-like stimulants. In some embodiments, the conjugate compositions and formulations of the present technology do not contain unconjugated methylphenidate prior to administration to a human or animal patient or subject.

In some other embodiments, the d-methylphenidate active is derived from two sources, the serdexmethylphenidate conjugate and/or its pharmaceutically acceptable salts, and unconjugated methylphenidate and/or its pharmaceutically acceptable salts. The molar amount that each source contributes to the total molar dose of the unconjugated d-methylphenidate and the serdexmethylphenidate conjugate can vary from about 5% to about 95%, including, but not limited to, amounts of about 10%, about 20%, about 30%, about 40%, about 50%, about 60%, about 70%, about 80%, about 90%, or any amounts in between, in increments of about 0.5%, about 1%, about 2.5%, or about 5%.

In some further embodiments, the serdexmethylphenidate conjugate contributes a molar dose amount that is about 60%, alternatively about 70%, alternatively about 75%, alternatively about 80%, alternatively about 85%, alternatively about 90%, or alternatively about 95%, of the total combined molar dose of unconjugated d-methylphenidate and the serdexmethylphenidate conjugate, or any amounts in between, in increments of about 0.5%, about 1%, about 2%, about 2.5%, or 5%; and the unconjugated methylphenidate contributes about 40%, alternatively about 30%, alternatively about 25%, alternatively about 20%, alternatively about 15%, alternatively about 10%, or alternatively about 5% to the total molar dose, in increments of about 0.5%, about 1%, about 2%, about 2.5%, or 5%. It should also be appreciated by those skilled in the relevant art that, in some alternative embodiments, additional sources can contribute to the d-methylphenidate active, including but not limited to, other conjugates, unconjugated methylphenidate, methylphenidate-like stimulants, amphetamines, and amphetamine-like stimulants.

### Administration, Formulation and Advantages

The compositions comprising serdexmethylphenidate of the present technology can be administered orally and, upon administration, it is believed without being bound to any particular theory, releases the active d-methylphenidate, derivatives thereof or combinations thereof, after being hydrolyzed in the body. It is also believed, again without being bound to any particular theory, that the serdexmethylphenidate conjugates of the present technology can be easily recognized by physiological systems resulting in hydrolysis and release of d-methylphenidate after oral administration.

It has been surprisingly found that in some embodiments of the present technology, the serdexmethylphenidate conjugates of the present application can provide slower or delayed onset of certain adverse effects normally attributed to an equimolar amount of d-methylphenidate. In some embodiments, it has been further found that serdexmethylphenidate mitigates or substantially reduces the amount, frequency, and/or severity of certain adverse effects. These adverse effects include, but are not limited to, increased heartbeat, increased blood pressure, chest pain, fever, joint pain, skin rash, or hives, nausea, headache, vomiting, decreased appetite, xerostomia, anxiety, tics, hyperhidrosis, euphoria, and irritability

In another aspect, the serdexmethylphenidate conjugates reduces or prevents at least one methylphenidate-related adverse effect after oral, intranasal, and/or intravenous administration to a human or animal subject when compared to an equivalent molar amount of administered unconjugated d-methylphenidate.

In at least one embodiment, the serdexmethylphenidate conjugate of the present technology may alter the metabolic profile of d-methylphenidate, derivatives thereof or combinations thereof, by, for example, changing the amounts and/or ratio of d-methylphenidate and its metabolites, such as the inactive ritalinic acid within the human or animal body being exposed and/or treated with the serdexmethylphenidate of the present technology. The serdexmethylphenidate conjugate of the present technology, for example, may decrease the number and/or the amount of metabolites, including active, inactive, toxic or non-toxic metabolites, produced by unconjugated d-methylphenidate. Not wishing to be bound by any particular theory, it is believed that this change in metabolism may potentially alleviate certain side effects of metabolite(s), as well as potentially improve upon the safety profile of d-methylphenidate.

In another embodiment, the serdexmethylphenidate conjugate of the present technology may unexpectedly produce reduced interpatient and/or intrapatient variability of d-methylphenidate plasma concentrations. Not to be bound by any particular theory, it is believed that the reduction of interpatient or intrapatient variability of d-methylphenidate plasma concentrations may be due to either increased solubility or a modified metabolic pathway or a combination of both.

In yet another embodiment, the serdexmethylphenidate conjugate of the present technology is also believed to alter the metabolic pathway of the released d-methylphenidate when compared to unconjugated d-methylphenidate. It is further believed that in such an embodiment, the prodrug may decrease interpatient and/or intrapatient variability and/or reduce side effects associated with unconjugated d-methylphenidate or any of its metabolites. Common side effects of methylphenidate are nervousness, agitation, anxiety, and insomnia or drowsiness. Other common side effects are abdominal pain, weight loss, hypersensitivity, nausea, dizziness, palpitation, headache, dyskinesia, blood pressure, heartrate changes, tachycardia, angina, and cardiac arrhythmia, among others.

In a still further embodiment, the serdexmethylphenidate conjugate of the present technology is believed, without being bound to any particular theory, to exhibit an improved extended-release or extended-duration PK profile when compared to unconjugated d-methylphenidate when administered orally at equimolar doses.

Conventionally, d-methylphenidate has rewarding properties in terms of feeling pleasure and is prone to substance abuse because of its pharmacological similarity to cocaine and amphetamine. Oral abuse has been reported to lead to hallucinations, paranoia, euphoria, and delusional disorder. Oral abuse may subsequently escalate to intravenous and intranasal abuse. Euphoria has been reported after intravenous administration of d-methylphenidate. When administered intranasally the effect is found to be similar to intranasal use of amphetamines.

The serdexmethylphenidate conjugate, compositions and/or methods of the present technology are also believed to provide reduced potential for overdose, reduced potential for abuse and/or improve the characteristics of d-methylphenidate, derivatives thereof or combinations thereof with regard to toxicities or suboptimal release profiles in human or animal subjects. The serdexmethylphenidate conjugates of the present technology may produce reduced exposure to methylphenidate and as a result, have no or a substantially decreased pharmacological effect when compared to an equimolar dose of unconjugated methylphenidate administered through injection or intranasal routes of administration. The serdexmethylphenidate conjugates of the present technology may additionally or alternatively reduce or delay the rate of systemic d-methylphenidate absorption when compared to an equimolar dose of unconjugated methylphenidate administered through injection or intranasal routes of administration.

However, the serdexmethylphenidate conjugates of the present technology still release active d-methylphenidate into the circulation in amounts that provide one or more therapeutic effects when administered orally at equivalent or possibly even lower doses when compared to injection or intranasal routes of administration. Without wishing to be limited to the below theory, it is believed that overdose protection may occur due to the conjugates being exposed to different enzymes and/or metabolic pathways after oral administration in human or animal subjects, whereby the serdexmethylphenidate conjugate of the present technology is exposed to the gut and first-pass metabolism as opposed to exposure to enzymes or conditions in the circulation or mucosal membranes in the nose, which limits the ability of the d-methylphenidate, derivatives thereof or combinations thereof, from being released from the serdexmethylphenidate conjugate. Therefore, it is believed that abuse resistance, abuse deterrence, or lower abuse potential is provided by limiting the effectiveness of releasing d-methylphenidate from serdexmethylphenidate when administered via alternative routes. In some embodiments, the serdexmethylphenidate conjugate has route-specific bioavailability which may be a result of differential hydrolysis of the chemical linkage (i.e., a covalent linkage) between the d-methylphenidate moiety and the remainder of the serdexmethylphenidate conjugate following oral, intranasal, or intravenous administration in human or animal subjects. In yet another embodiment, the serdexmethylphenidate conjugate is envisioned not to hydrolyze or to hydrolyze at a reduced rate or to a limited extent via non-oral routes. As a result, in these embodiments, the serdexmethylphenidate conjugates are also believed to not generate high plasma or blood concentrations of released d-methylphenidate when injected or snorted in human or animal subjects as compared to free, unconjugated d-methylphenidate administered through these routes.

In some additional embodiments of the present technology, the AUC of d-methylphenidate is about 10% (or smaller) of the AUC of d-methylphenidate for unconjugated d-methylphenidate, when administered intravenously or intranasally at equimolar doses, for example about 50% to about 0.1%, alternatively from about 25% to about 0.1%, or alternatively from about 50% to about 1%, including, but not limited to, about 50%, about 40%, about 30%, about 20%, about 10%, about 1% or any amounts in between, in increments of about 0.5%, about 1%, about 2%, about 2.5%, about 5% or about 10%. In some embodiments, the mean peak methylphenidate exposure (Cₘₐₓ) can be reduced to about 20% of the Cₘₐₓ of unconjugated d-methylphenidate and the overall exposure to methylphenidate (AUCₗₐₛₜ and AUC_{inf}) can be reduced to about 10 to about 15%, preferably 10%, of the overall exposure of unconjugated methylphenidate after intravenous administration of serdexmethylphenidate in human or animal subjects when compared to an equimolar amount of unconjugated d-methylphenidate.

In further embodiments, the compositions of the present technology potentially reduce drug liking. Without being bound by theory, since d-methylphenidate is covalently bound in the conjugate, there is a slower of release of d-methylphenidate compared to an equimolar dose of unconjugated d-methylphenidate, which could lead to a reduced drug liking outcome.

It has been surprisingly found that in some embodiments of the present technology, the serdexmethylphenidate conjugates of the present technology provide a statistically significant reduction in peak and overall d-methylphenidate exposure with serdexmethylphenidate versus unconjugated d-methylphenidate when administered intravenously in a human at equimolar doses. The improved pharmacodynamics of serdexmethylphenidate resulted in meaningful statistically lower scores in the pharmacodynamic measures of “Drug Liking”, “Feeling High”, “Good Effects”, “Overall Drug Liking”, and “Take Drug Again” when compared to unconjugated d-methylphenidate.

In some embodiments, the serdexmethylphenidate conjugates of the present technology provide improvement across multiple abuse measures relative to unconjugated d-methylphenidate. For example, the “Take Drug Again” endpoint is lower with serdexmethylphenidate. The “Take Drug Again” measure may play an important role in the premarket assessment of abuse-deterrent technologies and/or abuse potential for predicting their performance in the real world for human subjects.

It is further believed, that the present technology provides a stimulant based treatment modality and dosage form for certain disorders requiring the stimulation of the CNS such as, attention-deficit hyperactivity disorder (ADHD), ADD (technically ADHD Predominantly Inattentive Type), autistic spectrum disorder, autism, Asperger's disorder, pervasive developmental disorder, sleep disorder, obesity, depression, bipolar disorder, eating disorder, binge eating disorder, chronic fatigue syndrome, schizophrenia, major depressive disorder narcolepsy, excessive daytime sleepiness (EDS), stimulant use disorder, cocaine dependence, or stimulant dependence. In at least one preferred embodiment, compositions comprising serdexmethylphenidate of the present technology can be used to treat attention-deficit/hyperactivity disorder (ADHD).

In some embodiments of the present technology, there are envisaged and provided pharmaceutical compositions for treating a disorder or condition requiring stimulation of the central nervous system comprising a serdexmethylphenidate conjugate having the following chemical formula:

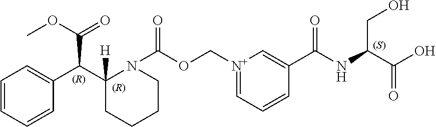

that, when administered at a therapeutically effective dose, may provide reduced and/or slower onset of side effects as compared to compositions comprising unconjugated d-methylphenidate without serdexmethylphenidate when administered at equimolar doses.

In certain embodiments, compositions of the present technology comprising serdexmethylphenidate can be used in neonatal, pediatric, adolescent, adult and/or geriatric subjects with ADHD that, when administered at a therapeutically effective dose, may provide minimized and/or reduced adverse events in terms of severity, frequency, and/or duration as compared to compositions comprising unconjugated d-methylphenidate administered at equimolar doses. For example, in some embodiments, the present compositions can be used for once-daily dosing with a potentially improved onset and a long duration of action attributes that may benefit neonatal, pediatric and/or adolescent subjects with ADHD.

The compositions comprising serdexmethylphenidate conjugate of the present technology can be formulated into dosage forms that include, but are not limited to sublingual, gummy, chewable tablet, rapidly dissolving tablet, orally disintegrating tablet, tablet, capsule, soft gel capsule, caplet, troche, lozenge, a gel, powder, suspension, syrup, solution, oral thin film (OTF), oral strip, rectal film, or suppository. In some embodiments, the dosage forms are to be administered orally. Preferred oral administration forms are capsule, tablet, caplet, solutions, or OTF. Suitable dosing vehicles of the present technology include, but are not limited to, water, citrate buffer, phosphate buffered saline (PBS), 10% Tween in water, and 50% PEG-400 in water, among others.

It should be understood that in addition to the ingredients mentioned above, the formulations of the present technology can include other suitable agents such as anti-adherents, antioxidants, binders, coatings, disintegrants, gel forming agents, fillers, flavors, colors, colorants, glidants, lubricants, preservatives, sorbents and sweeteners. Such antioxidants would be acceptable food additives, food ingredients or food colors, and could include vitamin E, carotene, BHT or other antioxidants.

Other compounds which may be included by admixture are, for example, medically inert ingredients, e.g., solid and liquid diluents, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulfates; and other therapeutically acceptable accessory ingredients, such as humectants, preservatives, buffers and antioxidants, which are known additives for such formulations.

The ingredients mentioned herein are not intended to be exhaustive, and one of skill in the art will be able to formulate suitable compositions using known or to be known ingredients.

Methylphenidate is being marketed in numerous dosage forms and at various dosage strengths either as a racemic mixture of d- and l-threo-methylphenidate or as a single d-threo-isomer (Table 1). Recommended daily doses depend on the dosage form, active ingredient (single isomer or racemic mixture) and individual subject or patient titration.

TABLE 1
Examples of marketed methylphenidate
dosage forms and dosage strengths.

| Active | Dosage | Dosage | Proprietary |
|---|---|---|---|
| Ingredient | Form | Strength(s) | Name(s) |
| methylphenidate | instant release | 5, 10, 20 mg | Ritalin ® |
| hydrochloride | tablet | | |
| dexmethylphenidate | instant release | 2.5, 5, | Focalin ® |
| hydrochloride | tablet | 10 mg | |
| methylphenidate | extended release | 10, 20 mg | Methylin ER ®, |
| hydrochloride | tablet | | Metadate ER ® |
| methylphenidate | extended release | 10, 18, 20, | Concerta ® |
| hydrochloride | tablet | 27, 36, | |
| | | 54 mg | |
| methylphenidate | chewable tablet | 2.5, 5, | Methylin |
| hydrochloride | | 10 mg | |
| methylphenidate | extended release | 10, 20, 30, | Ritalin LA ® |
| hydrochloride | capsules | 40 mg | |
| methylphenidate | extended release | 10, 20, 30, | Metadate CD ® |
| hydrochloride | capsules | 40, 50, | |
| | | 60 mg | |
| dexmethylphenidate | extended release | 5, 10, 15, | Focalin ® XR ® |
| hydrochloride | capsules | 20, 30, | |
| | | 40 mg | |
| methylphenidate | transdermal | 10, 15, 20, | Daytrana ® |
| | patch | 30 mg/9 h | |
| methylphenidate | oral solution | 5, 10 mg/ | Methylin ® |
| hydrochloride | | 5 mL | |

In some embodiments, doses of the serdexmethylphenidate conjugate of the present technology can be higher or lower than doses of unconjugated methylphenidate depending on their molecular weight, the respective weight-percentage of methylphenidate as part of the whole conjugate or conjugate salt, their bioavailability (with respect to released d-methylphenidate), and their pharmacokinetic profile of released d-MPH. Additional dose adjustments may be required depending on the isomer composition of the unconjugated methylphenidate reference dose. Therefore, dosages may be higher or lower than the dosages of free methylphenidate.

In further embodiments, weight amounts or doses of unconjugated or conjugated d-methylphenidate (serdexmethylphenidate), and any of their salt forms can be expressed as the molar equivalent weight amount or dose of any other compound or a salt thereof. For example, a dose of serdexmethylphenidate can alternatively be expressed as an equimolar dose of serdexmethylphenidate chloride, d-methylphenidate, or d-methylphenidate hydrochloride. A dose of d-methylphenidate hydrochloride can alternatively be expressed as an equimolar dose of d-methylphenidate, serdexmethylphenidate, or serdexmethylphenidate chloride. The general formula to calculate the molar equivalent dose of Compound 2 from the dose of Compound 1 is as follows:

$$Dose ( Compound 2 ) = Dose ( Compound 1 ) \times \frac{MW ( Compound 2 )}{MW ( Compound 1 )}$$

$$Dose ( Compound 1 ) = dose of Compound 1 ( in mass units )$$

$$Dose ( Compound 1 ) = dose of Compound 1 ( in mass units )$$

$$MW ( Compound 1 ) = molecular weight of Compound 1$$

$$MW ( Compound 2 ) = molecular weight of Compound 2$$

The following table lists the molecular weights of unconjugated d-methylphenidate and a salt form thereof, and serdexmethylphenidate and a salt form thereof.

| | Molecular Weight |
|---|---|
| Compound | (g/mol) |
| Serdexmethylphenidate | 500.53 |
| Serdexmethylphenidate chloride | 535.98 |
| d-methylphenidate | 233.31 |
| d-methylphenidate hydrochloride | 269.77 |

It is contemplated that daily dosing regimens for compositions of the present technology comprising serdexmethylphenidate include, but are not limited to, an amount of d-methylphenidate that is molar equivalent to a dose of d-methylphenidate from about 0.1 mg to about 500 mg per day, alternatively about 0.5 mg to about 480 mg per day, alternatively about 0.5 mg to about 450 mg per day, alternatively about 0.5 mg to about 400 mg per day, alternatively about 0.5 mg to about 360 mg per day, alternatively about 0.5 mg to about 350 mg per day, alternatively about 0.5 mg to about 300 mg per day, alternatively about 1 mg to about 250 mg per day, alternatively about 5 mg to about 240 mg per day, alternatively about 1 mg to about 100 mg per day, alternatively about 5 mg to about 80 mg per day, alternatively about 10 mg to about 40 mg per day, alternatively about 10 mg to 200 mg per day, alternatively about 10 mg to about 180 mg per day, alternatively about 20 mg to about 120 mg per day, alternatively about 20 mg to about 150 mg per day, alternatively about 30 mg to about 100 mg per day, alternatively about 40 mg to about 80 mg per day, alternatively about 50 mg to about 70 mg per day, alternatively about 20 mg to about 40 mg per day, alternatively about 20 mg to about 60 mg per day, a alternatively about 10 mg to about 50 mg per day, alternatively about 20 mg per day, alternatively about 30 mg per day, alternatively about 40 mg per day, alternatively about 60 mg per day, alternatively about 80 mg per day, alternatively about 100 mg per day, or alternatively about 120 mg per day. It is also contemplated that compositions comprising serdexmethylphenidate would have a dosing regimen of one time a day, alternatively, every other day, alternatively two times a day, alternatively four times a day or more.

It is contemplated that some of the formulations of the present technology would be provided in a unit dose form. “Unit dose form” herein means a single entity of a solid therapeutic dosage form (e.g., 1 capsule, 1 tablet, 1 caplet, etc.) or a single volume dispensed from a non-solid dosage form (e.g., 5 mL of a liquid or syrup, suspension, slurry, etc.). Such a unit dose form can be from about 0.5 mg to about 400 mg, alternatively from about 0.1 mg to about 300 mg, about 0.5 mg to about 300 mg, alternatively about 1 mg to about 250 mg, alternatively about 5 mg to about 240 mg, alternatively about 1 mg to about 100 mg, alternatively about 5 mg to about 80 mg, alternatively about 10 mg to about 40 mg, alternatively about 10 mg to 200 mg, alternatively about 10 mg to about 180 mg, alternatively about 20 mg to about 120 mg, alternatively about 20 mg to about 150 mg, alternatively about 30 mg to about 100 mg, alternatively about 40 mg to about 80 mg, alternatively about 50 mg to about 70 mg, alternatively about 20 mg to about 40 mg, alternatively about 20 mg to about 60 mg, a alternatively about 10 mg to about 50 mg, alternatively about 20 mg, alternatively about 40 mg, alternatively about 60 mg, alternatively about 80 mg, alternatively about 100 mg, or alternatively about 120 mg. The present technology provides for dosage forms formulated as a single therapy or as a combination therapy.

Doses of compositions of the present technology that comprise serdexmethylphenidate and unconjugated d-methylphenidate may be provided in fixed molar dose ratios in the following format: “mol-% of serdexmethylphenidate conjugate/mol-% of the unconjugated d-methylphenidate active”. Fixed molar dose ratios can range from about 95% to about 5% for the serdexmethylphenidate conjugate and about 5% to about 95% for unconjugated d-methylphenidate. In some embodiments, the dose ratios may be about 95%/5%, alternatively about 90%/10%, alternatively about 85%/15%, alternatively about 80%/20%, alternatively about 75%/25%, alternatively 70%/30%, alternatively about 65%/35%, alternatively about 60%/40%, or alternatively about 50%/50% serdexmethylphenidate/d-methylphenidate. In some embodiments, at a fixed molar dose ratio, compositions comprising serdexmethylphenidate and unconjugated d-methylphenidate are dose proportional across a wide range of doses. For example, compositions comprising serdexmethylphenidate and unconjugated d-methylphenidate at a fixed molar dose ratio of about 70%/30%, at dosage strengths for serdexmethylphenidate chloride/d-methylphenidate hydrochloride of 28/6 mg, 42/9 mg, and 56/12 mg, which are equimolar to 20 mg, 30 mg, and 40 mg of d-methylphenidate hydrochloride, respectively, provide d-methylphenidate plasma concentration that are proportional to the amount of total d-methylphenidate active in the dose. In some embodiments, the dosage strengths of compositions comprising serdexmethylphenidate and unconjugated d-methylphenidate at a fixed molar dose ratio of about 70%/30% of serdexmethylphenidate chloride/d-methylphenidate hydrochloride may be 7/1.5 mg, 14/3 mg, 21/4.5 mg, 35/7.5 mg, 49/10.5 mg, 63/13.5 mg, 70/15 mg, 77/16.5 mg, 84/18 mg, 91/19.5 mg, 98/21 mg, 105/22.5 mg, and 110/24 mg. In further embodiments, the dosage strengths of compositions comprising serdexmethylphenidate chloride and unconjugated d-methylphenidate hydrochloride at a fixed molar dose ratio of about 70%/30% of serdexmethylphenidate/d-methylphenidate may be 6.5/1.3 mg, 13.1/2.6 mg, 19.6/3.9 mg, 26.1/5.2 mg, 32.7/6.5 mg, 39.2/7.8 mg, 45.8/9.1 mg, 52.3/10.4 mg, 58.8/11.7 mg, 65.4/13 mg, 71.9/14.3 mg, 78.4/15.6 mg, 85/16.9 mg, 91.5/18.2 mg, 98.1/19.5 mg, and 102.7/20.8 mg.

In yet further embodiments, the dosage strengths of compositions comprising serdexmethylphenidate and unconjugated d-methylphenidate at a fixed molar dose ratio of about 90%/10% of serdexmethylphenidate chloride/d-methylphenidate hydrochloride may be 42.8/2.31 mg, 64.3/3.47 mg, 75/4.05 mg, 85.7/4.63 mg, 107.1/5.78 mg, 128.5/6.94 mg, 139.2/7.52 mg, and 149.9/8.09 mg. In some embodiments, the dosage strengths of compositions comprising serdexmethylphenidate chloride and unconjugated d-methylphenidate hydrochloride at a fixed molar dose ratio of about 90%/10% of serdexmethylphenidate/d-methylphenidate may be 40/2 mg, 60/3 mg, 70/3.5 mg, 80/4 mg, 100/5 mg, 110/5.5 mg, 120/6 mg, 130/6.5 mg, and 140/7 mg.

In at least some embodiments, the compositions comprising the serdexmethylphenidate conjugates of the present technology have one or more advantages, including, but not limited to, providing a more gradual rise in plasma concentration of d-methylphenidate prior to Tₘₐₓ and/or a more gradual decrease in plasma concentrations after Tₘₐₓ, which may provide a reduced or improved side effect profile or reduced adverse effects, formation of less potentially toxic metabolites, formation of less inactive metabolites, reduced acute tolerance, reduced drug abuse potential and/or reduced interpatient and/or intrapatient variability in plasma concentrations as compared to unconjugated d-methylphenidate. In addition, at least some embodiments of the compositions of the present technology exhibit dose-proportionality, allowing greater predictability in dosing regimens.

### Synthetic Schemes

General synthetic schemes for preparing prodrugs of d-methylphenidate are disclosed in U.S. Pat. No. 9,079,928, which is herein incorporated by reference. One or more protecting groups may be attached to any reactive functional groups that may interfere with the coupling to d-methylphenidate. Any suitable protecting group may be used depending on the type of functional group and reaction conditions. Some protecting groups suitable for use in the present technology include, but are not limited to, acetyl (Ac), tert-butyl (tBu), tert-butyoxycarbonyl (Boc), benzyloxycarbonyl (Cbz), p-methoxybenzylcarbonyl (Moz), 9-fluorenylmethyloxycarbonyl (Fmoc), benzyl (Bn), p-methoxybenzyl (PMB), 3,4 dimethoxybenzyl (DMPM), p-methozyphenyl (PMP), tosyl (Ts), or amides (like acetamides, phthalimides, and the like).

In other embodiments, a base may be required at any step in the synthetic scheme of preparing the prodrug of d-methylphenidate. Suitable bases include, but are not limited to, 4-methylmorpholine (NMM), 4-(dimethylamino)pyridine (DMAP), N,N-diisopropylethylamine (DIPEA), lithium bis(trimethylsilyl)amide, lithium diisopropylamide (LDA), any alkali metal tert.-butoxide (e.g., potassium tert.-butoxide), any alkali metal hydride (e.g., sodium hydride), any alkali metal alkoxide (e.g., sodium methoxide), triethylamine (Et3N or TEA) or any other tertiary amine.

Suitable solvents that can be used for any reaction at any step in the synthetic scheme of preparing the prodrug of d-methylphenidate include, but are not limited to, acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, 2,6-lutidine, methanol, methyl isobutyl ketone (MIBK), methyl tent.-butyl ether (MTBE), isopropanol (IPA), isopropyl acetate (IPAc), diisopropyl ether, tetrahydrofuran, toluene, xylene or water.

In some embodiments, an acid may be used to remove certain protecting groups. Suitable acids include, but are not limited to, hydrochloric acid, hydrobromic acid, hydrofluoric acid, hydroiodic acid, sulfuric acid, phosphoric acid, trifluoroacetic acid, acetic acid, citric acid, methanesulfonic acid, p-toluenesulfonic acid and nitric acid. For certain other protecting groups, a catalytic hydrogenation may be used, e.g., palladium on charcoal in the presence of hydrogen gas.

In some embodiments, an anion exchange medium, anion exchange resin, strong or weak anion exchanger including but not limited to Dowex® 1×8 chloride (available from Dow Chemical Co, Midland, Mich.) may be used to replace anionic counter ions of the cationic conjugate with a specific new counter anion such as a chloride ion.

Synthetic Process for Making Serdexmethylphenidate

1. Synthesis of nicotinoyl-Ser(tBu)-OtBu

In one embodiment, the nicotinoyl-Ser(tBu)-OtBu precursor is prepared according to Scheme 1.

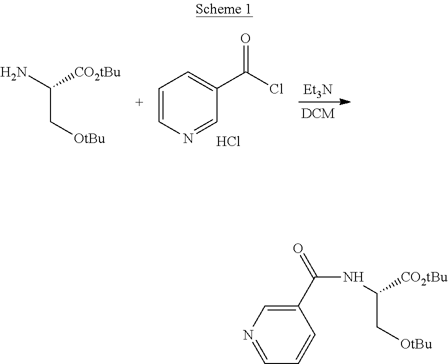 [SMILES: "Et3N" ; CC(C)(C)OC[C@H](N)C(=O)OC(C)(C)C ; O=C(Cl)c1cccnc1 ; Cl ; CC(C)(C)OC[C@H](NC(=O)c1cccnc1)C(=O)OC(C)(C)C]

2. Synthesis of d-MPH-N-CO₂CH₂—Cl

In one embodiment, the d-MPH-N-CO₂CH₂—Cl precursor can be prepared according to Scheme 2.

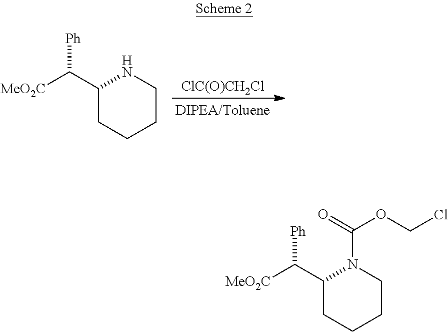

In an alternate embodiment, d-MPH-N-CO₂CH₂—Cl can be prepared according to Scheme 3.

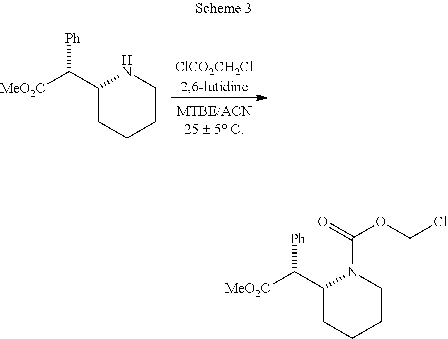

3. Preparation of Protected Serdexmethylphenidate

In one embodiment, the protected serdexmethylphenidate intermediate can be prepared as shown in Scheme 4.

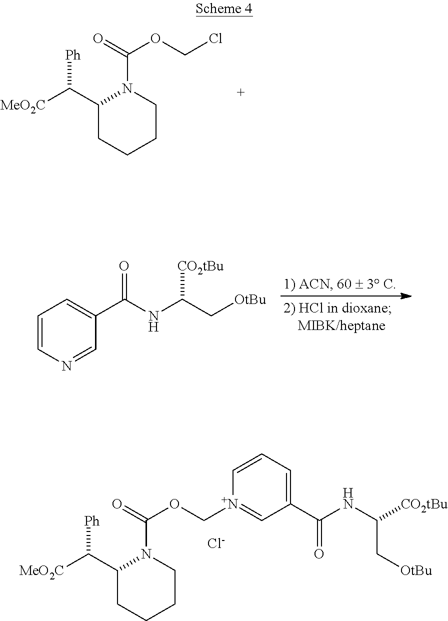

In an alternate embodiment, the protected serdexmethylphenidate intermediate can be prepared according to Scheme 5.

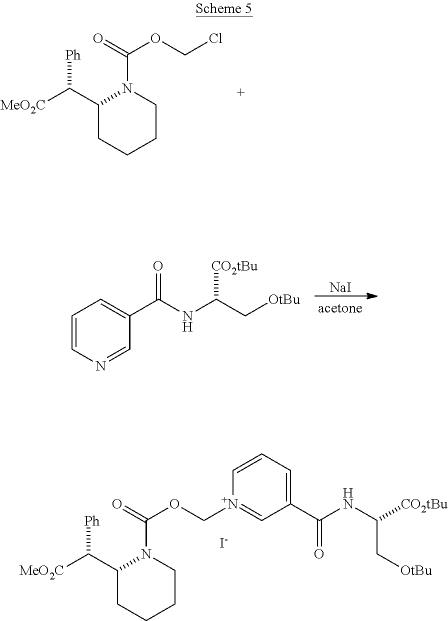

4. Deprotection of Protected Serdexmethylphenidate

In one embodiment, serdexmethylphenidate chloride can be prepared according to Scheme 6.

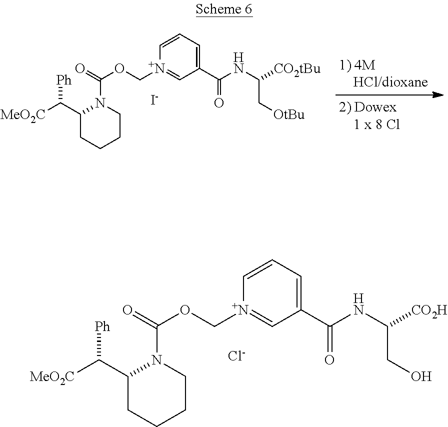

In an alternate embodiment, serdexmethylphenidate chloride can be prepared according to Scheme 7.

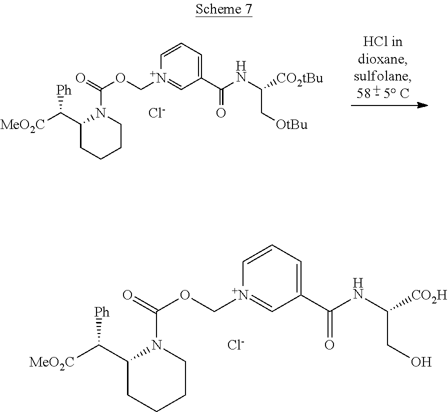 [SMILES: COC(=O)[C@H](c1ccccc1)[C@H]1CCCCN1C(=O)OC[n+]1cccc(C(=O)N[C@@H](COC(C)(C)C)C(=O)OC(C)(C)C)c1 ; [Cl-] ; COC(=O)[C@H](c1ccccc1)[C@H]1CCCCN1C(=O)OC[n+]1cccc(C(=O)N[C@@H](CO)C(=O)O)c1 ; [Cl-]]

Following deprotection (for example, but not limited to, deprotection methods as illustrated by Scheme 6 or Scheme 7) of a protected serdexmethylphenidate intermediate (for example, but not limited to, the serdexmethylphenidate intermediate prepared according to Scheme 4 or Scheme 5) , crude serdexmethylphenidate can be purified by several methods, including, but not limited to, the method according to Scheme 8.

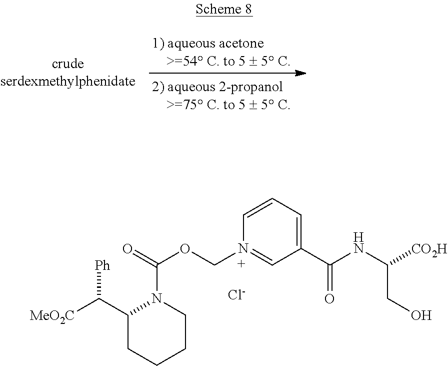

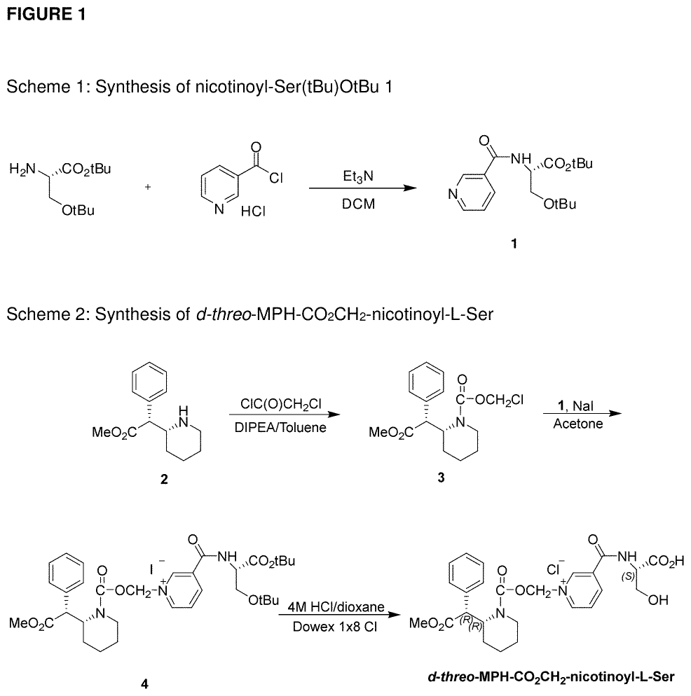

An alternative embodiment for preparing serdexmethylphenidate is shown in FIG. 1.

Novel intermediates are produced during the process of synthesizing serdexmethylphenidate (i.e., process intermediates). These process intermediates may be isolated or form in situ, and include, but are not limited to, 3-(((S)-2-(tert-butoxy)-1-carboxyethyl)carbamoyl)-1-((((R)-2-((R)-2-methoxy-2-oxo-1-phenylethyl)piperidine-1-carbonyl)oxy)methyl)pyridin-1-ium; tert-butyl O-(tert-butyl)-N-nicotinoyl-L-serinate; chloromethyl (R)-2-((R)-2-methoxy-2-oxo-1-phenylethyl)piperidine-1-carboxylate; and 3-(((S)-1,3-di-tert-butoxy-1-oxopropan-2-yl)carbamoyl)-1-((((R)-2-((R)-2-methoxy-2-oxo-1-phenylethyl)piperidine-1-carbonyl)oxy)methyl)pyridin-1-ium.

Novel metabolites and/or novel degradants are produced during the breakdown of serdexmethylphenidate in vitro and/or in vivo. These metabolites and/or degradants include, but are not limited to, 1-((((R)-2-((R)-carboxy(phenyl)methyl)piperidine-1-carbonyl)oxy)methyl)-3-(((S)-1-carboxy-2-hydroxyethyl)carbamoyl)pyridin-1-ium; and 3-carboxy-1-((((R)-2-((R)-2-methoxy-2-oxo-1-phenylethyl)piperidine-1-carbonyl)oxy)methyl)pyridin-1-ium; nicotinic acid (niacin); and nicotinoyl-L-serine.

In certain embodiments of synthesizing serdexmethylphenidate other compounds may be produced including, but not limited to, dichloromethyl (R)-2-((R)-2-methoxy-2-oxo-1-phenylethyl)piperidine-1-carboxylate; 3-((1-carboxy-2-(((1-((((R)-2-((R)-2-methoxy-2-oxo-1-phenylethyl)piperidine-1-carbonyl)oxy)methyl)pyridin-1-ium-3-carbonyl)-L-seryl)oxy)ethyl)carbamoyl)-1-((((S)-2-((S)-2-methoxy-2-oxo-1-phenylethyl)piperidine-1-carbonyl)oxy)methyl)pyridin-1-ium; N,N-diethyl-N-((((R)-2-((R)-2-methoxy-2-oxo-1-phenylethyl)piperidine-1-carbonyl)oxy)methyl)ethanaminium; 1-((((R)-2-((R)-2-methoxy-2-oxo-1-phenylethyl)piperidine-1-carbonyl)oxy)methyl)-2,6-dimethylpyridin-1-ium; (((S)-1,3-di-tert-butoxy-1-oxopropan-2-yl)amino)methyl (R)-2-((R)-2-methoxy-2-oxo-1-phenylethyl)piperidine-1-carboxylate; ((R)-2-((R)-2-methoxy-2-oxo-1-phenylethyl)piperidin-1-yl)methyl (R)-2-((R)-2-methoxy-2-oxo-1-phenylethyl)piperidine-1-carboxylate; 3-(((R)-1-carboxy-2-chloroethyl)carbamoyl)-1-((((R)-2-((R)-2-methoxy-2-oxo-1-phenylethyl)piperidine-1-carbonyl)oxy)methyl)pyridin-1-ium; and 3-(((S)-3 -hydroxy-1-isopropoxy-1-oxopropan-2-yl)carbamoyl)-1-((((R)-2-((R)-2-methoxy-2-oxo-1-phenylethyl)piperidine-1-carbonyl)oxy)methyl)pyridin-1-ium.

Structural examples of process intermediates, degradants, and/or metabolites are listed in Table 1A.

TABLE 1A

| Structure | Chemical Name |
|---|---|
| 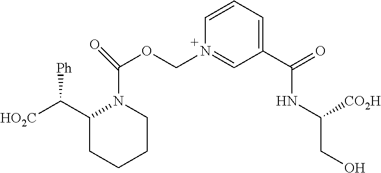 | 1-((((R)-2-((R)-carboxy(phenyl)methyl)piperidine- 1-carbonyl)oxy)methyl)-3-(((S)-1-carboxy-2- hydroxyethyl)carbamoyl)pyridin-1-ium |
| 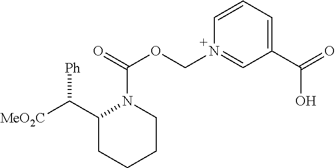 | 3-carboxy-1-((((R)-2-((R)-2-methoxy-2-oxo-1- phenylethyl)piperidine-1- carbonyl)oxy)methyl)pyridin-1-ium |
| 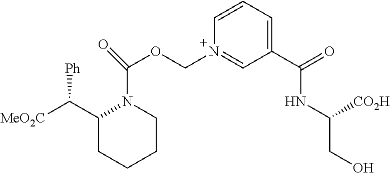 | 3-(((S)-2-(tert-butoxy)-1-carboxyethyl)carbamoyl)- 1-((((R)-2-((R)-2-methoxy-2-oxo-1- phenylethyl)piperidine-1- carbonyl)oxy)methyl)pyridin-1-ium |
| 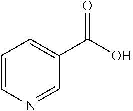 | Nicotinic acid (niacin) |
| 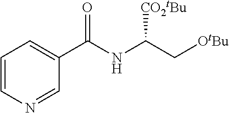 [SMILES: CC(C)(C)OC[C@H](NC(=O)c1cccnc1)C(=O)OC(C)(C)C] | tert-butyl O-(tert-butyl)-N-nicotinoyl-L-serinate |
| 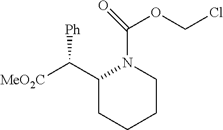 | chloromethyl (R)-2-((R)-2-methoxy-2-oxo-1- phenylethyl)piperidine-1-carboxylate |
| 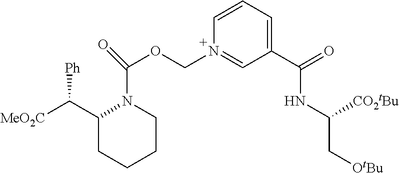 | 3-(((S)-1,3-di-tert-butoxy-1-oxopropan-2- yl)carbamoyl)-1-((((R)-2-((R)-2-methoxy-2-oxo-1- phenylethyl)piperidine-1- carbonyl)oxy)methyl)pyridin-1-ium |
| 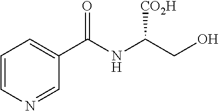 [SMILES: O=C(N[C@@H](CO)C(=O)O)c1cccnc1] | nicotinoyl-L-serine |

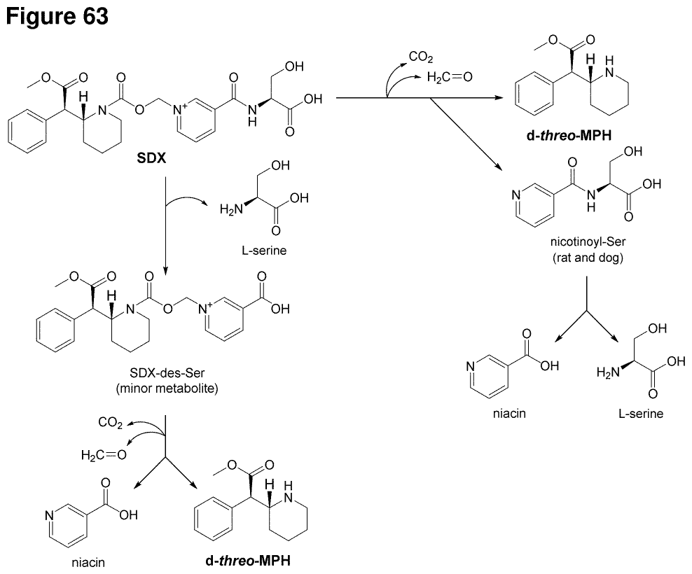

A proposed metabolic pathway of serdexmethylphenidate is shown in FIG. 63.

### Pharmaceutical Kits

The present technology provides one or more pharmaceutical kits for the treatment or prevention of indications in a subject including ADHD, eating disorder, binge eating disorder, obesity, narcolepsy, chronic fatigue, sleep disorder, EDS, substance use disorder, cocaine addiction, or drug withdrawal symptoms in a human or animal subject that, when the serdexmethylphenidate conjugate of the present technology is administered at a therapeutically effective dose, it may provide reduced and/or slower onset of side effects as compared to compositions comprising unconjugated d-methylphenidate administered at equimolar doses. As used herein the term animal is used in the veterinary sense and does not include humans. Suitable human subjects include neonatal subjects, pediatric subjects, adolescent subjects, adult subjects, geriatric subjects, elderly subjects, and normative subjects. In some embodiments, the kit comprises a specific amount of individual doses in a package, each dose containing a pharmaceutically and/or therapeutically effective amount of a composition comprising serdexmethylphenidate conjugate of the present technology alone or in combination with other additives, adjuvants, excipients, and the like. The kit can further include instructions for use of the kit, wherein the instructions for use of the kit may further comprise methods for treating or preventing any of the indications selected from the group consisting of ADHD, eating disorder, binge eating disorder, obesity, narcolepsy, chronic fatigue, sleep disorder, EDS, substance use disorder, cocaine addiction, or drug withdrawal symptoms in a subject. The kit can further include instructions for dose titration and/or instructions for prevention or discouragement of abuse and/or tampering.

In some embodiments, the kit comprises oral thin films or strips comprising the composition comprising serdexmethylphenidate. In some other embodiments, the kit comprises one or more blister packs containing the composition comprising serdexmethylphenidate. In yet further embodiments, the kit comprises a bulk bottle comprising the composition comprising serdexmethylphenidate.

The specified amount of individual doses may be from about 1 to about 100 individual dosages, alternatively from about 1 to about 60 individual dosages, alternatively from about 10 to about 30 individual dosages, including, about 1, about 2, about 5, about 7, about 10, about 14, about 15, about 20, about 21, about 25, about 30, about 35, about 40, about 45, about 50, about 55, about 60, about 70, about 80, or about 100, and include any additional increments thereof, for example, about 1, about 2, about 5, about 10 and multiplied factors thereof, (e.g., about x1, about x2, about x2.5, about x5, about x10, or about x100, etc.).

It will be appreciated by one skilled in the art that, in some embodiments, the kit may include individual doses that have different dosage amounts. In some embodiments, the kit of the present technology may include graduated individual doses (i.e., dose amounts that increase or decrease over a period of time), and/or a graduated dosing regimen, and instructions for use. In some other embodiments, the pharmaceutical kit can comprise at least two sets of individual doses in a package, each set having a specified amount of individual doses, and instructions for use. In one set of doses, each individual dose can comprise a composition comprising unconjugated d-methylphenidate, and in a second set of doses, each individual dose can comprise a composition comprising serdexmethylphenidate. The at least two combined individual doses of the at least two sets of doses are therapeutically effective. Kits having at least two sets of individual doses of either unconjugated methylphenidate or serdexmethylphenidate or both may be useful to optimize the ratio and dosages of serdexmethylphenidate and unconjugated methylphenidate for individual titration of the subject with respect to duration of action, tolerability, severity of disorder, and/or dose response. Such kits may contain instructions for use instructing that the subject be administered or switched to different doses from the first set or second set, or both first and second set, comprising compositions comprising different doses of unconjugated methylphenidate and serdexmethylphenidate depending on the subject's need of stimulant treatment, tolerability, and/or duration of action.

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

### Example 1: Dose-Proportionality and Steady-State Study

A study was conducted in humans to assess the pharmacokinetics (PK) and dose-proportionality of three different doses of d-methylphenidate hydrochloride/serdexmethylphenidate chloride at a 30%/70% fixed molar dose ratio after oral administration under fasted conditions. The steady-state PK after administration of the highest clinical daily dose was also assessed. The three different doses were 6/28 mg, 9/42 mg, and 12/56 mg and contained combined total doses that are equimolar to 20 mg, 30 mg, and 40 mg d-methylphenidate hydrochloride, respectively. Twenty-four (24) healthy adults were enrolled in this Phase 1, open-label, randomized, single-dose, 3-treatment, 3-period crossover study. Following the crossover single-dose phase, all subjects received 4 doses of 12/56 mg/day of d-methylphenidate hydrochloride/serdexmethylphenidate chloride q24h for evaluating the steady-state PK. During the single-dose phase, plasma samples were collected from pre-dose through 72 hours post-dose, with denser sampling in the first 3 hours post-dose.

### Pharmacokinetic Sampling in the Single Dose Treatment Phase:

Relative to each of the 3 doses of study drug (Days 1, 5, and 9), at pre-dose (0 hour; within 1 hour prior to dosing), and at 0.5, 1, 1.5, 2, 2.5, 3, 5, 7, 9, 12, 13, 24, 36, 48, 60 and 72 hours ±5 minutes post-dose.

### Pharmacokinetic Sampling in the Multiple Dose Treatment Phase:

After the 1^{st} dose of study drug (Day 13), blood samples for PK were collected at pre-dose (0 hour; within 1 hour prior to dosing), and at 0.5, 1, 1.5, 2, 2.5, 3, 5, 7, 9, 12, 13, and 24 hours ±5 minutes post-dose. The 24-hr post-dose PK sample was taken before administration of the 2^{nd} dose of study drug.

After the 2^{nd} dose of study drug (Day 14), blood samples for PK were collected at 2, 8 and 24 hours ±5 minutes post-dose. The 24-hr post-dose PK sample was taken before administration of the 3^{rd} dose of study drug.

After the 3^{rd} dose of study drug (Day 15), blood samples for PK were collected at 2, 8 and 24 hours ±5 minutes post-dose. The 24-hr post-dose PK sample was taken before administration of the 4^{th} dose of study drug.

After the last dose (4^{th} dose) of study drug (Day 16), blood samples for PK will be at 0.5, 1, 1.5, 2, 2.5, 3, 5, 7, 9, 12, 13, 24, 36, 48, 60 and 72 hours ±5 minutes post-dose.

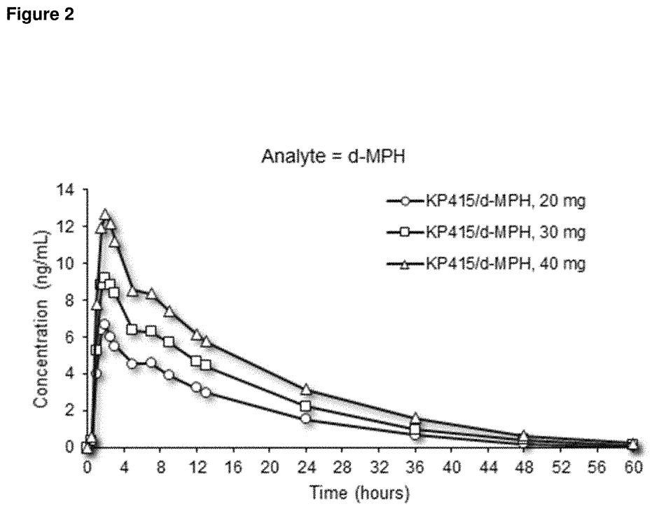

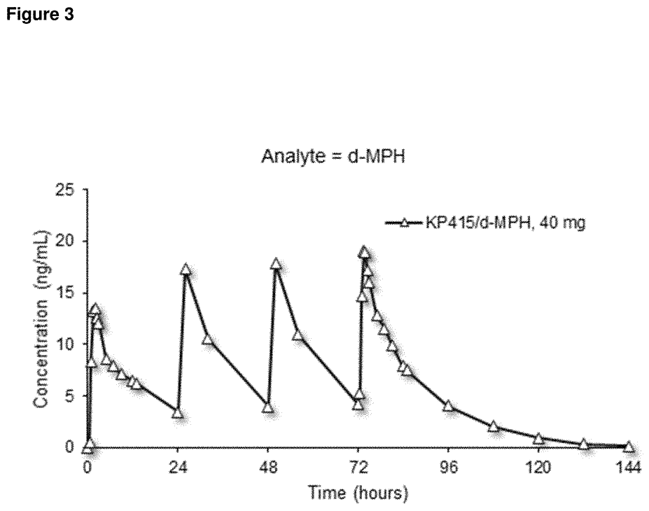

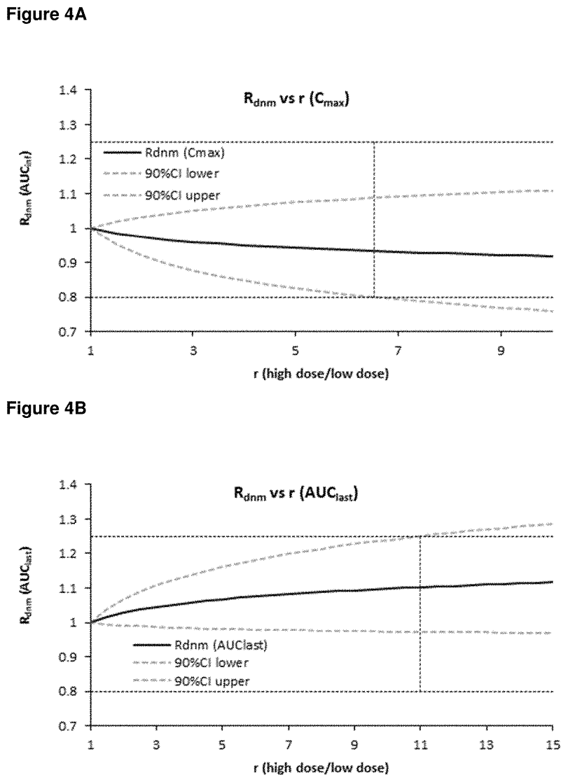

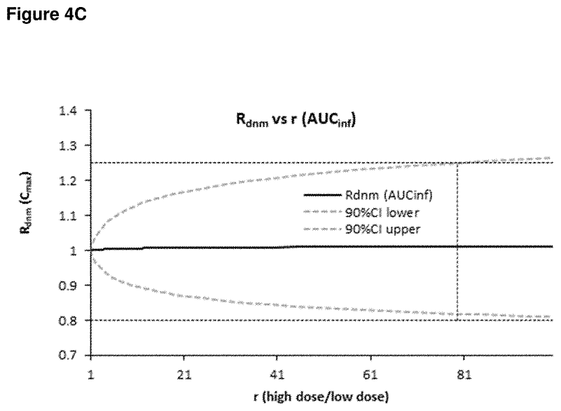

Results: After single-dose administration, d-methylphenidate plasma concentrations increased rapidly for all dosage strengths, with peak plasma concentrations (Cₘₐₓ) of 7.15, 9.88, and 13.8 ng/mL for the 6/28 mg, 9/42 mg, and 12/56 mg doses of d-methylphenidate hydrochloride/serdexmethylphenidate chloride, respectively. Plasma concentrations decreased gradually after Cₘₐₓ, with appreciable concentrations still apparent at 13 hours post-dose. FIG. 2 shows the d-methylphenidate plasma-concentration profiles for the three dose mixtures. As shown in FIG. 2, d-methylphenidate hydrochloride/serdexmethylphenidate chloride produces dose proportional increases in the rate and extent of d-methylphenidate exposure across the range of doses tested for Cₘₐₓ, AUCₗₐₛₜ, and AUC_{inf}, respectively. Analyses using a prespecified power analysis indicated that the compositions were dose-proportional across a 6.5-, 11.1-, and 82.7-fold range of doses for Cₘₐₓ, AUCₗₐₛₜ, and AUC_{inf}, respectively (FIG. 4). FIG. 3 shows the plasma concentration-time profile for the multiple-dose phase. In the multiple-dose phase, steady-state plasma concentrations are achieved after Dose 2 prior to Dose 3, as shown in FIG. 3.

This study demonstrates that d-methylphenidate hydrochloride/serdexmethylphenidate chloride has the potential to provide a rapid onset and extended duration of therapeutic benefit with predictable d-methylphenidate exposure during titration and maintenance.

### Dose Proportionality

TABLE 2
Mean PK parameters of d-methylphenidate released from
d-methylphenidate hydrochloride/
serdexmethylphenidate chloride 6/28, 9/42, and 12/56 mg.

| | | Cₘₐₓ | | AUCₗₐₛₜ | AUC_{inf} | |
|---|---|---|---|---|---|---|
| | | (ng/ | Tₘₐₓ | (ng*h/ | (ng*h/ | T_{1/2} |
| Treatment | Statistic | mL) | (h) | mL) | mL) | (h) |
| A | N | 23 | 23 | 23 | 23 | 23 |
| | Mean | 7.2 | 2.7 | 97.2 | 102.4 | 9.8 |
| | SD | 2.2 | 2.3 | 28.8 | 27.9 | 3.3 |
| B | N | 23 | 23 | 23 | 23 | 23 |
| | Mean | 9.9 | 2.6 | 142.5 | 148.6 | 10.3 |
| | SD | 2.9 | 1.8 | 41.2 | 40.9 | 3.6 |
| C | N | 23 | 23 | 23 | 23 | 23 |
| | Mean | 13.8 | 2.2 | 199.8 | 206.0 | 10.8 |
| | SD | 3.8 | 1.2 | 57.3 | 57.3 | 3.1 |

N = analysis sample size
SD = standard deviation
A = d-methylphenidate hydrochloride/serdexmethylphenidate chloride, 6/28 mg
B = d-methylphenidate hydrochloride/serdexmethylphenidate chloride, 9/42 mg
C = d-methylphenidate hydrochloride/serdexmethylphenidate chloride, 12/56 mg

TABLE 3
Dose range for predicted proportionality of d-
methylphenidate released from d-methylphenidate
hydrochloride/serdexmethylphenidate chloride:

| Parameter | Rho1 |
|---|---|
| Cₘₐₓ | 6.5 |
| AUCₗₐₛₜ | 11.1 |
| AUC_{inf} | 82.7 |

rho1 (ρ₁) = maximal predicted dose ratio for definitive proportionality

### Steady State (Multiple Dose PK)

TABLE 4
Mean PK parameters for d-methylphenidate released
from d-methylphenidate hydrochloride/
serdexmethylphenidate chloride, 12/56 mg after
single and multiple oral doses:

| Day | | Cₘₐₓ | | AUC_{0-24 h} | AUCₗₐₛₜ | AUC_{inf} | |
|---|---|---|---|---|---|---|---|
| of | | (ng/ | Tₘₐₓ | (ng*h/ | (ng*h/ | (ng*h/ | T_{1/2} |
| Dosing | Statistic | mL) | (h) | mL) | mL) | mL) | (h) |
| Day 1 | N | 23 | 23 | 23 | 23 | | |
| | Mean | 14.9 | 2.0 | 159.3 | 159.1 | | |
| | SD | 4.0 | 0.5 | 38.4 | 38.3 | | |
| Day 4 | N | 23 | 23 | 23 | 23 | 22 | 22 |
| | Mean | 20.0 | 1.8 | 215.4 | 280.5 | 291.7 | 9.8 |
| | SD | 4.7 | 0.44 | 49.4 | 69.8 | 69.18 | 2.5 |

N = analysis sample size
SD = standard deviation

TABLE 5
Mean PK parameters for serdexmethylphenidate after single
and multiple oral doses of d-methylphenidate hydrochloride/
serdexmethylphenidate chloride, 12/56 mg:

| Day | | Cₘₐₓ | | AUC_{0-24 h} | AUCₗₐₛₜ | AUC_{inf} | |
|---|---|---|---|---|---|---|---|
| of | | (ng/ | Tₘₐₓ | (ng*h/ | (ng*h/ | (ng*h/ | T_{1/2} |
| Dosing | Statistic | mL) | (h) | mL) | mL) | mL) | (h) |
| Day 1 | N | 23 | 23 | 23 | 23 | | |
| | Mean | 41.8 | 2.18 | 256.7 | 256.7 | | |
| | SD | 23.5 | 1.07 | 108.5 | 108.5 | | |
| Day 4 | N | 23 | 23 | 23 | 23 | 23 | 23 |
| | Mean | 41.7 | 1.8 | 241.2 | 246.9 | 248.6 | 5.5 |
| | SD | 38.0 | 1.0 | 159.9 | 161.0 | 160.8 | 1.8 |

N = analysis sample size
SD = standard deviation

TABLE 6
Accumulation of d-methylphenidate and serdexmethylphenidate
from Day 1 to Day 4 after oral doses of d-methylphenidate
hydrochloride/serdexmethylphenidate chloride, 12/56 mg:

| | d-Methylphenidate | | Serdexmethylphenidate | |
|---|---|---|---|---|
| Statistic | AR Cₘₐₓ | AR AUC_{0-24 h} | AR Cₘₐₓ | AR AUC_{0-24 h} |
| N | 23 | 23 | 23 | 23 |
| Mean | 1.37 | 1.37 | 1.00 | 0.92 |
| SD | 0.21 | 0.15 | 0.57 | 0.30 |

AR = accumulation ratio (Day 4/Day 1)
N = analysis sample size
SD = standard deviation

### Example 1A: Pharmacokinetics Study of SDX in Children and Adolescents

A single-dose, single period study was conducted to assess the pharmacokinetics (PK) of d-methylphenidate and serdexmethylphenidate (SDX) orally administered to children (6 to 12 years) and adolescents (13 to 17 years) with ADHD. The effect of body weight on the PK properties was also assessed. Following a standardized meal, subjects were administered d-methylphenidate hydrochloride/serdexmethylphenidate chloride at a 30%/70% fixed molar dose ratio. Eligible subjects (N=30) received treatments that were stratified into 3 age and 2 dose groups, whereby 6 to 8 year-olds (Cohort 1, n=10) received 6/28 mg d-methylphenidate hydrochloride/serdexmethylphenidate chloride, 9 to 12 year-olds (Cohort 2, n=10) received 12/56 mg d-methylphenidate hydrochloride/serdexmethylphenidate chloride, and 13 to 17 year-olds (Cohort 3, total n=10) received either 6/28 mg (n=5) or 12/56 mg (n=5) d-methylphenidate hydrochloride/serdexmethylphenidate chloride. The 6/28 mg and 12/56 mg doses of d-methylphenidate hydrochloride/serdexmethylphenidate chloride contained the same molar d-methylphenidate as 20 and 40 mg d-methylphenidate hydrochloride, respectively. Blood samples for PK were collected pre-dose and at 0.5, 1, 2, 4, 8, 10, 12, 13, 24, 36, and 48 hours post-dose. Adverse events were continuously recorded, and safety assessments were conducted throughout the study.

Results: Mean ages and weights were 7.0 years and 29.3 kg in Cohort 1, 10.1 years and 39.8 kg in Cohort 2, and 13.9 years and 65.4 kg in Cohort 3. Dose-normalized (to the 12/56 mg dose) peak and overall exposure to d-methylphenidate was highest in Cohort 1 (Cₘₐₓ=34.4 ng/mL, AUC₀₋₂₄=362.0 h*ng/mL), followed by Cohort 2 (Cₘₐₓ=25.9 ng/mL, AUC₀₋₂₄=294.1 h*ng/mL), and lowest in Cohort 3 (Cₘₐₓ=17.8 ng/mL and 14.0 ng/mL, for the low and high doses, respectively; AUC₀₋₂₄=195.0 ng/mL and 171.1 h*ng/mL, respectively). As shown in Table 7 below, when scaled by body weight, mean dose-normalized Cₘₐₓ (range across the 3 cohorts: 25.0-25.3 ng/mL/(mg/kg)) and AUC₀₋₂₄ (range across cohorts: 259.4-291.8 (h*ng/mL/(mg/kg)) values were similar across cohorts. Median time to peak d-methylphenidate exposure (Tₘₐₓ) was 4 hours in all cohorts. Clearance (CL/F) values were lower in Cohorts 1 and 2 (96.85 and 97.44 L/h, respectively) than Cohort 3 (170.3 L/h for low dose and 172.3 L/h for high dose), although when adjusted for weight differences, clearance values were similar. A nonlinear regression model evaluating allometric scaling indicated a moderate correlation (R²=0.628) between d-methylphenidate clearance (CL/F) and body weight and a weak correlation (R²=0.200) between d-methylphenidate volume of distribution (V_{Z}/F) and body weight. The geometric means and 95% CIs were within the target range of 60% to 140% for d-methylphenidate CL/F and for V_{Z}/F in all three cohorts. The geometric means and 95% CIs were also within the target range of 60% to 140% for d-methylphenidate CL/F for both dose groups of Cohort 3 and for the low-dose group (6/28 mg d-methylphenidate hydrochloride/serdexmethylphenidate chloride) of Cohort 3. (See Table 8) A total of 5 subjects reported AEs, none of which were serious or led to discontinuation.

TABLE 7
Mean PK parameters of d-methylphenidate following oral
administration of serdexmethylphenidate
chloride and d-methylphenidate
hydrochloride in children and adolescents:

| | | | AUC₀₋ | | | | | |
|---|---|---|---|---|---|---|---|---|
| | | Cₘₐₓ | _{24 hr} | AUCₗₐₛₜ | AUC_{inf} | | | |
| | | (ng/ | (h*ng/ | (h*ng/ | (h*ng/ | | | |
| | | mL/ | mL/ | mL/ | mL/ | CL/F | Vz/F | |
| Co- | Treat- | (mg/ | (mg/ | (mg/ | (mg/ | (L/h/ | (L/ | Tₘₐₓ |
| hort^{a} | ment^{b} | kg)) | kg)) | kg)) | kg)) | kg) | kg) | (h)^{c} |
| Cohort | A | 25 | 259.4 | 316.2 | 328.3 | 3.36 | 57.48 | 3 |
| 1 | | | | | | | | (4) |
| Cohort | B | 25.3 | 282.6 | 375.5 | 443.5 | 2.449 | 66.02 | 4.6 |
| 2 | | | | | | | | (4) |
| Cohort | Comb- | 25.3 | 291.85 | 357.9 | 386.3 | 2.611 | 39.72 | 3.6 |
| 3 | ined | | | | | | | (4) |
| | A and B | | | | | | | |
| Cohort | A | 27.8 | 306.9 | 364.9 | 393 | 2.564 | 37.6 | 3.2 |
| 3 | | | | | | | | (4) |
| Cohort | B | 22.8 | 276.8 | 350.9 | 379.6 | 2.658 | 41.84 | 4 |
| 3 | | | | | | | | (4) |

^{a}Cohort 1 = age 6-8 years, Cohort 2 = age 9-12 years, Cohort 3 = 13-17 years
^{b}A = 6/28 mg d-methylphenidate hydrochloride/serdexmethylphenidate chloride,
B = 12/56 mg d-methylphenidate hydrochloride/serdexmethylphenidate clloride
^{C}median Tₘₐₓ shown in parentheses

TABLE 8
Geometric means and 95% confidence intervals of
d-methylphenidate clearance (CL/F) and volume
of distribution (Vz/F) by cohort
following oral administration of serdexmethylphenidate
chloride and d-methylphenidate hydrochloride
in children and adolescents:

| | | | | | 95% | 95% |
|---|---|---|---|---|---|---|
| | | | | | CI^{c} | CI^{c} |
| | | | | Geometric | Lower | Upper |
| Cohort^{a} | Treatment^{b} | Parameter | n | Mean | (%) | (%) |
| Cohort 1 | A | CL/F (L/h/kg) | 9 | 3.179 | 77.55 | 128.94 |
| | | V_{z}/F (L/kg) | 9 | 56.07 | 83.84 | 119.27 |
| Cohort 2 | B | CL/F (L/h/kg) | 10 | 2.349 | 80.43 | 124.33 |
| | | V_{z}/F (L/kg) | 10 | 59.97 | 71.98 | 138.93 |
| Cohort 3 | Combined | CL/F (L/h/kg) | 10 | 2.600 | 93.19 | 107.31 |
| | A and B | V_{z}/F (L/kg) | 10 | 38.45 | 82.61 | 121.06 |
| Cohort 3 | A | CL/F (L/h/kg) | 5 | 2.554 | 88.42 | 113.10 |
| | | V_{z}/F (L/kg) | 5 | 36.91 | 76.79 | 130.22 |
| Cohort 3 | B | CL/F (L/h/kg) | 5 | 2.647 | 87.64 | 114.10 |
| | | V_{z}/F (L/kg) | 5 | 40.05 | 66.10 | 151.28 |

^{a}Cohort 1 = age 6-8 years, Cohort 2 = age 9-12 years, Cohort 3 = 13-17 years
^{b}A =6/28 mg d-methylphenidate hydrochloride/serdexmethylphenidate chloride,
B = 12/56 mg d-methylphenidate hydrochloride/serdexmethylphenidate chloride
^{v}95% Confidence Intervals (CI) expressed as a percentage of the geometric mean

This study showed that systemic dose-normalized exposure to d-methylphenidate following oral administration of d-methylphenidate and serdexmethylphenidate was higher in younger children, which appears to be due to lower clearance in younger children which is, in turn, primarily related to intrinsic body weight differences across the age spectrum examined in this study. The study results indicate that the combination of d-methylphenidate and serdexmethylphenidate produces predictable, age-dependent exposure to d-methylphenidate in pediatric subjects.

### Example 2: Intravenous Abuse Potential and Pharmacokinetic Study

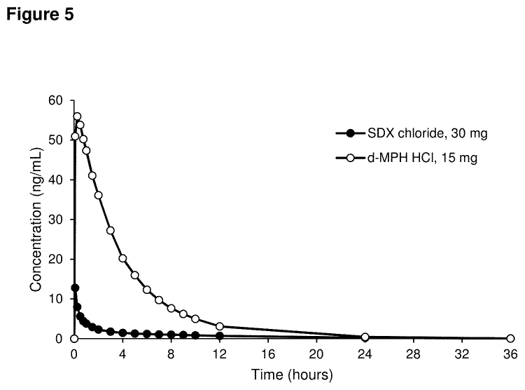

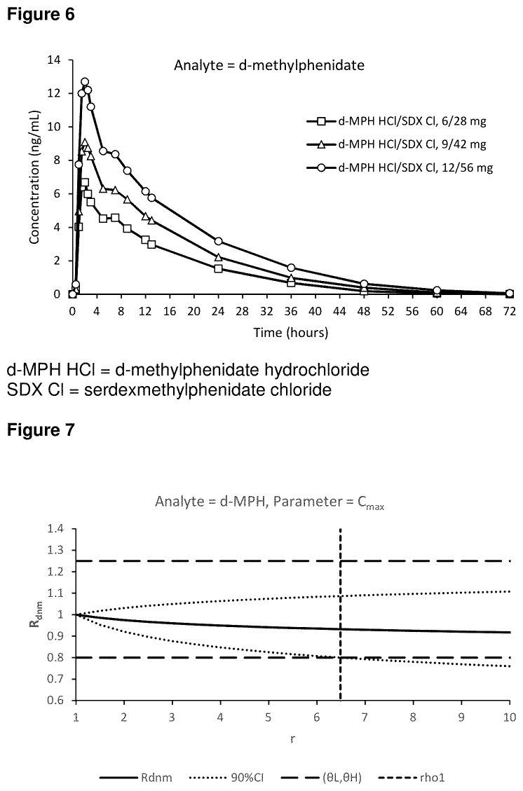

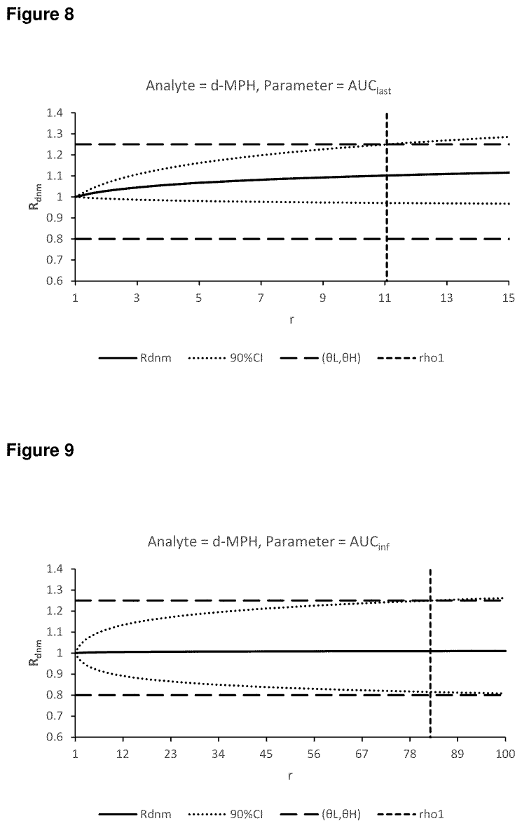

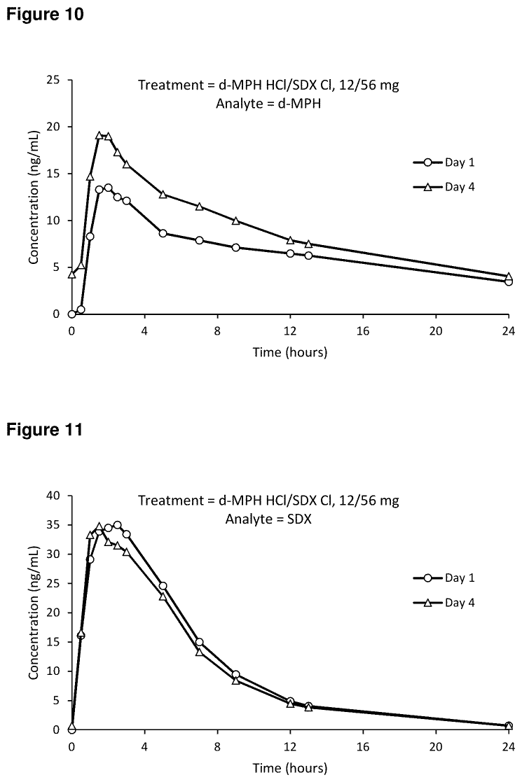

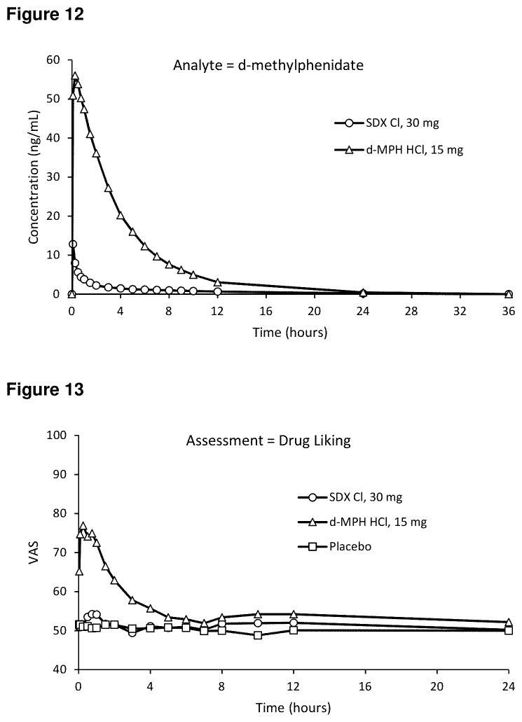

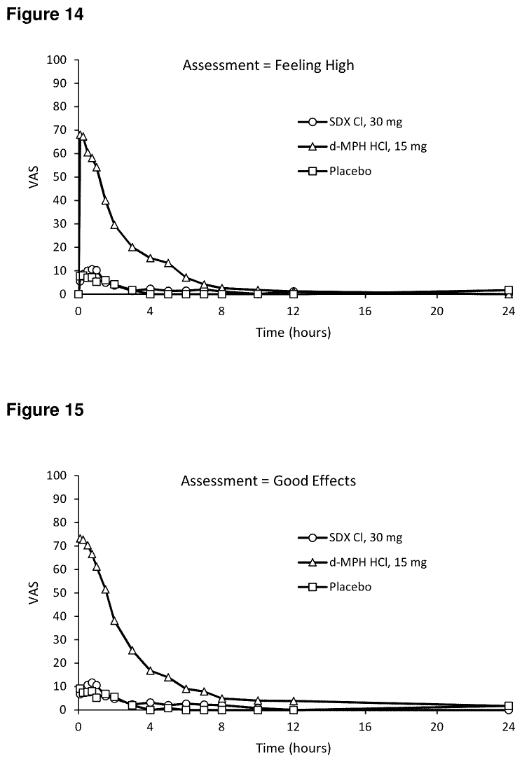

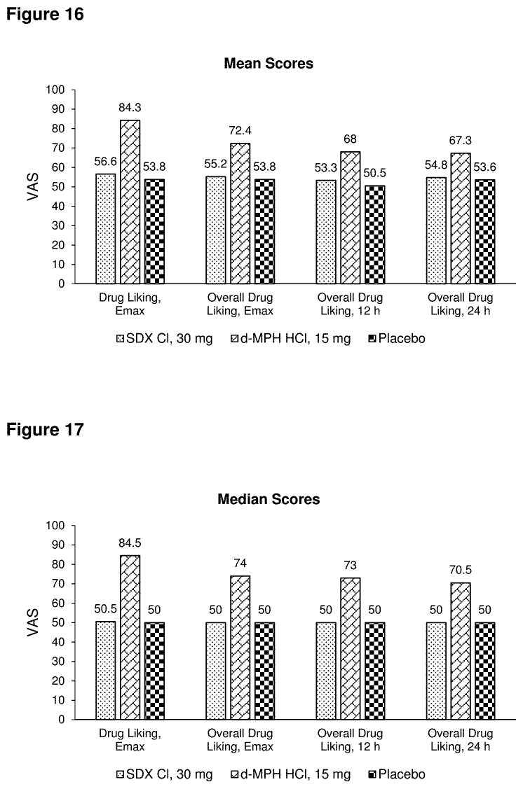

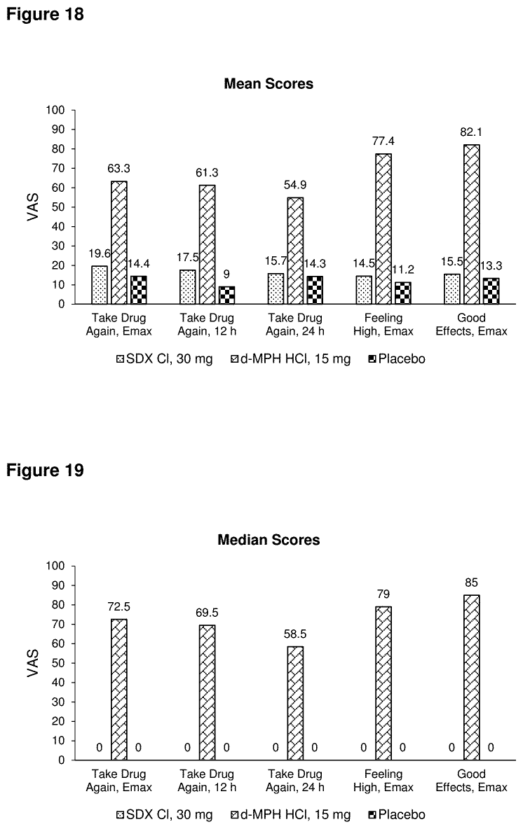

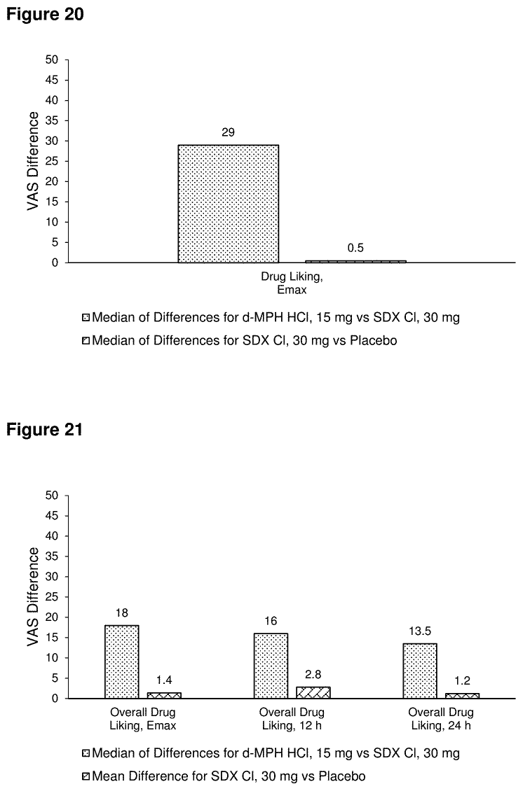

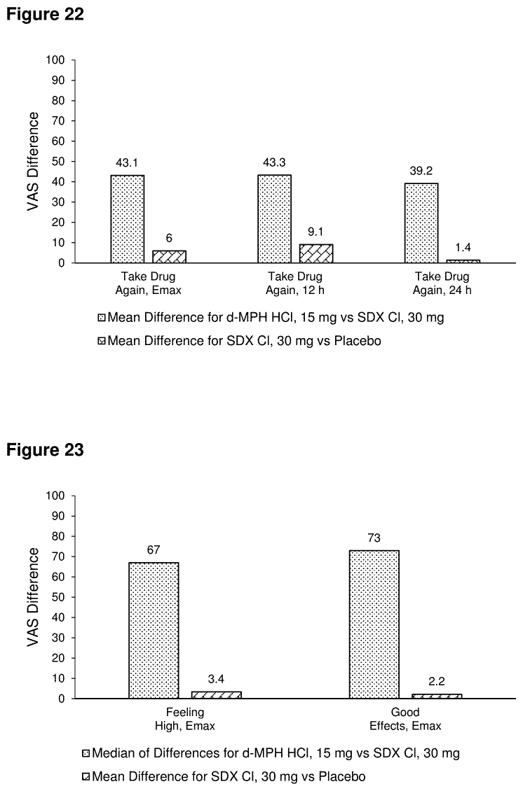

A study was conducted in humans to assess the intravenous abuse potential of serdexmethylphenidate relative to unconjugated d-methylphenidate and placebo in recreational stimulant abusers. This was a Phase 1, randomized, double-blind study in which serdexmethylphenidate and d-methylphenidate were administered intravenously in recreational stimulant users with a history of non-oral abuse. In Part A of the study, subjects (Cohort 1) participated in a dose-escalation phase to determine the optimal dose of intravenous d-methylphenidate based on tolerability and abuse-related pharmacodynamic assessments. In Part B, subjects (Cohort 2) who were able to discriminate the optimal dose of d-methylphenidate hydrochloride (15 mg) from placebo entered the Treatment Phase, consisting of a 3-treatment, 3-period, crossover design in which subjects received intravenous administration of serdexmethylphenidate chloride (30 mg), d-methylphenidate hydrochloride (15 mg), and placebo. The doses of serdexmethylphenidate and d-methylphenidate are equivalent with respect to molar amount of d-methylphenidate. FDA-recommended abuse potential measures and blood samples were collected at different times after dosing. Safety assessments were performed throughout the study. Assessment results are shown in the Tables below. FIG. 5 illustrates the d-methylphenidate time-plasma concentration profile.

Results: Thirty (n=30) subjects completed the study. Following intravenous administration of serdexmethylphenidate conjugate, d-methylphenidate exposure was dramatically reduced relative to administered d-methylphenidate. There was very little conversion of serdexmethylphenidate to d-methylphenidate following intravenous administration of serdexmethylphenidate compared to intravenous administration of unconjugated d-methylphenidate. Peak (Cₘₐₓ) and overall exposure (AUCₗₐₛₜ and AUC_{inf}) of d-methylphenidate for serdexmethylphenidate were approximately 20% and 10-15% of the respective PK parameter for 5unconjugated d-methylphenidate. Consistent with these observations, mean at the moment Drug Liking scores (assessed on a 100-point bipolar visual analog scale [VAS]) remained within the placebo range throughout the entire measurement interval. Statistical analyses of the primary endpoint, Drug Liking Eₘₐₓ, indicated that Eₘₐₓ was significantly higher for unconjugated d-methylphenidate (84.3) vs. placebo (53.8) (demonstrating study validity), significantly lower for serdexmethylphenidate (56.6) vs. d-methylphenidate (84.3), and not significantly different for serdexmethylphenidate (56.6) vs. placebo (53.8). The same general pattern of differences was observed for secondary endpoints including Take Drug Again, Feeling High, and Good Effects. Typical stimulant-related Adverse Events, such as euphoric mood, hypervigilance, and increased heart rate were more common during d-methylphenidate treatment vs serdexmethylphenidate.

TABLE 9
Mean PK parameters of d-methylphenidate released from
serdexmethylphenidate chloride
and d-methylphenidate hydrochloride:

| | | | | AUCₗₐₛₜ | AUC_{inf} | |
|---|---|---|---|---|---|---|
| | | Cₘₐₓ | Tₘₐₓ | (ng*h/ | (ng*h/ | T_{1/2} |
| Treatment | Statistic | (ng/mL) | (h) | mL) | mL) | (h) |
| A | N | 30 | 30 | 30 | 29 | 29 |
| | Mean | 12.9 | 0.0989 | 25.7 | 31.0 | 8.23 |
| | SD | 4.68 | 0.0446 | 10.6 | 11.5 | 3.43 |
| B | N | 30 | 30 | 30 | 30 | 30 |
| | Mean | 60.1 | 0.375 | 241 | 245 | 3.89 |
| | SD | 15.2 | 0.301 | 62.4 | 62.7 | 0.837 |

N = analysis sample size
SD = standard deviation
A = Serdexmethylphenidate chloride, 30 mg
B = d-methylphenidate hydrochloride, 15 mg

TABLE 10
Statistical analysis of PK parameters of d-methylphenidate for
the comparison of serdexmethylphenidate chloride vs
d-methylphenidate hydrochloride:

| | Intra- | | | GLSM | 90% Confidence | |
|---|---|---|---|---|---|---|
| | Subject | | | Ratio | Interval (%) | |
| | CV | GLSM | | A/B | Lower | Upper |
| Parameter | (%) | A | B | (%) | Bound | Bound |
| AUCₗₐₛₜ | 21.0 | 23.8 | 233.7 | 10.2 | 9.3 | 11.2 |
| AUC_{inf} | 17.8 | 28.9 | 237.6 | 12.2 | 11.3 | 13.2 |
| Cₘₐₓ | 28.9 | 12.1 | 58.3 | 20.8 | 18.3 | 23.5 |

CV = coefficient of variation
GLSM = geometric least squares mean
A = Serdexmethylphenidate chloride, 30 mg
B = d-methylphenidate hydrochloride, 15 mg

TABLE 11

| | Mean Eₘₐₓ Scores | | | | |
|---|---|---|---|---|---|
| | Drug | Take Drug | Overall Drug | Feeling | Good |
| Treatment | Liking^{a} | Again^{b} | Liking^{a} | High^{b} | Effects^{b} |
| A | 56.6 | 19.6 | 55.2 | 14.5 | 15.5 |
| B | 84.3 | 63.3 | 72.4 | 77.4 | 82.1 |
| C | 53.8 | 14.4 | 53.8 | 11.2 | 13.3 |

^{a}assessed on a bipolar VAS
^{b}assessed on a unipolar VAS
A = Serdexmethylphenidate chloride, 30 mg
B = d-methylphenidate hydrochloride, 15 mg
C = Placebo

TABLE 12

| | Median Eₘₐₓ Scores | | | | |
|---|---|---|---|---|---|
| | Drug | Take Drug | Overall Drug | Feeling | Good |
| Treatment | Liking^{a} | Again^{b} | Liking^{a} | High^{b} | Effects^{b} |
| A | 50.5 | 0.0 | 50.0 | 0.0 | 0.0 |
| B | 84.5 | 72.5 | 74.0 | 79.0 | 85.0 |
| C | 50.0 | 0.0 | 50.0 | 0.0 | 0.0 |

^{a}assessed on a bipolar VAS
^{b}assessed on a unipolar VAS
A = Serdexmethylphenidate chloride, 30 mg
B = d-methylphenidate hydrochloride, 15 mg
C = Placebo

TABLE 13

| | Mean Take Drug Again^{a} | | Mean Overall Drug Liking^{a} | |
|---|---|---|---|---|
| Treatment | 12 hours | 24 hours | 12 hours | 24 hours |
| A | 17.5 | 15.7 | 53.3 | 54.8 |
| B | 61.3 | 54.9 | 68.0 | 67.3 |
| C | 9.0 | 14.3 | 50.5 | 53.6 |

^{a}assessed on a unipolar VAS
^{b} assessed on a bipolar VAS
A = Serdexmethylphenidate chloride, 30 mg
B = d-methylphenidate hydrochloride, 15 mg

TABLE 14

| | Median Take Drug Again^{a} | | Median Overall Drug Liking^{a} | |
|---|---|---|---|---|
| Treatment | 12 hours | 24 hours | 12 hours | 24 hours |
| A | 0.0 | 0.0 | 50.0 | 50.0 |
| B | 69.5 | 58.5 | 73.0 | 70.5 |
| C | 0.0 | 0.0 | 50.0 | 50.0 |

^{a}assessed on a unipolar VAS
^{b} assessed on a bipolar VAS
A = Serdexmethylphenidate chloride, 30 mg
B = d-methylphenidate hydrochloride, 15 mg
C = Placebo

TABLE 15

| | Median Difference |
|---|---|
| | Drug Liking^{a} |
| Treatment | Eₘₐₓ |
| B vs A | 29.0 |
| A vs C | 0.5 |

^{a}assessed on a bipolar VAS
A = Serdexmethylphenidate chloride, 30 mg
B = d-methylphenidate hydrochloride, 15 mg
C = Placebo

TABLE 16

| | Mean Difference | | |
|---|---|---|---|
| | Take Drug Again^{a} | | |
| Treatment | 12 hours | 24 hours | Eₘₐₓ |
| B vs A | 43.3 | 39.2 | 43.1 |
| A vs C | 9.1 | 1.4 | 6.0 |

^{a}assessed on a unipolar VAS
A = Serdexmethylphenidate chloride, 30 mg
B = d-methylphenidate hydrochloride, 15 mg
C = Placebo

TABLE 17

| | Overall Drug Liking^{a} | | | |
|---|---|---|---|---|
| Treatment | Difference | 12 hours | 24 hours | Eₘₐₓ |
| B vs A | Median difference | 16.0 | 13.5 | 18.0 |
| A vs C | Mean difference | 2.8 | 1.2 | 1.4 |

^{a}assessed on a bipolar VAS
A = Serdexmethylphenidate chloride, 30 mg
B = d-methylphenidate hydrochloride, 15 mg
C = Placebo

TABLE 18

Feeling High^{a}

| Treatment | Difference | Eₘₐₓ |
|---|---|---|
| B vs A | Median difference | 67.0 |
| A vs C | Mean difference | 3.4 |

^{a}assessed on a unipolar VAS
A = Serdexmethylphenidate chloride, 30 mg
B = d-methylphenidate hydrochloride, 15 mg
C = Placebo

TABLE 19

Good Effects^{a}

| Treatment | Difference | Eₘₐₓ |
|---|---|---|
| B vs A | Median difference | 73.0 |
| A vs C | Mean difference | 2.2 |

^{a}assessed on a unipolar VAS
A = Serdexmethylphenidate chloride, 30 mg
B = d-methylphenidate hydrochloride, 15 mg
C = Placebo

TABLE 20
Adverse Events

| | Treatment at Onset of Adverse Event | | |
|---|---|---|---|
| | SDX Cl, 30 mg | d-MPH HCl, 15 mg | Placebo |
| | (N = 31) | (N = 30) | (N = 31) |
| TEAE^{a} | n (%)^{b} | n (%)^{b} | n (%)^{b} |

Cardiac disorders

| | | | |
|---|---|---|---|
| Palpitations | 0 (0.0) | 2 (6.7) | 0 (0.0) |
| Sinus tachycardia | 0 (0.0) | 4 (13.3) | 0 (0.0) |
| Tachycardia | 0 (0.0) | 4 (13.3) | 0 (0.0) |

Gastrointestinal disorders

| | | | |
|---|---|---|---|
| Dry mouth | 0 (0.0) | 6 (20.0) | 0 (0.0) |
| Nausea | 0 (0.0) | 3 (10.0) | 0 (0.0) |

General disorders and administration site conditions

| | | | |
|---|---|---|---|
| Energy increased | 2 (6.5) | 1 (3.3) | 1 (3.2) |
| Feeling abnormal | 1 (3.2) | 2 (6.7) | 0 (0.0) |
| Feeling hot | 2 (6.5) | 6 (20.0) | 2 (6.5) |
| Feeling jittery | 0 (0.0) | 2 (6.7) | 0 (0.0) |
| Feeling of relaxation | 0 (0.0) | 1 (3.3) | 2 (6.5) |

Investigations

| | | | |
|---|---|---|---|
| Heart rate increased | 0 (0.0) | 5 (16.7) | 0 (0.0) |

Nervous system disorders

| | | | |
|---|---|---|---|
| Headache | 1 (3.2) | 1 (3.3) | 2 (6.5) |
| Paraesthesia (tingling) | 0 (0.0) | 2 (6.7) | 1 (3.2) |
| Somnolence | 1 (3.2) | 4 (13.3) | 0 (0.0) |

Psychiatric disorders

| | | | |
|---|---|---|---|
| Change in sustained | 0 (0.0) | 2 (6.7) | 0 (0.0) |
| attention | | | |
| Euphoric mood | 4 (12.9) | 17 (56.7) | 2 (6.5) |
| Hypervigilance | 4 (12.9) | 10 (33.3) | 2 (6.5) |

Skin and subcutaneous tissue disorders

| | | | |
|---|---|---|---|
| Hyperhidrosis (sweaty | 1 (3.2) | 4 (13.3) | 0 (0.0) |
| hands) | | | |

^{a}a Treatment-Emergent Adverse Event (TEAE) is an adverse event which starts or worsens on or after treatment with study drug in the treatment phase.
^{b}n = number of subjects in which the adverse event occurred; percentages are calculated as ratio of number of subjects reporting an adverse event and total number of subjects receiving the respective treatment

This study demonstrates that, in subjects with a history of non-oral abuse of stimulants, intravenous serdexmethylphenidate produced effects that were statistically similar to intravenous placebo on multiple abuse-related endpoints.

### Example 3: ADHD Efficacy Study

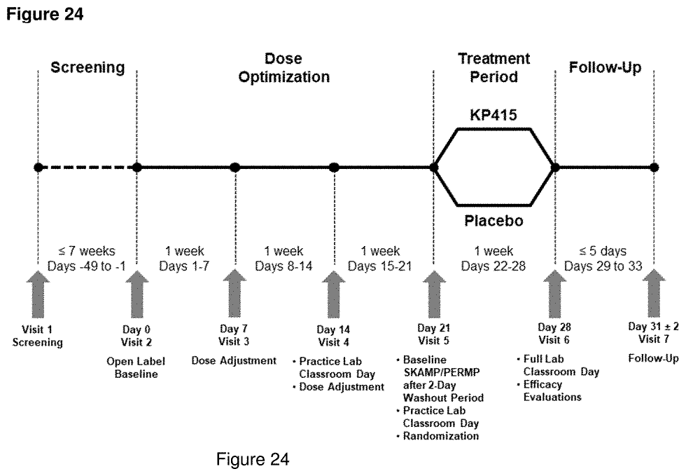

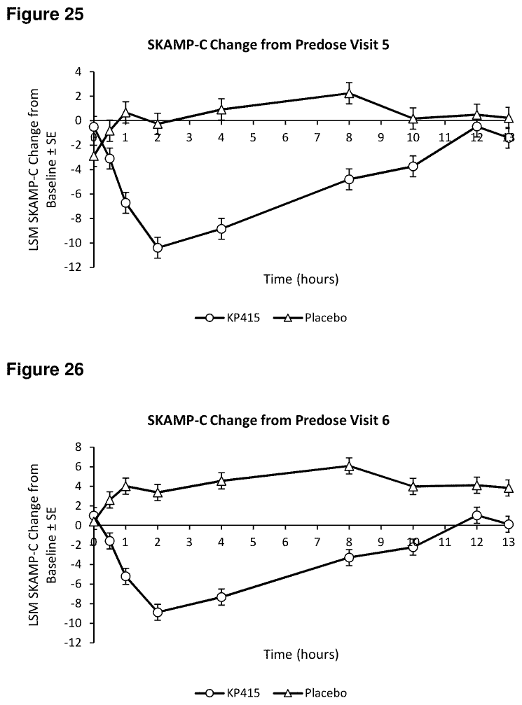

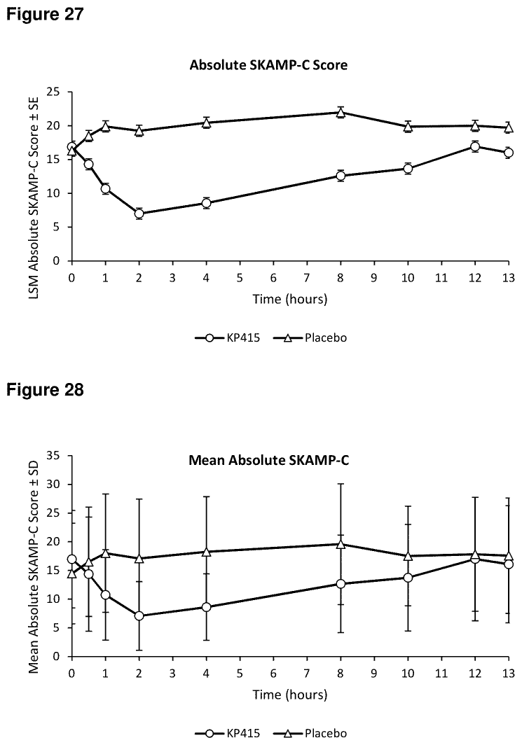

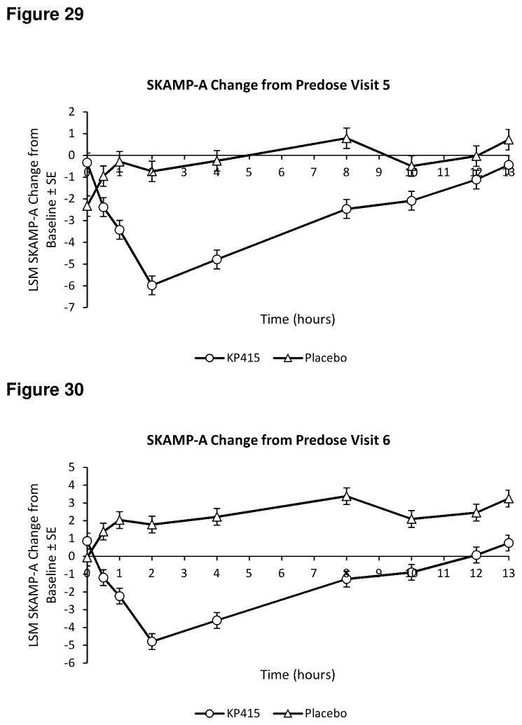

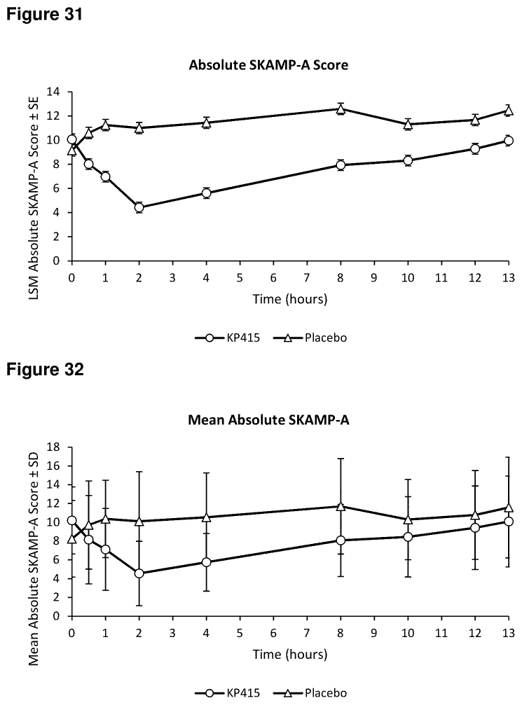

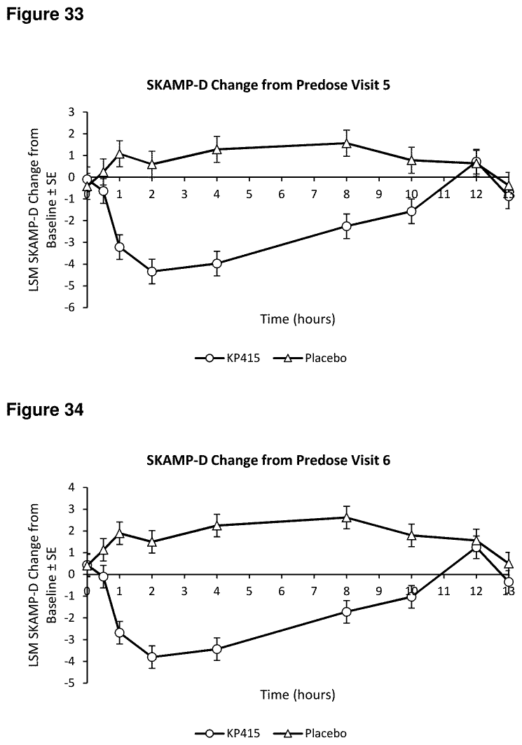

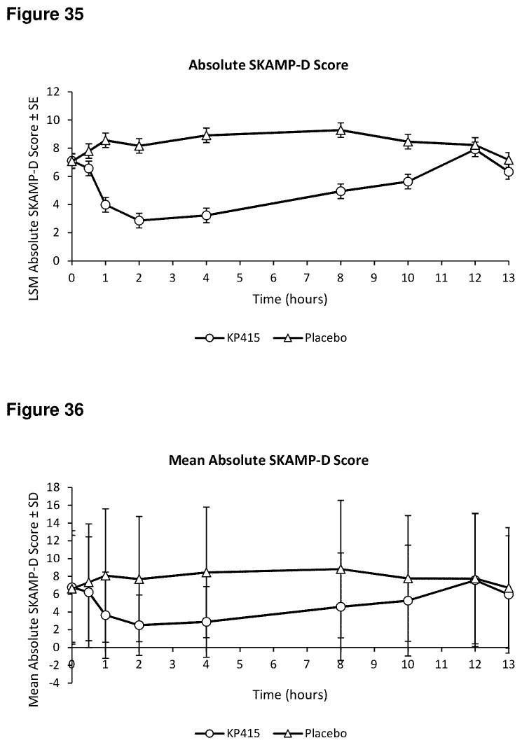

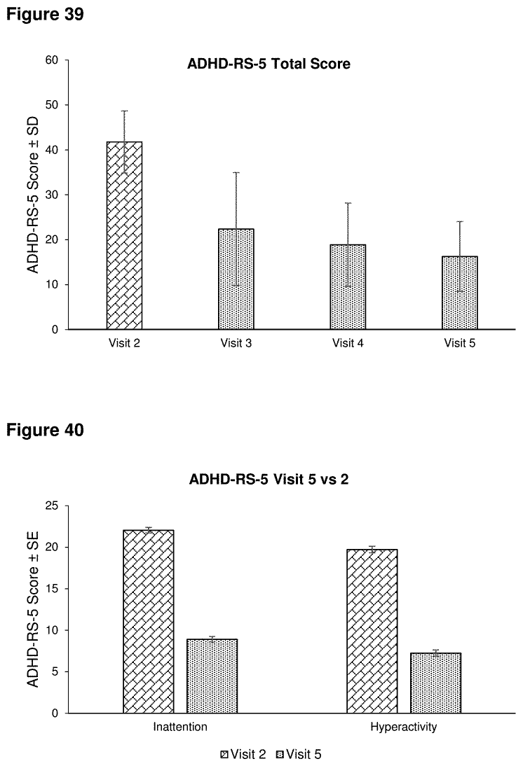

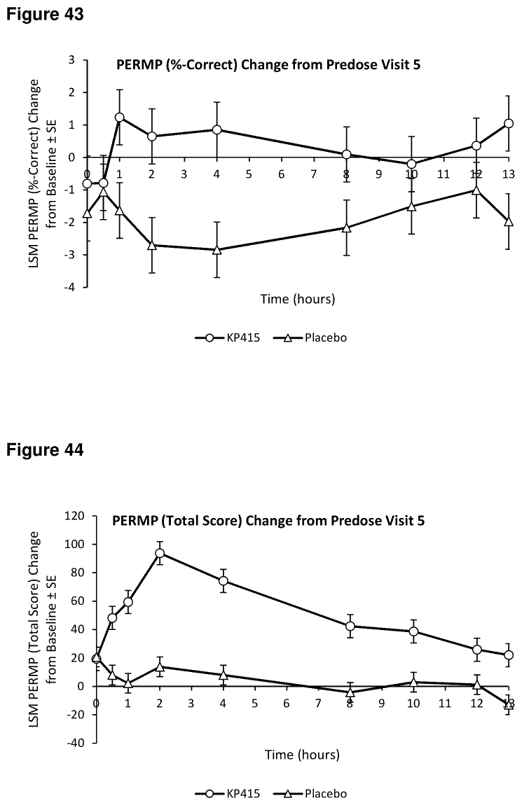

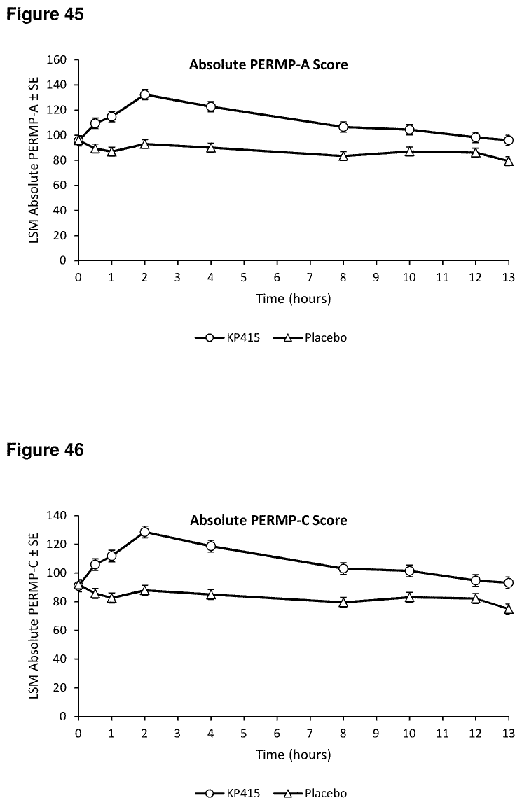

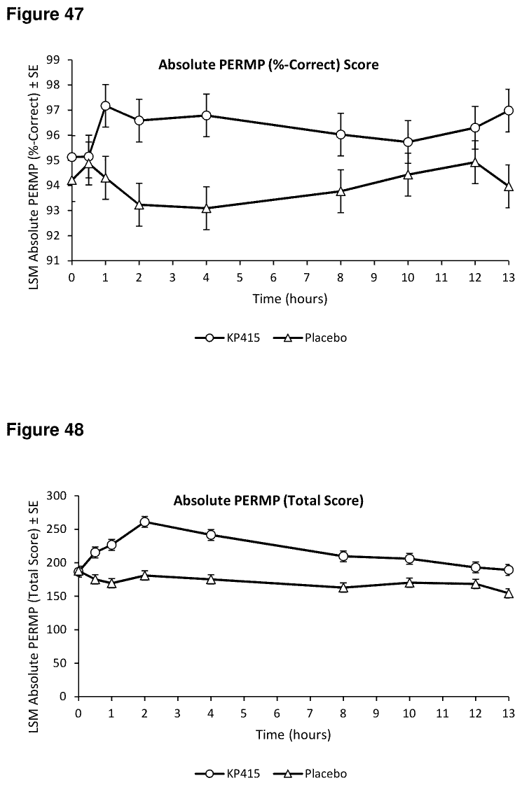

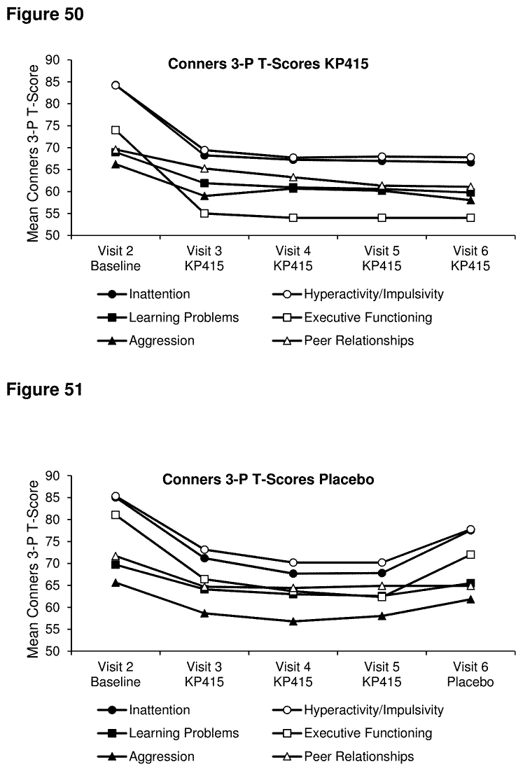

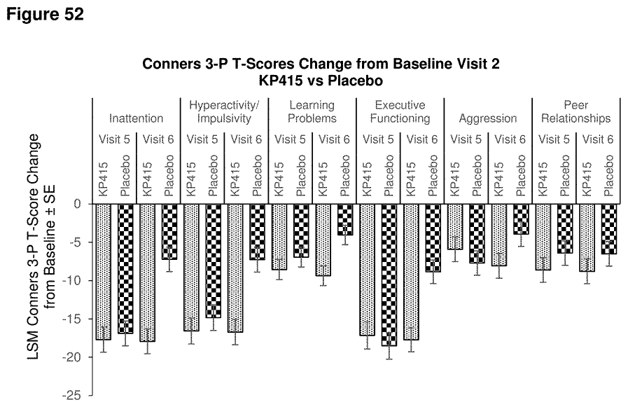

A study was conducted to assess the efficacy of serdexmethylphenidate relative to placebo in children with ADHD ages 6 to 12 years. This was a double-blind, placebo-controlled, randomized, parallel, analog classroom study in which serdexmethylphenidate and d-methylphenidate were orally administered to children ages 6 to 12 years with diagnosed ADHD. The study was conducted at 5 study sites, 2-3 cohorts each, with 5 to 18 subjects per cohort per site. FIG. 24 shows the design schematic for the ADHD efficacy study. After an eligibility screening period of up to 7 weeks (screening phase—Visit 1), subjects entered a 3-week open-label dose optimization phase (Visit 2) in which subjects were administered once-per-day dosing of d-methylphenidate hydrochloride/serdexmethylphenidate chloride, at a 30%/70% fixed molar dose ratio. Each subject was administered a daily dose of 9/42 mg d-methylphenidate hydrochloride/serdexmethylphenidate chloride, equimolar to 30 mg d-methylphenidate hydrochloride, during the first week. After week 1 (Visit 3) and after week 2 (Visit 4) of the optimization phase, individual doses were adjusted up to 12/56 mg, equimolar to 40 mg d-methylphenidate hydrochloride, or down to 6/28 mg, equimolar to 20 mg d-methylphenidate hydrochloride, based on tolerability and individual dose response. WREMB-R, ADHD-RS-5, and Conners 3-P assessments were also performed during the optimization phase. The 3-week dose optimization phase ended with a 2-day drug wash out period. On Visit 5 (day 21), baseline SKAMP-C (including SKAMP-A and SKAMP-D) scores, and PERMP (including PERMP-A and PERMP-C) scores were collected at pre-dose. Subjects were then given their last open-label dose and were randomly assigned to either placebo or their optimized dose of d-methylphenidate/serdexmethylphenidate. Doses were administered once daily in the morning for 1 week. On Visit 6 (day 28), SKAMP-C and PERMP scores were collected at pre-dose, and then post-dose efficacy assessments were taken at 0.5, 1, 2, 4, 8, 10, 12, and 13 hours. SKAMP-C scores were analyzed using a mixed-effect model repeated measure (MMRM) approach with time, treatment, interaction of time and treatment as fixed effects, and subject as random effect. Clinical site can be added as optional fixed effect, and baseline can be added as optional covariate or fixed effect. The study results are shown in FIGS. 25-52.

Results: One hundred fifty (n=150) subjects completed the study. Using the Visit 5 pre-dose SKAMP-C scores as a baseline, there was a significant difference in SKAMP-C scores between the d-methylphenidate hydrochloride/serdexmethylphenidate chloride test drug and placebo for the post-dose time periods of 1 hour through 10 hours. Using the Visit 6 pre-dose SKAMP-C scores as a baseline, there was a significant difference in SKAMP-C scores between the d-methylphenidate hydrochloride/serdexmethylphenidate chloride test drug and placebo for the post-dose time periods of 0.5 hours through 13 hours. Differences in SKAMP-C scores are presented in Table 21A and are graphically shown in FIGS. 25-36.

TABLE 21A

| | SKAMP-C Change from Baseline | | | |
|---|---|---|---|---|
| | Baseline = predose | | Baseline = predose | |
| | Visit 5^{a} | | Visit 6^{a} | |
| | Difference in | | Difference in | |
| | LS mean (SE) | | LS mean (SE) | |
| | Active^{b} − | | Active^{b} − | |
| Time | Placebo | p-Value | Placebo | p-Value |
| Predose | 2.37 (1.18) | 0.044 | 0.599 (1.14) | 0.600 |
| 0.5 hours postdose | −2.28 (1.18) | 0.053 | −4.19 (1.14) | <0.001 |
| 1 hours postdose | −7.40 (1.18) | <0.001 | −9.22 (1.14) | <0.001 |
| 2 hours postdose | −10.14 (1.18) | <0.001 | −12.25 (1.14) | <0.001 |
| 4 hours postdose | −9.76 (1.18) | <0.001 | −11.88 (1.14) | <0.001 |
| 8 hours postdose | −7.05 (1.18) | <0.001 | −9.37 (1.14) | <0.001 |
| 10 hours postdose | −3.91 (1.18) | <0.001 | −6.20 (1.14) | <0.001 |
| 12 hours postdose | −0.96 (1.18) | 0.412 | −3.07 (1.14) | 0.007 |
| 13 hours postdose | −1.63 (1.18) | 0.167 | −3.71 (1.14) | 0.001 |

^{a}Statistical model includes predose Visit 5 or Visit 6 as covariate, respectively.
^{b}Active = d-methylphenidate hydrochloride/serdexmethylphenidate chloride

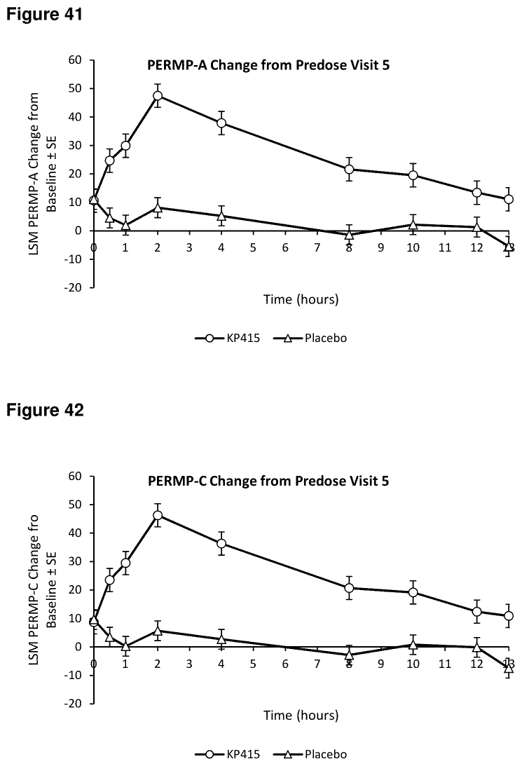

There was also a significant difference in PERMP scores between the d-methylphenidate hydrochloride/serdexmethylphenidate chloride test drug and placebo for the post-dose time periods of 0.5 hours through 13 hours. Differences in PERMP scores are shown in FIGS. 41 to 48.

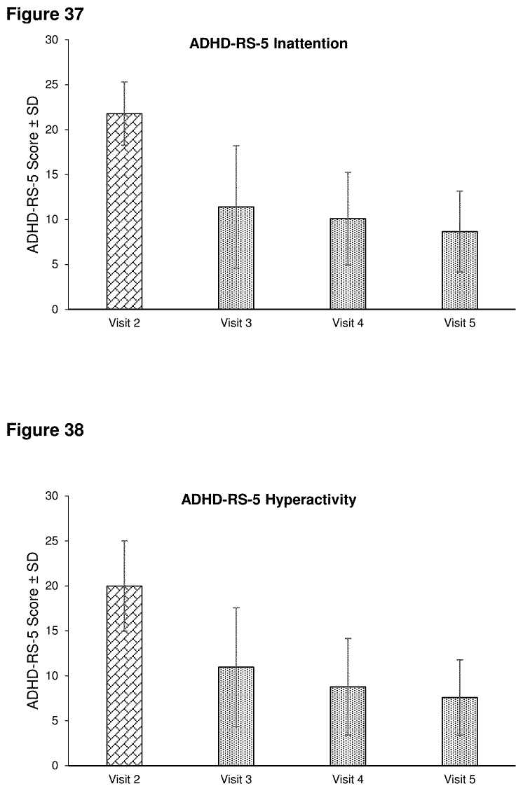

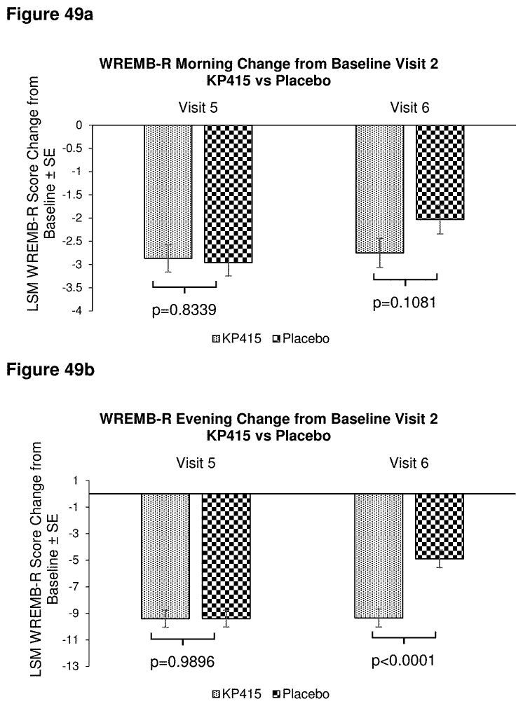

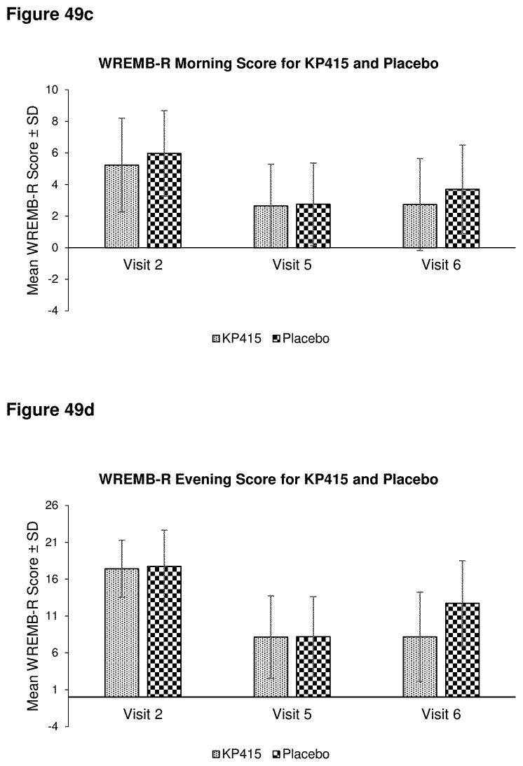

FIGS. 37 to 40 show ADHD-RS-5 test scores at visit 5 compared to visit 2. FIGS. 49 to 52 show the results from the WREMB-R and Conners 3-P score assessments.

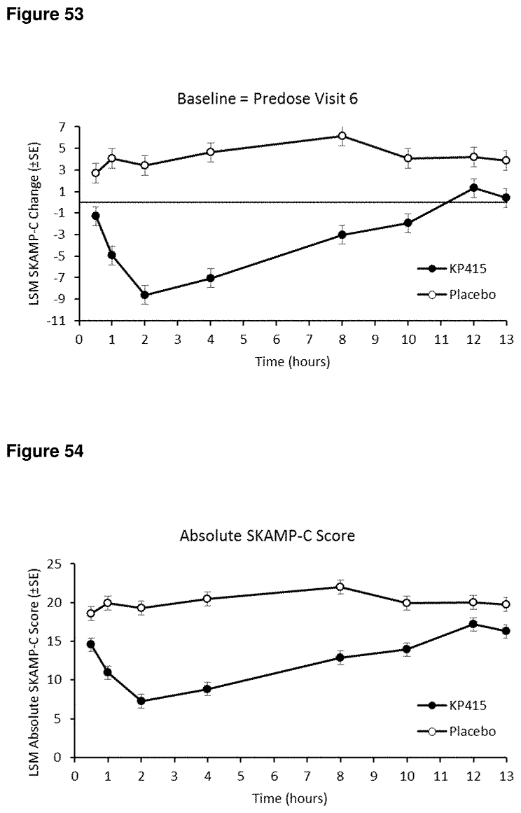

In an alternative analysis, only SKAMP-C score changes from predose Visit 6 at postdose time points were compared between d-methylphenidate hydrochloride/serdexmethylphenidate chloride and placebo. The resulting differences in SKAMP-C scores are shown in Table 21B and the plot of change in SKAMP-C scores from predose Visit 6 vs time are presented in FIG. 53. Using the Visit 6 pre-dose SKAMP-C score changes at postdose time points, there was a significant difference in SKAMP-C scores between the d-methylphenidate hydrochloride/serdexmethylphenidate chloride test drug and placebo for the postdose time periods of 0.5 hours through 13 hours.

TABLE 21B

| | Change in SKAMP-C from Predose Visit 6^{a} | | | | | |
|---|---|---|---|---|---|---|
| | | | Difference | 95% | | |
| | | | in LS mean | Confidence | | |
| Time | | | (SE) | Interval | | |
| Point | LS Mean (SE) | | Active^{c} − | Active^{c} − | | |
| Visit 6^{b} | Active^{c} | Placebo | Placebo | Placebo | | p-Value |
| 0.5 hours | −1.30 | 2.67 | −3.97 | −6.37 | −1.57 | 0.0012 |
| postdose | (0.88) | (0.91) | (1.22) | | | |
| 1 hours | −4.92 | 4.08 | −9.00 | −11.40 | −6.60 | <0.0001 |
| postdose | (0.88) | (0.91) | (1.22) | | | |
| 2 hours | −8.60 | 3.43 | −12.03 | −14.43 | −9.63 | <0.0001 |
| postdose | (0.88) | (0.91) | (1.22) | | | |
| 4 hours | −7.04 | 4.62 | −11.66 | −14.06 | −9.26 | <0.0001 |
| postdose | (0.88) | (0.91) | (1.22) | | | |
| 8 hours | −3.00 | 6.15 | −9.15 | −11.55 | −6.75 | <0.0001 |
| postdose | (0.88) | (0.91) | (1.22) | | | |
| 10 hours | −1.93 | 4.05 | −5.99 | −8.39 | −3.59 | <0.0001 |
| postdose | (0.88) | (0.91) | (1.22) | | | |
| 12 hours | 1.32 | 4.17 | −2.85 | −5.25 | −0.45 | 0.0200 |
| postdose | (0.88) | (0.91) | (1.22) | | | |
| 13 hours | 0.40 | 3.90 | −3.49 | −5.89 | −1.09 | 0.0044 |
| postdose | (0.88) | (0.91) | (1.22) | | | |
| Mean | −3.13 | 4.13 | −7.27 | −9.00 | −5.53 | <0.0001 |
| difference | (0.61) | (0.71) | (0.88) | | | |
| in change | | | | | | |
| from | | | | | | |
| predose | | | | | | |
| Visit 6 | | | | | | |
| across all | | | | | | |
| postdose | | | | | | |
| time points | | | | | | |

^{a}Statistical model includes predose Visit 6 as covariate
^{b}Statistical model includes only postdose time points
^{c}Active = d-methylphenidate hydrochloride/serdexmethylphenidate chloride

In yet a further analysis, absolute SKAMP-C scores at postdose time points of Visit 6 were compared between d-methylphenidate hydrochloride/serdexmethylphenidate chloride and placebo. The resulting differences in SKAMP-C scores are shown in Table 21C and the plot of absolute SKAMP-C scores vs time are presented in FIG. 54. Using the Visit6 absolute SKAMP-C scores at postdose time points, there was a significant difference in absolute SKAMP-C scores between the d-methylphenidate hydrochloride/serdexmethylphenidate chloride test drug and placebo for the post-dose time periods of 0.5 hours through 13 hours.

TABLE 21C

| | Absolute SKAMP-C Score^{a} | | | | | |
|---|---|---|---|---|---|---|
| | | | Difference | 95% | | |
| | | | in LS | Confidence | | |
| Time | | | mean (SE) | Interval | | |
| Point | LS Mean (SE) | | Active^{c} − | Active^{c} − | | |
| Visit 6^{b} | Active^{c} | Placebo | Placebo | Placebo | | p-Value |
| Predosed | 16.96 | 14.46 | — | — | — | — |
| | (0.99) | (1.02) | | | | |
| 0.5 hours | 14.57 | 18.54 | −3.97 | −6.37 | −1.57 | 0.0012 |
| postdose | (0.88) | (0.91) | (1.22) | | | |
| 1 hours | 10.95 | 19.95 | −9.00 | −11.40 | −6.60 | <0.0001 |
| postdose | (0.88) | (0.91) | (1.22) | | | |
| 2 hours | 7.28 | 19.31 | −12.03 | −14.43 | −9.63 | <0.0001 |
| postdose | (0.88) | (0.91) | (1.22) | | | |
| 4 hours | 8.83 | 20.49 | −11.66 | −14.06 | −9.26 | <0.0001 |
| postdose | (0.88) | (0.91) | (1.22) | | | |
| 8 hours | 12.87 | 22.02 | −9.15 | −11.55 | −6.75 | <0.0001 |
| postdose | (0.88) | (0.91) | (1.22) | | | |
| 10 hours | 13.94 | 19.93 | −5.99 | −8.39 | −3.59 | <0.0001 |
| postdose | (0.88) | (0.91) | (1.22) | | | |
| 12 hours | 17.20 | 20.04 | −2.85 | −5.25 | −0.45 | 0.0200 |
| postdose | (0.88) | (0.91) | (1.22) | | | |
| 13 hours | 16.28 | 19.77 | −3.49 | −5.89 | −1.09 | 0.0044 |
| postdose | (0.88) | (0.91) | (1.22) | | | |
| Mean | 12.74 | 20.01 | −7.27 | −9.00 | -5.53 | <0.0001 |
| difference | (0.615) | (0.71) | (0.88) | | | |
| in change | | | | | | |
| from | | | | | | |
| predose | | | | | | |
| Visit 6 | | | | | | |
| across all | | | | | | |
| postdose | | | | | | |
| time points | | | | | | |

^{a}Statistical model includes predose Visit 6 as covariate
^{b}Statistical model includes only postdose time points
^{c}Active = d-methylphenidate hydrochloride/serdexmethylphenidate chloride
^{d}Mean (SE) predose SKAMP-C shown

This study shows that, in some embodiments, d-methylphenidate hydrochloride /serdexmethylphenidate chloride provides a post-dose onset of action beginning as early as about 0.5 hours and a duration of efficacy of up to 13 hours post-dose, and provide a post-dose onset of action of about 0.5 hours and a duration of efficacy of about 13 hours post-dose, as shown by the SKAMP-C scores. In addition, the study results indicate overall efficacy of d-methylphenidate hydrochloride/serdexmethylphenidate chloride for treating ADHD, as shown by the ADHD-RS-5, WREMB-R and Conners 3-P assessments.

### Example 4: Oral Abuse Potential and Pharmacokinetic Study

A Phase 1, randomized, double-blind, single dose, active-and placebo-controlled, 5-treatment, 5-period, 10-sequence crossover study was conducted to evaluate the abuse potential and pharmacokinetics of 120 mg and 240 mg serdexmethylphenidate (SDX) chloride in capsules, extended-release d-methylphenidate hydrochloride (Focalin® XR 80 mg), 60 mg phentermine hydrochloride, and placebo, after oral administration in healthy, nondependent, recreational stimulant users. Focalin® XR is an extended-release formulation of dexmethylphenidate (d-methylphenidate) hydrochloride, available from Novartis AG, and uses the proprietary SODAS° (spheroidal Oral Drug Absorption System) technology. Focalin® XR is a Schedule II drug and is intended for oral administration once daily in the morning for the treatment of ADHD. The maximum clinical daily dosage is 40 mg. Phentermine is a structural analogue to amphetamine and has been approved as a therapy for obesity in the United States. Phentermine is a Schedule IV drug having a daily dose of 15 to 30 mg.

The study consisted of a Screening Period, an in-clinic Drug Discrimination Phase, an in-clinic Treatment Phase, and a Follow-Up Visit. Subjects who successfully completed the Screening Period returned to the clinic to complete the Drug Discrimination Phase. The Drug Discrimination Test was performed to ensure that subjects can differentiate between the effects of a single dose of Focalin® XR and placebo administered orally. The Drug Discrimination Phase had a double-blind, oral, single-dose, 2-treatment, 2-period, 2-sequence, randomized, crossover design. Subjects received single oral doses of the following treatments separated by a 48-hour washout period:
Treatment X: 80 mg Focalin® XR (2×40 mg capsules, overencapsulated).
Treatment Y: Placebo (2 matching placebo capsules).

All subjects were required to fast for at least 8 hours prior to each dose until approximately 4 hours after each dose. Abuse potential assessments were performed at different times after the administration of study drug. Subjects who successfully completed the Drug Discrimination Phase and who qualified for the Treatment Phase, returned to the research clinic to enter the Treatment Phase. After a washout period of at least 72 hours following the last dose in the Drug Discrimination Phase, subjects eligible to continue in the Treatment Phase were randomized into the Treatment Phase in a 1:1:1:1:1 ratio to receive 5 different treatments in a double-blind, crossover design, each separated by a minimum 96-hour washout period as follows:
Treatment A: 120 mg serdexmethylphenidate (SDX) chloride (2×60 mg capsules)
Treatment B: 240 mg serdexmethylphenidate (SDX) chloride (4×60 mg capsules)
Treatment C: 80 mg Focalin® XR (2×40 mg capsules)
Treatment D: 60 mg Phentermine hydrochloride (2×30 mg capsules)
Treatment E: Placebo (matching capsules)

Both Focalin® XR (Treatment C) and phentermine (Treatment D) were administered at twice the highest approved therapeutic dose as outlined in FDA guidance. SDX chloride at 240 mg (Treatment B) was equivalent in d-methylphenidate content (on a molar basis) to 120 mg or three times the highest approved dose of Focalin® XR. SDX chloride at 120 mg (Treatment B) was equivalent in d-methylphenidate content (on a molar basis) to 60 mg or one and a half times the highest approved dose of Focalin® XR.

Blinding of all treatments was accomplished by a double dummy approach using two types of matching placebo capsules, one for the test product (SDX chloride) and one for the overencapsulated positive control products (i.e., Focalin® XR and phentermine). The same number of capsules was administered for each treatment, and the capsules looked the same for each treatment.

On dosing days in the Treatment Phase, blood samples were collected for the measurement of the plasma concentrations of SDX, d-methylphenidate (d-MPH), 1-methylphenidate (1-MPH) and ritalinic acid at predose and at 0.5, 1, 1.5, 2, 3, 4, 5, 6, 7, 8, 9, 10, 11, 12, 14, 16, 24, 36, and 48 hours ±5 minutes after each dose of study drug.

Pharmacodynamic assessments included VAS assessments of Drug Liking, Good Effects, Bad Effects, Any Effects, Feeling High, Drowsiness/Alertness, Take Drug Again, and Overall Drug Liking. All VAS pharmacodynamic assessments except Take Drug Again and Overall Drug Liking were performed at 0.5, 1, 1.5, 2, 3, 4, 5, 6, 7, 8, 10, 12, 14, and 16 hours ±5 minutes postdose. In addition, predose assessments of Feeling High, and Drowsiness/Alertness were collected. The Take Drug Again and Overall Drug Liking VAS assessments were performed at 12 hours ±5 minutes postdose.

Pharmacodynamic endpoint VAS scales:
Bipolar VAS “At this Moment” Drug Liking (postdose assessments).
Unipolar VAS “At this Moment” Good Effects, Bad Effects (postdose assessments).
Unipolar Any Effects (postdose assessments).
Unipolar VAS “At this Moment” Feeling High (predose and postdose assessments).
Bipolar VAS Drowsiness/Alertness (predose and postdose assessments).
Bipolar VAS Take Drug Again at 12 and 24 hour postdose.
Bipolar VAS Overall Drug Liking at 12 and 24 hour postdose.

### Primary Pharmacodynamic Endpoint:

VAS Drug Liking Eₘₐₓ: Focalin® XR vs. placebo, SDX chloride vs. Focalin® XR, and SDX chloride vs. placebo.

### Secondary PD Endpoints:

VAS Drug Liking Eₘₐₓ: SDX chloride vs. phentermine.
SDX chloride vs. Focalin® XR, phentermine and placebo for Take Drug Again and Overall Drug Liking at 12 hours postdose.

### Exploratory PD Endpoints:

SDX chloride vs. Focalin® XR, phentermine and placebo for Eₘₐₓ of Feeling High, Good Effects, Bad Effects, Any Effects, and Drowsiness/Alertness VAS scores, and for Take Drug Again and Overall Drug Liking at 24 hours postdose.

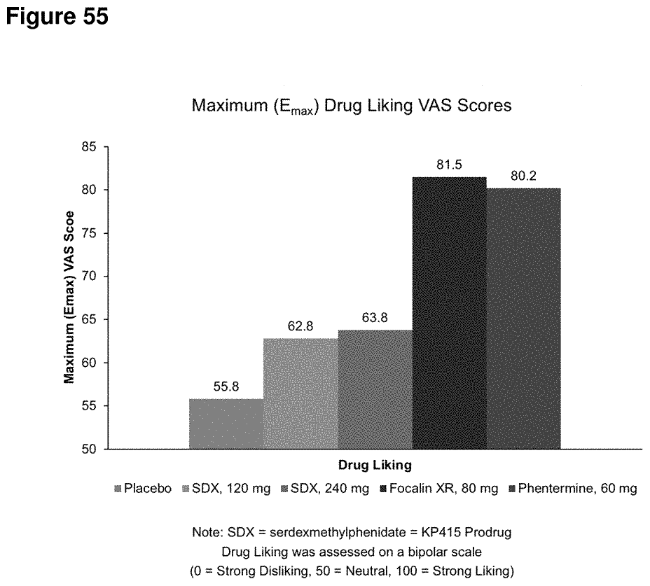

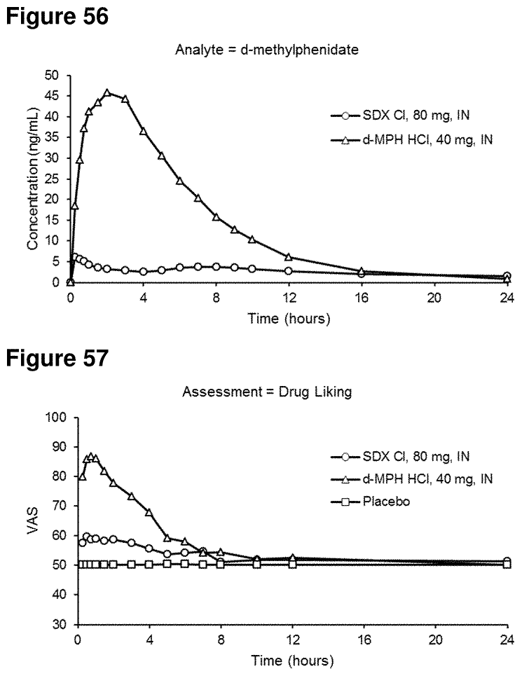

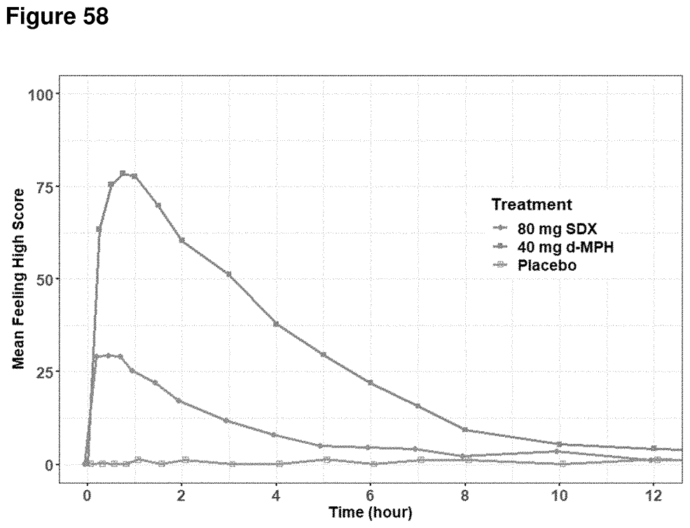

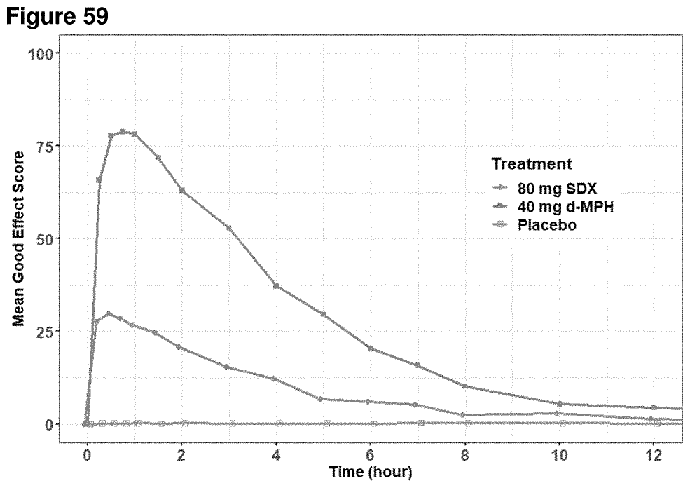

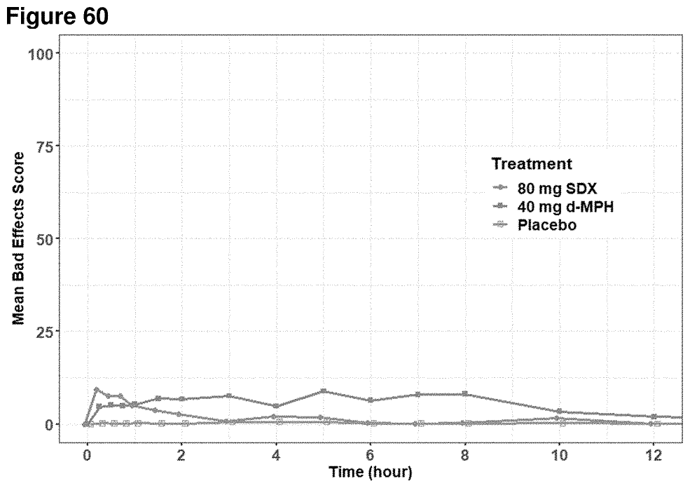

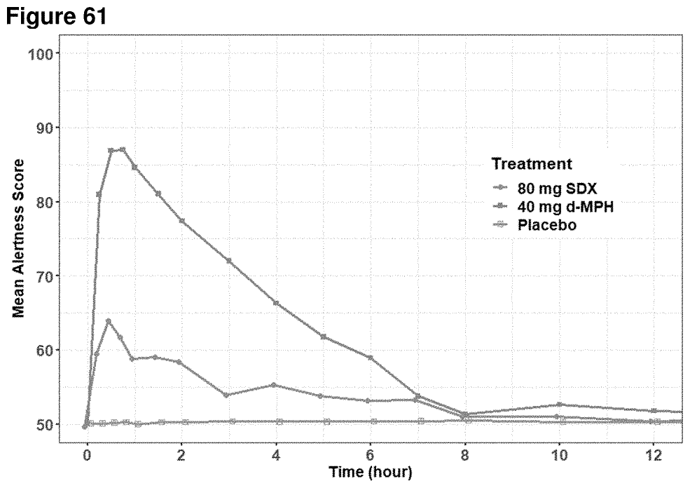

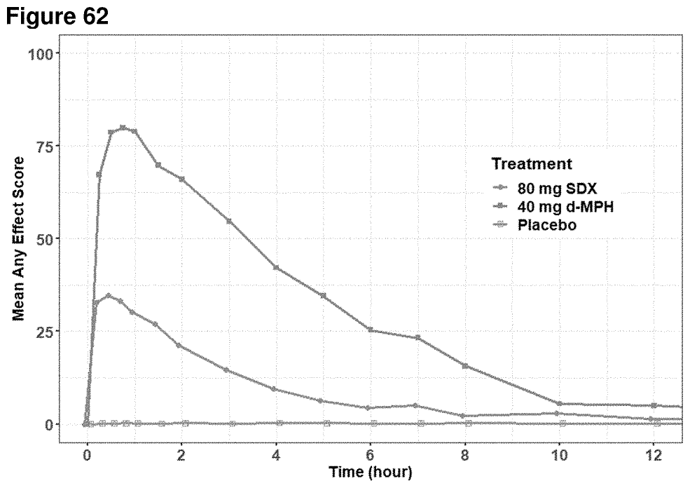

Forty-five (45) subjects completed the study. Comparisons of Drug Liking Eₘₐₓ between each positive control (Focalin® XR and Phentermine) and placebo were conducted for study validity demonstrating absolute abuse potential of the positive controls and demonstrating that subjects were able to discriminate between positive control and placebo. Absolute abuse potential is a comparison with placebo. Relative Abuse Potential is a comparison of the test product (i.e., SDX chloride) to an active control. Assessment results are shown in the Tables below. Mean Drug Liking Eₘₐₓ scores are shown graphically in FIG. 55.

TABLE 22

| | |
|---|---|
| Liking Eₘₐₓ Scores | Drug Liking Eₘₐₓ^{a} |

| Treatment | Mean | Median |
|---|---|---|
| A | 62.8 | 53.0 |
| B | 63.8 | 59.0 |
| C | 81.5 | 82.0 |
| D | 80.2 | 84.0 |
| E | 55.8 | 51.0 |

^{a}assessed on a bipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 23

Mean Difference
Drug Liking^{a}

| Comparison | Margin | Eₘₐₓ | 95% CI^{b} |
|---|---|---|---|
| C vs E | 15 | 25.0 | (20.7, ∞) |
| C vs A | 10 | 18.2 | (13.9, ∞) |
| C vs B | 10 | 16.7 | (12.4, ∞) |
| A vs E | 11 | 6.8 | (−∞, 11.2) |
| B vs E | 11 | 8.3 | (−∞, 12.6) |
| D vs E | 10 | 22.3 | (17.9, ∞) |
| D vs A | 10 | 15.5 | (11.1, ∞) |
| D vs B | 10 | 14.0 | (9.6, ∞) |

^{a}assessed on a bipolar VAS
^{b}95% confidence interval of 1-sided test
A = serdexmethylphenidate chloride, 120 mg
B = serdexmethylphenidate chloride, 240 mg
C = Focalin ® XR, 80 mg
D = phentermine hydrochloride, 60 mg
E = Placebo

Statistical analyses of the primary endpoint, Drug Liking Eₘₐₓ, indicated that Eₘₐₓ was statistically significantly higher for Focalin® XR (Treatment C) vs. placebo (Treatment E) and Phentermine (Treatment D) vs. placebo (Treatment E) by at least a 15-point and 10-point margin, respectively, thus demonstrating study validity. Both 120 mg (Treatment A) and 240 mg (Treatment B) SDX chloride produced mean responses of Drug Liking Eₘₐₓ that were statistically significantly lower compared to the positive control Focalin® XR by at least a 10-point margin. The 120 mg SDX chloride produced mean responses of Drug Liking Eₘₐₓ that were statistically significantly lower by at least a 10-point margin compared to the positive control phentermine. Although mean Drug Liking Eₘₐₓ of 240 mg SDX chloride was numerically lower compared to phentermine, the difference was not statistically greater than 10 points. Both 120 mg and 240 mg SDX chloride produced mean responses of Drug Liking Eₘₐₓ which were numerically similar and as a result not statistically non-inferior to placebo within an 11-point margin. Overall, 120 mg SDX chloride produced mean responses of Drug Liking Eₘₐₓ that were statistically lower by at least 10 points compared to 80 mg Focalin® XR and 60 mg phentermine. Mean Drug Liking Eₘₐₓ of 240 mg SDX chloride was also lower by at least 10 points versus 80 mg Focalin® XR but not versus 60 mg phentermine. Mean Drug Liking Eₘₐₓ of 240 mg SDX chloride, however, was still statistically lower compared to 60 mg phentermine. While Drug Liking Eₘₐₓ of 120 and 240 mg of SDX chloride were only slightly higher compared to placebo, they were statistically similar within an 11-point margin to Focalin® XR

TABLE 22
Take Drug Again Eₘₐₓ Scores

Take Drug Again Eₘₐₓ^{a}

| Treatment | Mean | Median |
|---|---|---|
| A | 57.4 | 50.0 |
| B | 56.9 | 50.0 |
| C | 64.6 | 69.0 |
| D | 72.3 | 75.0 |
| E | 55.2 | 50.0 |

^{a}assessed on a bipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 23

| | Mean Difference | | |
|---|---|---|---|
| | Take Drug Again^{a} | | |
| Comparison | Eₘₐₓ | CI^{b} | alpha |
| C vs E | 9.4 | (1.1, 17.6) | 0.05 |
| C vs A | 7.2 | (−2.7, 17.1) | 0.05 |
| C vs B | 7.6 | (−3.8, 19.1) | 0.05 |
| A vs E | 2.2 | (−3.5, 7.8) | 0.1 |
| B vs E | 1.7 | (−3.7, 7.2) | 0.1 |
| D vs E | 17.1 | (9.9, 24.3) | 0.05 |
| D vs A | 14.9 | (7.5, 22.3) | 0.05 |
| D vs B | 15.4 | (8.3, 22.4) | 0.05 |

^{a}assessed on a bipolar VAS
^{b}confidence interval of 2-sided test
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Take Drug Again (TDA) Eₘₐₓ for both doses of SDX chloride was smaller than for Focalin® XR, but the differences were not statistically significant. Without wanting to be bound by any particular theory, these results may be due to subjects experiencing a significant Focalin® rebound effect with Focalin® XR (“crashing” later in the day as supported by Bad Effects scores) that may have influenced the retrospective TDA scores assessed at 12 and 24 hours postdose but not early Drug Liking scores. Mean TDA Eₘₐₓ for both doses of SDX chloride were statistically lower than for phentermine suggesting that subjects would prefer to take again phentermine over SDX. The mean TDA Eₘₐₓ of both doses of SDX chloride was not statistically different from placebo suggesting that subjects did not prefer to take again SDX over

TABLE 24
Overall Drug Liking Eₘₐₓ Scores

Overall Drug Liking Eₘₐₓ^{a}

| Treatment | Mean | Median |
|---|---|---|
| A | 58.5 | 50.0 |
| B | 58.0 | 51.0 |
| C | 63.0 | 71.0 |
| D | 73.4 | 77.0 |
| E | 54.8 | 50.0 |

^{a}assessed on a bipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 25

| | Mean Difference | | |
|---|---|---|---|
| | Overall Drug Liking^{a} | | |
| Comparison | Eₘₐₓ | CI^{b} | alpha |
| C vs E | 8.3 | (0.24, 16.3) | 0.05 |
| C vs A | 4.5 | (−4.4, 13.4) | 0.05 |
| C vs B | 5.0 | (−5.2, 15.1) | 0.05 |
| A vs E | 3.8 | (−1.4, 8.9) | 0.1 |
| B vs E | 3.3 | (−1.2, 7.8) | 0.1 |
| D vs E | 18.6 | (11.5, 25.7) | 0.05 |
| D vs A | 14.9 | (7.1, 22.7) | 0.05 |
| D vs B | 15.3 | (7.1, 23.6) | 0.05 |

^{a}assessed on a bipolar VAS
^{b}confidence interval of 2-sided test
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Overall Drug Liking (ODL) Eₘₐₓ for both doses of SDX chloride was smaller than for Focalin® XR, but the differences were not statistically significant. Again, without wanting to be bound by any particular theory, these results may be due to subjects experiencing a significant rebound effect with Focalin® XR (“crashing” later in the day as supported by Bad Effects scores) that may have influenced the retrospective ODL scores assessed at 12 and 24 hours postdose but not early Drug Liking scores. Mean ODL Eₘₐₓ for both doses of SDX chloride are statistically lower than for phentermine. The mean ODL Eₘₐₓ of both doses of SDX chloride was not statistically different from placebo suggesting that Overall Drug Liking was similar for SDX and placebo.

TABLE 26
Feeling High Eₘₐₓ Scores

Feeling High Eₘₐₓ^{a}

| Treatment | Mean | Median |
|---|---|---|
| A | 25.6 | 7.0 |
| B | 32.1 | 19.0 |
| C | 78.8 | 83.0 |
| D | 65.3 | 76.0 |
| E | 13.0 | 0.0 |

^{a}assessed on a unipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 27

| | Mean Difference | | |
|---|---|---|---|
| | Feeling High^{a} | | |
| Comparison | Eₘₐₓ | CI^{b} | alpha |
| C vs E | 62.8 | (51.2, 74.4) | 0.05 |
| C vs A | 52.4 | (40.7, 64.1) | 0.05 |
| C vs B | 44.0 | (32.3, 55.8) | 0.05 |
| A vs E | 10.4 | (0.55, 20.3) | 0.1 |
| B vs E | 18.8 | (9.0, 28.6) | 0.1 |
| D vs E | 46.4 | (34.7, 58.1) | 0.05 |
| D vs A | 36.0 | (24.3, 47.7) | 0.05 |
| D vs B | 27.7 | (15.9, 39.4) | 0.05 |

^{a}assessed on a unipolar VAS
^{b}confidence interval of 2-sided test
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Feeling High Eₘₐₓ for both doses of SDX chloride was statistically lower compared to Focalin® XR and phentermine. The mean Feeling High Eₘₐₓ of 120 and 240 mg SDX chloride were statistically higher compared to placebo.

TABLE 28
Good Effects Eₘₐₓ Scores

Good Effects Eₘₐₓ^{a}

| Treatment | Mean | Median |
|---|---|---|
| A | 27.0 | 10.0 |
| B | 30.8 | 15.0 |
| C | 75.7 | 85.0 |
| D | 66.2 | 75.0 |
| E | 13.0 | 0.0 |

^{a}assessed on a unipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 29

| | Mean Difference | | |
|---|---|---|---|
| | Good Effects^{a} | | |
| Comparison | Eₘₐₓ | CI^{b} | alpha |
| C vs E | 60.3 | (48.8, 71.9) | 0.05 |
| C vs A | 48.2 | (36.6, 59.8) | 0.05 |
| C vs B | 42.9 | (31.3, 54.5) | 0.05 |
| A vs E | 12.1 | (2.3, 21.9) | 0.1 |
| B vs E | 17.4 | (7.7, 27.1) | 0.1 |
| D vs E | 47.6 | (36.0, 59.2) | 0.05 |
| D vs A | 35.5 | (23.9, 47.1) | 0.05 |
| D vs B | 30.2 | (18.5, 41.8) | 0.05 |

^{a}assessed on a unipolar VAS
^{b}confidence interval of 2-sided test
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Good Effects Eₘₐₓ for both doses of SDX chloride was statistically lower compared to Focalin® XR and phentermine. The mean Good Effects Eₘₐₓ of 120 and 240 mg SDX chloride were statistically higher compared to placebo.

TABLE 30
Bad Effects Eₘₐₓ Scores

Bad Effects Eₘₐₓ^{a}

| Treatment | Mean | Median |
|---|---|---|
| A | 6.04 | 0.0 |
| B | 13.4 | 0.0 |
| C | 33.8 | 21.0 |
| D | 17.9 | 7.0 |
| E | 4.8 | 0.0 |

^{a}assessed on a unipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 31

| | Mean Difference | | |
|---|---|---|---|
| | Bad Effects^{a} | | |
| Comparison | Eₘₐₓ | CI^{b} | alpha |
| C vs E | 29.0 | (18.4, 39.7) | 0.05 |
| C vs A | 27.8 | (17.2, 38.3) | 0.05 |
| C vs B | 20.5 | (9.6, 31.4) | 0.05 |
| A vs E | 1.3 | (−4.1, 6.6) | 0.10 |
| B vs E | 8.6 | (2.1, 15.1) | 0.10 |
| D vs E | 8.0c | (3.5, 15.0) | 0.05 |
| D vs A | 11.9 | (3.8, 20.0) | 0.05 |
| D vs B | 4.6 | (−4.8, 13.9) | 0.05 |

^{a}assessed on a unipolar VAS
^{b}confidence interval of 2-sided test
cmedian difference
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Bad Effects Eₘₐₓ of 120 mg SDX chloride was statistically lower compared to Focalin® XR and phentermine. The mean Bad Effects Eₘₐₓ of 240 mg SDX chloride was also statistically lower compared to Focalin® XR but not phentermine. The mean Bad Effects Eₘₐₓ of 120 mg SDX chloride was not statistically different from placebo suggesting that subjects did not experience significant negative or bad effects with an oral dose of 120 mg SDX chloride. The mean Bad Effects Eₘₐₓ of 240 mg SDX chloride was statistically higher compared to placebo, but numerically and statistically similar to phentermine.

TABLE 32
Feeling Alert/Drowsy Eₘₐₓ Scores

Feeling Alert/Drowsy Eₘₐₓ^{a}

| Treatment | Mean | Median |
|---|---|---|
| A | 64.4 | 54.0 |
| B | 67.4 | 64.0 |
| C | 86.3 | 87.0 |
| D | 81.4 | 84.0 |
| E | 56.1 | 51.0 |

^{a}assessed on a bipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 33

| | Mean Difference | | |
|---|---|---|---|
| | Feeling Alert/Drowsy^{a} | | |
| Comparison | Eₘₐₓ | CI^{b} | alpha |
| C vs E | 29.5 | (24.3, 34.6) | 0.05 |
| C vs A | 21.7 | (16.5, 26.9) | 0.05 |
| C vs B | 18.2 | (13.0, 23.5) | 0.05 |
| A vs E | 7.7 | (3.4, 12.1) | 0.1 |
| B vs E | 11.2 | (6.9, 15.6) | 0.1 |
| D vs E | 23.5 | (18.3, 28.7) | 0.05 |
| D vs A | 15.7 | (10.5, 20.9) | 0.05 |
| D vs B | 12.2 | (7.0, 17.5) | 0.05 |

^{a}assessed on a bipolar VAS
^{b}confidence interval of 2-sided test
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Feeling Alert/Drowsy Eₘₐₓ for both doses of SDX chloride was statistically lower compared to Focalin® XR and phentermine. The mean Feeling Alert/Drowsy Eₘₐₓ for both doses of SDX chloride was statistically higher compared to placebo suggesting that subjects felt somewhat more alert after oral doses of 120 and 240 mg of SDX chloride but not as much as after oral doses of 80 mg Focalin® XR and 60 mg phentermine.

TABLE 34
Any Effects Eₘₐₓ Scores

Any Effects Eₘₐₓ^{a}

| Treatment | Mean | Median |
|---|---|---|
| A | 28.8 | 14.0 |
| B | 37.0 | 23.0 |
| C | 80.4 | 90.0 |
| D | 69.2 | 74.0 |
| E | 15.6 | 0.0 |

^{a}assessed on a unipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

TABLE 35

| | Mean Difference | | |
|---|---|---|---|
| | Any Effects^{a} | | |
| Comparison | Eₘₐₓ | CI^{b} | alpha |
| C vs E | 62.1 | (49.9, 74.2) | 0.05 |
| C vs A | 51.0 | (38.8, 63.3) | 0.05 |
| C vs B | 41.6 | (29.3, 53.9) | 0.05 |
| A vs E | 11.0 | (0.72, 21.4) | 0.1 |
| B vs E | 20.5 | (10.2, 30.7) | 0.1 |
| D vs E | 48.5 | (36.2, 60.7) | 0.05 |
| D vs A | 37.4 | (25.2, 49.7) | 0.05 |
| D vs B | 28.0 | (15.7, 40.3) | 0.05 |

^{a}assessed on a unipolar VAS
A = serdexmethylphenidate chloride, 120 mg
B = serdexmethylphenidate chloride, 240 mg
C = Focalin ® XR, 80 mg
D = phentermine hydrochloride, 60 mg
E = Placebo

The mean Any Effects Eₘₐₓ for both doses of SDX chloride was statistically lower compared to Focalin® XR and phentermine. The mean Feeling High Eₘₐₓ of 120 mg SDX chloride was not statistically different from placebo suggesting that subjects did not feel any significant drug effects with an oral dose of 120 mg SDX chloride. The mean Any Effects Eₘₐₓ of 240 mg SDX chloride was statistically higher compared to placebo.

In the present specification, use of the singular includes the plural except where specifically indicated. Further aspects and embodiments of the present technology are described in the paragraphs below.

In some embodiments, the present technology provides a composition comprising serdexmethylphenidate wherein the composition exhibits a substantially similar mean Overall Drug Liking (“ODL”) Eₘₐₓ when compared to Focalin® XR following oral administration. In some embodiments, the composition comprises a dose of 120 mg or less of serdexmethylphenidate chloride and exhibits a substantially similar mean Overall Drug Liking (“ODL”) Eₘₐₓ with the mean difference of about 4.5 having a 95% Confidence Interval of about (−4.4, 13.4) when compared to 80 milligrams of Focalin® XR per dose. In alternative embodiments, the composition comprises a dose of 240 mg or less of serdexmethylphenidate chloride and exhibits a substantially similar mean Overall Drug Liking (“ODL”) Eₘₐₓ with the mean difference of about 5.0 having a 95% Confidence Interval of about (−5.2, 15.1) when compared to 80 milligrams of Focalin® XR per dose.

In some embodiments, the present technology provides a composition comprising serdexmethylphenidate wherein the composition exhibits a substantially lower mean Overall Drug Liking (“ODL”) Eₘₐₓ when compared to 60 milligrams of phentermine hydrochloride per dose following oral administration. In some embodiments, the composition comprises a dose of 120 mg or less of serdexmethylphenidate chloride and exhibits a substantially lower mean Overall Drug Liking (“ODL”) Eₘₐₓ with the mean difference of about 14.9 having a 95% Confidence Interval of about (7.1, 22.7) when compared to 60 milligrams of phentermine hydrochloride per dose. In alternative embodiments, the composition comprises a dose of 240 mg or less of serdexmethylphenidate chloride and exhibits a substantially lower mean Overall Drug Liking (“ODL”) Eₘₐₓ with the mean difference of about 15.3 having a 95% Confidence Interval of about (7.1, 23.6) when compared to 60 milligrams of phentermine hydrochloride per dose.

In some embodiments, the present technology provides a composition comprising serdexmethylphenidate wherein the composition exhibits a substantially lower mean Drug Liking (“DL”) Eₘₐₓ when compared to Focalin® XR following oral administration. In some embodiments, the composition comprises a dose of 120 mg or less of serdexmethylphenidate chloride and exhibits a statistically significantly lower than twice the maximum daily clinical dose of Focalin® XR (2×40 mg=80 mg). In some embodiments, the composition comprises a dose of 120 mg or less of serdexmethylphenidate chloride and exhibits a mean Drug Liking (“DL”) Eₘₐₓ that is substantially lower by a margin of at least about 10 when compared to 80 mg of Focalin® XR per dose. In other embodiments, the composition comprises a dose of 120 mg or less of serdexmethylphenidate chloride and exhibits a mean difference of at least about 18.2 in Drug Liking (“DL”) Eₘₐₓ compared to 80 mg of Focalin® XR with a lower limit of the 95% Confidence Interval of about 13.9 indicating that the mean Drug Liking Eₘₐₓ is substantially lower for serdexmethylphenidate compared to Focalin® XR by a margin of up to about 13.9. In alternative embodiments, the composition comprises a dose of 240 mg or less of serdexmethylphenidate chloride and exhibits a statistically significantly lower than twice the maximum daily clinical dose of Focalin® XR (2×40 mg=80 mg). In alternative embodiments, the composition comprises a dose of 240 mg or less of serdexmethylphenidate and exhibits a mean Drug Liking (“DL”) Eₘₐₓ that is substantially lower by a margin of at least about 10 when compared to 80 mg of Focalin® XR per dose. In other embodiments, the composition comprises a dose of 240 mg or less of serdexmethylphenidate chloride and exhibits a mean difference of at least about 16.7 in Drug Liking (“DL”) Eₘₐₓ compared to 80 mg of Focalin® XR with a lower limit of the 95% Confidence Interval of about 12.4 indicating that the mean Drug Liking Eₘₐₓ is substantially lower for serdexmethylphenidate compared to Focalin® XR by a margin of up to about 12.4.

In some embodiments, the present technology provides a composition comprising serdexmethylphenidate wherein the composition exhibits a substantially lower mean Drug Liking (“DL”) Eₘₐₓ when compared to 60 mg of phentermine hydrochloride per dose following oral administration. In some embodiments, the composition comprises a dose of 120 mg or less of serdexmethylphenidate chloride and exhibits a statistically significantly lower mean Drug Liking (“DL”) Eₘₐₓ compared to twice the maximum daily clinical dose of phentermine (2×30 mg=60 mg), a schedule IV controlled substance. In some embodiments, the composition comprises a dose of 120 mg or less of serdexmethylphenidate chloride and exhibits a mean Drug Liking (“DL”) Eₘₐₓ that is substantially lower by a margin of at least about 10 when compared to 60 mg of phentermine hydrochloride per dose. In other embodiments, the composition comprises a dose of 120 mg or less of serdexmethylphenidate chloride and exhibits a mean difference of at least about 15.5 in Drug Liking (“DL”) Eₘₐₓ compared to 60 mg of phentermine hydrochloride with a lower limit of the 95% Confidence Interval of about 11.1 indicating that the mean Drug Liking Eₘₐₓ is substantially lower for serdexmethylphenidate compared to phentermine by a margin of up to about 11.1. In alternative embodiments, the composition comprises a dose of 240 mg or less of serdexmethylphenidate chloride and exhibits a mean difference of at least about 14.0 in Drug Liking (“DL”) Eₘₐₓ compared to 60 mg of phentermine hydrochloride with a lower limit of the 95% Confidence Interval of about 9.6 indicating that the mean Drug Liking Eₘₐₓ is substantially lower for serdexmethylphenidate compared to phentermine by a margin of up to about 9.6.

In some embodiments, the present technology provides a composition comprising serdexmethylphenidate, wherein the composition exhibits a substantially similar mean Take Drug Again (“TDA”) Eₘₐₓ when compared to Focalin® XR following oral administration. In some embodiments, the composition comprises a dose of 120 mg or less of serdexmethylphenidate chloride and exhibits a substantially similar mean Take Drug Again Eₘₐₓ with the mean difference having a 95% Confidence Interval of about (−2.7, 17.1) when compared to 80 milligrams of Focalin® XR per dose. In alternative embodiments, the composition comprises a dose of 240 mg or less of serdexmethylphenidate chloride and exhibits a substantially similar mean Take Drug Again Eₘₐₓ with the mean difference having a 95% Confidence Interval of about (−3.8, 19.1) when compared to 80 milligrams of Focalin® XR per dose.

In some embodiments, the present technology provides a composition comprising serdexmethylphenidate, wherein the composition exhibits a substantially lower mean Take Drug Again (“TDA”) Eₘₐₓ when compared to 60 milligrams of phentermine hydrochloride per dose following oral administration. In some embodiments, the composition comprises a dose of 120 mg or less of serdexmethylphenidate chloride and exhibits a substantially lower mean Take Drug Again Eₘₐₓ with the mean difference having a 95% Confidence Interval of about (7.5, 22.3) when compared to 60 milligrams of phentermine hydrochloride per dose. In alternative embodiments, the composition comprises a dose of 240 mg or less of serdexmethylphenidate chloride and exhibits a substantially lower mean Take Drug Again Eₘₐₓ with the mean difference of 15.4 having a 95% Confidence Interval of about (8.3, 22.4) when compared to 60 milligrams of phentermine hydrochloride per dose.

In some embodiments, the present technology provides a composition comprising serdexmethylphenidate, wherein the composition may have lower oral abuse potential compared to Focalin® XR (d-methylphenidate extended release capsules), a schedule II controlled substance, when administered at oral doses up to 1.5 times higher than Focalin® XR on a molar basis.

### Example 5: Intranasal Abuse Potential and Pharmacokinetic Study

This was a Phase 1, randomized, double-blind, single dose, active-and placebo-controlled, 3-treatment, 3-period, 6-sequence crossover study evaluating the abuse potential and pharmacokinetics of 80 mg serdexmethylphenidate (SDX) chloride, 40 mg d-methylphenidate hydrochloride, and placebo, after intranasal administration in healthy, nondependent, recreational stimulant users with intranasal insufflation experience. The study consisted of a Screening Period, an in-clinic Drug Discrimination Phase, a Treatment Phase, and a Follow-Up Visit.

Subjects who successfully completed the Screening Period returned to the clinic to complete the Drug Discrimination Phase. The Drug Discrimination Test was performed to ensure that subjects can differentiate between the effects of a single dose of d-methylphenidate hydrochloride and placebo, administered intranasally. Subjects who successfully completed the Drug Discrimination Phase remained as inpatients to enter the Treatment Phase. The Drug Discrimination Phase was a double-blind, intranasal, single-dose, 2-treatment, 2-period, 2-sequence, randomized, crossover design. Subjects received single intranasal doses of the following treatments separated by a 48-hour washout period:
Treatment X: 40 mg d-methylphenidate hydrochloride powder mixed with 40 mg microcrystalline cellulose (MCC).
Treatment Y: 80 mg matching placebo powder.

All subjects were required to fast for at least 8 hours prior to each dose of study drug until approximately 4 hours after each dose. Abuse potential measures and pharmacokinetic samples were collected at different times after the administration of study drug. Subjects who successfully completed the Drug Discrimination Phase and who qualified for the Treatment Phase remained as inpatients to enter the Treatment Phase. After a washout period of approximately 72 hours after the last dose of study drug in the Drug Discrimination Phase, subjects who were eligible to continue in the Treatment Phase were randomized into the Treatment Phase in a 1:1:1 ratio to receive 3 different treatments in a double-blind, crossover design separated by a minimum 96-hour washout period as follows:
Treatment A: 80 mg serdexmethylphenidate chloride powder (test product).
Treatment B: 40 mg d-methylphenidate hydrochloride powder mixed with 40 mg microcrystalline cellulose (control product).
Treatment C: 80 mg microcrystalline cellulose (matching placebo powder).

To ensure blinding, the d-methylphenidate hydrochloride dose was mixed with an appropriate amount of microcrystalline cellulose to create a volume that was approximately the same as the volume of 80 mg serdexmethylphenidate chloride powder. The placebo dose consisted of 80 mg of microcrystalline cellulose to create the same volume.

On dosing days in the Treatment Phase, blood samples were collected for the measurement of the plasma concentrations of SDX, d-methylphenidate (d-MPH), 1-methylphenidate (1-MPH) and ritalinic acid at predose and at 0.25, 0.5, 0.75, 1, 1.5, 2, 3, 4, 5, 6, 7, 8, 9, 10, 12, 16, 24, 36, and 48 hours ±5 minutes after each dose of study drug. Pharmacodynamic assessments included VAS assessments of Drug Liking, Good Effects, Bad Effects, Any Effects, Feeling High, Drowsiness/Alertness, Take Drug Again, and Overall Drug Liking. All VAS pharmacodynamic assessments except Take Drug Again and Overall Drug Liking were performed at 0.25, 0.5, 0.75, 1, 1.5, 2, 3, 4, 5, 6, 7, 8, 10, 12, 24 hours ±5 minutes postdose. In addition, predose assessments of Feeling High, and Drowsiness/Alertness were collected. The Take Drug Again and Overall Drug Liking VAS assessments were performed at 12 and 24 hours ±5 minutes postdose. Ease of Nasal Insufflation scores were completed within 5 minutes after the completion of each intranasal drug administration during the Treatment Phase.

Pharmacodynamic endpoint VAS scales:
Bipolar VAS “At this Moment” Drug Liking (postdose assessments).
Unipolar VAS “At this Moment” Good Effects, Bad Effects, Any Effects (postdose assessments).
Unipolar VAS “At this Moment” Feeling High (predose and postdose assessments).
Bipolar VAS Drowsiness/Alertness (predose and postdose assessments).
Bipolar VAS Take Drug Again at 12 and 24 hours postdose.
Bipolar VAS Overall Drug Liking at 12 and 24 hours postdose.
Unipolar Ease of Nasal Insufflation (postdose assessment).

### Primary Pharmacodynamic Endpoint:

Drug Liking Eₘₐₓ: d-methylphenidate hydrochloride vs. placebo, serdexmethylphenidate chloride vs. d-methylphenidate hydrochloride, and serdexmethylphenidate chloride vs. placebo.

### Secondary PD Endpoints:

Serdexmethylphenidate chloride vs. d-methylphenidate hydrochloride and serdexmethylphenidate chloride vs. Placebo for Take Drug Again and Overall Drug Liking at 12 hours post-dosing.

### Exploratory PD Endpoints:

Serdexmethylphenidate chloride vs. d-methylphenidate hydrochloride and serdexmethylphenidate chloride vs. Placebo for Eₘₐₓ of High, Good Effects, Bad Effects, Any Effects, and Drowsiness/Alertness VAS scores; and for Take Drug Again and Overall Drug Liking at 24 hours post-dosing; and for Ease of Nasal Insufflation (score at postdose assessment).

Forty-five (45) subjects completed the study. Comparisons of Drug Liking Eₘₐₓ between each positive control (SDX chloride and d-methylphenidate hydrochloride) and placebo were conducted for study validity demonstrating absolute abuse potential of the positive controls and demonstrating that subjects were able to discriminate between positive control and placebo. Absolute abuse potential is a comparison with placebo. Relative Abuse Potential is a comparison of the test product (i.e., SDX chloride) to an active control. Assessment results are shown in the Tables below. Mean Drug Liking Eₘₐₓ scores are shown graphically in FIG. 55.

TABLE 36

| | Mean Eₘₐₓ Scores | | | | |
|---|---|---|---|---|---|
| | | Take | Overall | | Feeling |
| | Drug | Drug | Drug | Feeling | Drowsy/ |
| Treatment | Liking^{a} | Again^{a} | Liking^{a} | High^{b} | Alert^{a} |
| A | 71.0 | 60.0 | 61.8 | 43.4 | 71.2 |
| B | 93.2 | 80.3 | 81.0 | 88.0 | 93.4 |
| C | 51.1 | 49.2 | 50.3 | 2.7 | 51.1 |

^{a}assessed on a bipolar VAS
^{b}assessed on a unipolar VAS
A = serdexmethylphenidate chloride, 80 mg
B = d-methylphenidate hydrochloride, 40 mg
C = Placebo

TABLE 37

Mean Eₘₐₓ Scores

| | Good | Bad | Any | Ease of Nasal |
|---|---|---|---|---|
| Treatment | Effects^{a} | Effects^{a} | Effect^{a} | Insufflation^{a} |
| A | 44.9 | 14.9 | 49.4 | 65.8 |
| B | 90.6 | 18.6 | 89.2 | 18.1 |
| C | 0.7 | 1.6 | 0.6 | 6.9 |

^{a}assessed on a unipolar VAS
A = serdexmethylphenidate chloride, 80 mg
B = d-methylphenidate hydrochloride, 40 mg
C = Placebo

TABLE 38

| | Median Eₘₐₓ Scores | | | | |
|---|---|---|---|---|---|
| | | Take | Overall | | Feeling |
| | Drug | Drug | Drug | Feeling | Drowsy/ |
| Treatment | Liking^{a} | Again^{a} | Liking^{a} | High^{b} | Alert^{a} |
| A | 71 | 56 | 58 | 36 | 71 |
| B | 100 | 95 | 90 | 97 | 100 |
| C | 51 | 50 | 50 | 0 | 51 |

^{a}assessed on a bipolar VAS
^{b}assessed on a unipolar VAS
A = serdexmethylphenidate chloride, 80 mg
B = d-methylphenidate hydrochloride, 40 mg
C = Placebo

TABLE 39

Median Eₘₐₓ Scores

| | Good | Bad | Any | Ease of Nasal |
|---|---|---|---|---|
| Treatment | Effects^{a} | Effects^{a} | Effect^{a} | Insufflation^{a} |
| A | 42 | 1 | 47 | 71 |
| B | 97 | 2 | 100 | 7 |
| C | 0 | 0 | 0 | 0 |

^{a}assessed on a unipolar VAS
A = serdexmethylphenidate chloride, 80 mg
B = d-methylphenidate hydrochloride, 40 mg
C = Placebo

TABLE 40

| | Mean Take Drug Again^{a} | | Mean Overall Drug Liking^{a} | |
|---|---|---|---|---|
| Treatment | 12 hours | 24 hours | 12 hours | 24 hours |
| A | 56.7 | 55.8 | 60.4 | 59.0 |
| B | 78.2 | 77.4 | 79.6 | 76.3 |
| C | 49.1 | 49.1 | 50.1 | 50.2 |

^{a}assessed on a bipolar VAS
A = serdexmethylphenidate chloride, 80 mg
B = d-methylphenidate hydrochloride, 40 mg
C = Placebo

TABLE 41

| | Median Take Drug Again^{a} | | Median Overall Drug Liking^{a} | |
|---|---|---|---|---|
| Treatment | 12 hours | 24 hours | 12 hours | 24 hours |
| A | 51 | 50 | 58 | 55 |
| B | 91 | 94 | 87 | 82 |
| C | 50 | 50 | 50 | 50 |

^{a}assessed on a bipolar VAS
A = serdexmethylphenidate chloride, 80 mg
B = d-methylphenidate hydrochloride, 40 mg
C = Placebo

TABLE 42

Mean Difference
Drug Liking^{a}

| Treatment | Eₘₐₓ | 95% CI^{b} |
|---|---|---|
| B vs A | 22.3 | (17.3, ∞) |
| A vs C | 19.9 | (−∞, 24.6) |

^{a}assessed on a bipolar VAS
^{b}95% confidence interval of 1-sided test
A = serdexmethylphenidate chloride, 80 mg
B = d-methylphenidate hydrochloride, 40 mg
C = Placebo

TABLE 43

Median Difference
Drug Liking^{a}

| Treatment | Eₘₐₓ | 95% CI^{b} |
|---|---|---|
| B vs C | 45 | (41.0, ∞) |

^{a}assessed on a bipolar VAS
^{b}95% confidence interval of 1-sided test
A = serdexmethylphenidate chloride, 80 mg
B = d-methylphenidate hydrochloride, 40 mg
C = Placebo

Statistical analyses indicated that Drug Liking Eₘₐₓ was statistically higher by at least a 15-point margin for d-methylphenidate hydrochloride vs placebo and by at least a 10-point margin for d-methylphenidate hydrochloride vs serdexmethylphenidate chloride. Drug Liking Eₘₐₓ of serdexmethylphenidate chloride was numerically higher vs placebo and not statistically non-inferior to placebo within an 11-point margin.

TABLE 44

| | Mean Difference | | |
|---|---|---|---|
| | Take Drug Again^{a} | | |
| Treatment | 12 hours | 24 hours | Eₘₐₓ |
| B vs C | 29.1 | 28.3 | 31.1 |
| B vs A | 21.5 | 21.5 | 20.3 |
| A vs C | 7.6 | 6.7 | 10.8 |

^{a}assessed on a bipolar VAS
A = serdexmethylphenidate chloride, 80 mg
B = d-methylphenidate hydrochloride, 40 mg
C = Placebo

Take Drug Again Eₘₐₓ was statistically higher for d-methylphenidate hydrochloride vs placebo, d-methylphenidate hydrochloride vs serdexmethylphenidate chloride, and for serdexmethylphenidate chloride vs placebo. At 12 and 24 hours, Take Drug Again was statistically higher for d-methylphenidate hydrochloride vs placebo and d-methylphenidate hydrochloride vs serdexmethylphenidate chloride at a significance level of alpha=0.05. Take Drug Again was statistically higher for serdexmethylphenidate chloride vs placebo at a significance level of alpha=0.1 at 12 hours but was statistically similar for serdexmethylphenidate chloride vs placebo at a significance level of alpha=0.1 at 24 hours.

TABLE 45

| | Mean Difference | | |
|---|---|---|---|
| | Overall Drug Liking^{a} | | |
| Treatment | 12 hours | 24 hours | Eₘₐₓ |
| B vs A | 19.2 | 17.3 | 19.3 |
| A vs C | 10.3 | 8.8 | 11.5 |

^{a}assessed on a bipolar VAS
A = serdexmethylphenidate chloride, 80 mg
B = d-methylphenidate hydrochloride, 40 mg
C = Placebo

TABLE 46

| | Mean Difference |
|---|---|
| | Overall Drug Liking^{a} |
| Treatment | 24 hours |
| B vs C | 26.1 |

^{a}assessed on a bipolar VAS
A = serdexmethylphenidate chloride, 80 mg
B = d-methylphenidate hydrochloride, 40 mg
C = Placebo

TABLE 47

Median Difference
Overall Drug Liking^{a}

| Treatment | 12 hours | Eₘₐₓ |
|---|---|---|
| B vs C | 33.5 | 35 |

^{a}assessed on a bipolar VAS
A = serdexmethylphenidate chloride, 80 mg
B = d-methylphenidate hydrochloride, 40 mg
C = Placebo

Overall Drug Liking at 12 hours, 24 hours, and Overall Drug Liking Eₘₐₓ were statistically higher for d-methylphenidate hydrochloride vs placebo, d-methylphenidate hydrochloride vs serdexmethylphenidate chloride, and for serdexmethylphenidate chloride vs placebo.

TABLE 48

| | Mean Difference |
|---|---|
| | Feeling High^{a} |
| Treatment | Eₘₐₓ |
| B vs C | 85.2 |
| B vs A | 44.5 |
| A vs C | 40.7 |

^{a}assessed on a unipolar VAS
A = serdexmethylphenidate chloride, 80 mg
B = d-methylphenidate hydrochloride, 40 mg
C = Placebo

Feeling High Eₘₐₓ was statistically higher for d-methylphenidate hydrochloride vs placebo, d-methylphenidate hydrochloride vs serdexmethylphenidate chloride, and for serdexmethylphenidate chloride vs placebo.

TABLE 49

| | Mean Difference |
|---|---|
| | Good Effects^{a} |
| Treatment | Eₘₐₓ |
| B vs C | 90.0 |
| B vs A | 45.7 |
| A vs C | 44.2 |

^{a}assessed on a unipolar VAS
A = serdexmethylphenidate chloride, 80 mg
B = d-methylphenidate hydrochloride, 40 mg
C = Placebo

Good Effects Eₘₐₓ was statistically higher for d-methylphenidate hydrochloride vs placebo, d-methylphenidate hydrochloride vs serdexmethylphenidate chloride, and for serdexmethylphenidate chloride vs placebo.

TABLE 50

| | Median Difference |
|---|---|
| | Bad Effects^{a} |
| Treatment | Eₘₐₓ |
| B vs C | 10.5 |
| B vs A | 0 |
| A vs C | 9.5 |

^{a}assessed on a unipolar VAS
A = serdexmethylphenidate chloride, 80 mg
B = d-methylphenidate hydrochloride, 40 mg
C = Placebo

Bad Effects Eₘₐₓ was statistically higher for d-methylphenidate hydrochloride vs placebo and for serdexmethylphenidate chloride vs placebo. Bad Effects Eₘₐₓ was statistically similar for d-methylphenidate hydrochloride vs serdexmethylphenidate chloride

TABLE 51

| | Mean Difference |
|---|---|
| | Feeling Drowsy/Alert^{a} |
| Treatment | Eₘₐₓ |
| B vs C | 42.3 |
| B vs A | 22.2 |
| A vs C | 20.1 |

^{a}assessed on a bipolar VAS
A = serdexmethylphenidate chloride, 80 mg
B = d-methylphenidate hydrochloride, 40 mg
C = Placebo

Feeling Drowsy/Alert Eₘₐₓ was statistically higher for d-methylphenidate hydrochloride vs placebo, d-methylphenidate hydrochloride vs serdexmethylphenidate chloride, and for serdexmethylphenidate chloride vs placebo.

TABLE 52

| | Mean Difference |
|---|---|
| | Any Effect^{a} |
| Treatment | Eₘₐₓ |
| B vs C | 88.6 |
| B vs A | 39.8 |
| A vs C | 48.8 |

^{a}assessed on a unipolar VAS
A = serdexmethylphenidate chloride, 80 mg
B = d-methylphenidate hydrochloride, 40 mg
C = Placebo

Any Effect Eₘₐₓ was statistically higher for d-methylphenidate hydrochloride vs placebo, d-methylphenidate hydrochloride vs serdexmethylphenidate chloride, and for serdexmethylphenidate chloride vs placebo.

TABLE 53

| | Mean Difference |
|---|---|
| | Ease of Nasal Insufflation^{a} |
| Treatment | Eₘₐₓ |
| B vs C | 11.3 |
| B vs A | −47.7 |
| A vs C | 59.0 |

^{a}assessed on a unipolar VAS
A = serdexmethylphenidate chloride, 80 mg
B = d-methylphenidate hydrochloride, 40 mg
C = Placebo

Ease of Nasal Insufflation Eₘₐₓ was statistically higher for d-methylphenidate hydrochloride vs placebo and for serdexmethylphenidate chloride vs placebo. Ease of Nasal Insufflation Eₘₐₓ was statistically lower for d-methylphenidate hydrochloride vs serdexmethylphenidate chloride.

Statistically significant reductions in maximal Drug Liking (Eₘₐₓ) for SDX chloride at doses of 80 mg (71 points) when compared to an equimolar dose of d-methylphenidate hydrochloride (40 mg, 93 points) were observed. There was also a statistically significant difference with placebo (51 points), but to a lesser extent than d-methylphenidate hydrochloride. In addition, retrospective endpoints of Take Drug Again (Eₘₐₓ) and Overall Drug Liking (Eₘₐₓ) along with secondary abuse potential endpoints including Feeling High (Eₘₐₓ) and Good Effects (Eₘₐₓ) were statistically significantly lower compared to d-methylphenidate hydrochloride.

TABLE 54
Treatment-Emergent Adverse Events

| | Treatment at Onset of Adverse Event | | |
|---|---|---|---|
| | Placebo | d-MPH HCl, 40 mg | SDX Cl, 80 mg |
| | (N = 48) | (N = 46) | (N = 46) |
| TEAE^{a} | n (%) [E]^{b} | n (%) [E]^{b} | n (%) [E]^{b} |
| Any TEAE | 6 (12.5%) [10] | 46 (100.0%) [148] | 36 (78.3%) [102] |
| Psychiatric disorders | 0 | 41 (89.1%) [54] | 13 (28.3%) [19] |
| Euphoric mood | 0 | 29 (63.0%) [29] | 9 (19.6%) [9] |
| Hypervigilance | 0 | 16 (34.8%) [16] | 6 (13.0%) [6] |
| Anxiety | 0 | 2 (4.3%) [2] | 1 (2.2%) [1] |
| Bruxism | 0 | 2 (4.3%) [2] | 0 |
| Restlessness | 0 | 2 (4.3%) [2] | 0 |
| Agitation | 0 | 0 | 1 (2.2%) [1] |
| Change in sustained attention | 0 | 1 (2.2%) [1] | 0 |
| Claustrophobia | 0 | 0 | 1 (2.2%) [1]^{c} |
| Irritability | 0 | 0 | 1 (2.2%) [1] |
| Obsessive-compulsive | 0 | 1 (2.2%) [1] | 0 |
| disorder | | | |
| Phonophobia (fear of loud | 0 | 1 (2.2%) [1] | 0 |
| noises) | | | |
| Respiratory, thoracic and | 1 (2.1%) [1] | 10 (21.7%) [12] | 25 (54.3%) [42] |
| mediastinal disorders | | | |
| Nasal discomfort | 0 | 3 (6.5%) [3] | 13 (28.3%) [14] |
| Nasal congestion | 0 | 1 (2.2%) [1] | 10 (21.7%) [10] |
| Throat irritation | 0 | 3 (6.5%) [3] | 3 (6.5%) [4] |
| Cough | 1 (2.1%) [1] | 0 | 3 (6.5%) [3]^{d} |
| Rhinorrhoea (runny nose) | 0 | 0 | 4 (8.7%) [4] |
| Dry throat | 0 | 3 (6.5%) [3] | 0 |
| Epistaxis (nosebleed) | 0 | 0 | 3 (6.5%) [3] |
| Upper-airway cough | 0 | 0 | 2 (4.3%) [2] |
| syndrome | | | |
| Hypopnoea (shallow | 0 | 1 (2.2%) [1] | 0 |
| breathing) | | | |
| Nasal dryness | 0 | 0 | 1 (2.2%) [1] |
| Pharyngeal hypoaesthesia | 0 | 1 (2.2%) [1] | 0 |
| (numbness of throat) | | | |
| Sneezing | 0 | 0 | 1 (2.2%) [1]^{c} |
| Cardiac disorders | 1 (2.1%) [1] | 19 (41.3%) [29] | 2 (4.3%) [2] |
| Palpitations | 0 | 11 (23.9%) [11] | 2 (4.3%) [2] |
| Sinus tachycardia | 1 (2.1%) [1] | 7 (15.2%) [7] | 0 |
| Tachycardia | 0 | 8 (17.4%) [9] | 0 |
| Ventricular extrasystoles | 0 | 2 (4.3%) [2] | 0 |
| Nervous system disorders | 2 (4.2%) [2] | 9 (19.6%) [10] | 11 (23.9%) [12] |
| Headache | 2 (4.2%) [2] | 7 (15.2%) [7] | 5 (10.9%) [6] |
| Somnolence | 0 | 2 (4.3%) [2] | 1 (2.2%) [1] |
| Disturbance in attention | 0 | 1 (2.2%) [1] | 1 (2.2%) [1] |
| Dysgeusia | 0 | 0 | 2 (4.3%) [2] |
| Dizziness | 0 | 0 | 1 (2.2%) [1] |
| Head discomfort | 0 | 0 | 1 (2.2%) [1] |
| General disorders and | 1 (2.1%) [1] | 12 (26.1%) [13] | 6 (13.0%) [7] |
| administration site conditions | | | |
| Fatigue | 0 | 3 (6.5%) [3] | 1 (2.2%) [1] |
| Feeling hot | 0 | 3 (6.5%) [3] | 0 |
| Asthenia | 0 | 1 (2.2%) [1] | 1 (2.2%) [1] |
| Energy increased | 0 | 2 (4.3%) [2] | 0 |
| Feeling of relaxation | 0 | 0 | 2 (4.3%) [2] |
| Catheter site haematoma | 0 | 0 | 1 (2.2%) [1]^{c} |
| Catheter site swelling | 0 | 0 | 1 (2.2%) [1]^{c} |
| Chest discomfort | 0 | 1 (2.2%) [1] | 0 |
| Chest pain | 0 | 0 | 1 (2.2%) [1] |
| Peripheral swelling | 0 | 1 (2.2%) [1]^{c} | 0 |
| Pyrexia | 1 (2.1%) [1]^{c} | 0 | 0 |
| Vessel puncture site bruise | 0 | 1 (2.2%) [1]^{c} | 0 |
| Vessel puncture site pain | 0 | 1 (2.2%) [1]^{c} | 0 |
| Gastrointestinal disorders | 2 (4.2%) [2] | 9 (19.6%) [10] | 4 (8.7%) [6] |
| Dry mouth | 0 | 6 (13.0%) [6] | 0 |
| Abdominal pain | 0 | 1 (2.2%) [1] | 2 (4.3%) [2] |
| Nausea | 0 | 1 (2.2%) [1] | 2 (4.3%) [2] |
| Diarrhoea | 0 | 1 (2.2%) [1] | 1 (2.2%) [1] |
| Abdominal distension | 0 | 0 | 1 (2.2%) [1] |
| Defaecation urgency | 0 | 1 (2.2%) [1] | 0 |
| Dyspepsia | 1 (2.1%) [1] | 0 | 0 |
| Vomiting | 1 (2.1%) [1] | 0 | 0 |
| Eye disorders | 0 | 3 (6.5%) [3] | 8 (17.4%) [9] |
| Lacrimation increased | 0 | 0 | 8 (17.4%) [8] |
| Eye irritation | 0 | 1 (2.2%) [1] | 0 |
| Eye pain | 0 | 0 | 1 (2.2%) [1] |
| Photophobia | 0 | 1 (2.2%) [1] | 0 |
| Visual impairment | 0 | 1 (2.2%) [1] | 0 |
| Skin and subcutaneous tissue | 0 | 4 (8.7%) [4] | 4 (8.7%) [4] |
| disorders | | | |
| Hyperhidrosis | 0 | 3 (6.5%) [3] | 2 (4.3%) [2] |
| Skin erosion | 0 | 1 (2.2%) [1] | 1 (2.2%) [1] |
| Ecchymosis (skin | 0 | 0 | 1 (2.2%) [1]^{c} |
| discoloration) | | | |
| Musculoskeletal and connective | 1 (2.1%) [1] | 4 (8.7%) [5] | 1 (2.2%) [1] |
| tissue disorders | | | |
| Back pain | 1 (2.1%) [1]^{c} | 1 (2.2%) [1] | 0 |
| Muscle tightness | 0 | 2 (4.3%) [2] | 0 |
| Arthralgia | 0 | 0 | 1 (2.2%) [1]^{c} |
| Muscle twitching | 0 | 1 (2.2%) [1] | 0 |
| Neck pain | 0 | 1 (2.2%) [1] | 0 |
| Metabolism and nutrition | 0 | 3 (6.5%) [3] | 0 |
| disorders | | | |
| Decreased appetite | 0 | 3 (6.5%) [3] | 0 |
| Vascular disorders | 1 (2.1%) [1] | 2 (4.3%) [2] | 0 |
| Flushing | 0 | 1 (2.2%) [1] | 0 |
| Haematoma | 0 | 1 (2.2%) [1]^{c} | 0 |
| Phlebitis superficial | 1 (2.1%) [1]^{c} | 0 | 0 |
| Reproductive system and breast | 0 | 2 (4.3%) [2] | 0 |
| disorders | | | |
| Dysmenorrhoea (menstrual | 0 | 1 (2.2%) [1]^{c} | 0 |
| cramps) | | | |
| Testis discomfort | 0 | 1 (2.2%) [1] | 0 |
| Infections and infestations | 1 (2.1%) [1] | 0 | 0 |
| Pharyngitis | 1 (2.1%) [1] | 0 | 0 |
| Investigations | 0 | 1 (2.2%) [1] | 0 |
| Blood pressure diastolic | 0 | 1 (2.2%) [1] | 0 |
| increased | | | |

^{a}A Treatment-Emergent Adverse Event (TEAE) is an adverse event which starts or worsens on or after treatment with study drug in the treatment phase.
^{b}n = number of subjects in which the adverse event occurred; percentages are calculated as ratio of number subjects reporting an adverse event and total number of subjects receiving the respective treatment;
E = number of occurrences of the adverse event^{c} respective adverse event (s) is (are) unrelated to study drug^{d} one of the respective adverse events was unrelated to study drug

As shown in Table 54, typical stimulant-related adverse events, such as euphoric mood, hypervigilance, cardiac palpitations and tachycardia occurred more often after intranasal d-methylphenidate hydrochloride compared to intranasal serdexmethylphenidate chloride. Certain respiratory, thoracic, and eye disorders including nasal discomfort, nasal congestion, runny nose (rhinorrhoea), and nosebleed (epistaxis), and eye tearing (increased lacrimation) occurred more often after intranasal serdexmethylphenidate chloride than after intranasal d-methylphenidate hydrochloride. These adverse events are likely a result of serdexmethylphenidate chloride causing more localized irritation in the nose and throat than d-methylphenidate hydrochloride when snorted, and without being bound by a single theory may deter abusers from repeatedly snorting serdexmethylphenidate chloride.

This study indicates that SDX is not readily or effectively converted to the active d-methylphenidate when snorted and, as a result, intranasal administration of SDX results in abuse related effects that are lower compared to d-methylphenidate hydrochloride as measured by multiple endpoints that are commonly used to assess human abuse potential.

### Example 6: Clinical Studies

The efficacy of serdexmethylphenidate (SDX)/d-methylphenidate (d-MPH) was evaluated in a laboratory classroom study conducted in 150 pediatric patients (aged 6 to 12 years) who met Diagnostic and Statistical Manual of Mental Disorders, 5th edition (DSM-5®) criteria for a primary diagnosis of ADHD inattentive, hyperactive-impulsive, or combined inattentive/hyperactive-impulsive subtypes.

Following washout of any previous ADHD medication, the study began with an open-label dose-optimization period (3 weeks) during which patients received flexible-dose SDX/d-MPH 26.2/5.19 mg, 39.2/7.78 mg, or 52.3/10.38 mg administered once daily in the morning. Patients then entered a 1-week randomized, double-blind, parallel group treatment period with the individually optimized dose of SDX/d-MPH or placebo. At the end of this week, raters evaluated the attention and behavior of the patients in a laboratory classroom setting, using the Swanson, Kotkin, Agler, M. Flynn, and Pelham (SKAMP) rating scale. SKAMP is a validated 13-item teacher-rated scale that assesses manifestations of ADHD in a classroom setting. Each item is rated on a 7-point impairment scale.

Efficacy assessments were conducted at pre-dose, and 0.5, 1, 2, 4, 8, 10, 12, and 13 hours post-dosing. The primary efficacy endpoint was the average change from pre-dose in the SKAMP-Combined (attention and deportment) scores over the test day (not including the pre-dose score), comparing SDX/d-MPH to placebo.

The key secondary efficacy endpoints were onset and duration of effect, defined as the first point at which active drug separated from placebo on SKAMP-Combined score changes from pre-dose and the last time point at which active drug separated from placebo on SKAMP-Combined score changes from pre-dose, respectively.

The average change from pre-dose in the SKAMP-Combined scores over the test day was statistically significantly lower (improved) with SDX/d-MPH compared to placebo (Table 55).

TABLE 55
Primary Efficacy Results: SKAMP-Combined Score Changes from
Pre-dose Averaged over Classroom Day in Patients with ADHD.

| | | Mean Pre-dose | LS Mean Change | Placebo- |
|---|---|---|---|---|
| | | Score on | from Pre-Dose over | subtracted |
| Study | Treatment | Classroom Day ^{a} | Classroom Day ^{b} | Difference ^{c} |
| Number | Group | (SD) | (SE) | (95% CI) |
| Study 1 | SDX/d-MPH | 17.0 (8.5) | −3.13 (0.61) | −7.27 (−9.00, |
| | | | | −5.53) |
| | Placebo | 14.9 (9.0) | 4.13 (0.71) | — |

SD: standard deviation; SE: standard error; LS Mean: least-squares mean; CI: confidence interval.
^{a} Visit 6 pre-dose score (Visit 6 occurred at the end of the 1-week randomized, double-blind, parallel group treatment period).
^{b} Visit 6 LS mean change from pre-dose over hours 0.5, 1, 2, 4, 8, 10, 12, and 13.
^{c} Difference (drug minus placebo) in least-squares mean change from pre-dose.

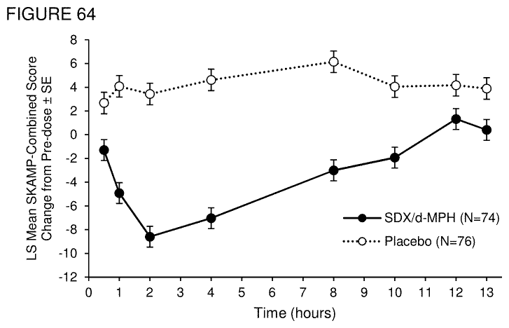

The SKAMP-Combined change scores from pre-dose also demonstrated statistically significant improvement at all time points (0.5. 1, 2, 4, 8, 10, 12, and 13 hours) post-dosing with SDX/d-MPH compared to placebo (FIG. 64).

In some embodiments, the present technology provides a composition comprising serdexmethylphenidate, or a salt thereof, wherein when the composition exhibits a lower mean Drug Liking (“DL”) Eₘₐₓ when compared to d-methylphenidate following intranasal administration of the composition to a human or animal subject. In some embodiments, the composition exhibits a substantially lower mean Drug Liking Eₘₐₓ when compared to d-methylphenidate. In yet another embodiment, the composition comprises an amount of serdexmethylphenidate, or a salt thereof, per dose wherein the composition exhibits a substantially lower mean Drug Liking Eₘₐₓ when compared to 40 mg of d-methylphenidate hydrochloride per dose following intranasal administration of the composition to a human or animal subject. In yet another embodiment, the serdexmethylphenidate salt is serdexmethylphenidate chloride and the amount of serdexmethylphenidate chloride is 80 mg per dose or less. In an alternative embodiment, the serdexmethylphenidate salt is serdexmethylphenidate chloride and the amount of serdexmethylphenidate chloride is at least 80 mg per dose. In another embodiment, the composition exhibits a mean Drug Liking Eₘₐₓ that is substantially lower by a margin of at least 10 when compared to 40 mg of d-methylphenidate hydrochloride per dose.

In some embodiments, the present technology provides a serdexmethylphenidate chloride composition that provides statistically significant reductions in maximal Drug Liking Eₘₐₓ at 80 mg of serdexmethylphenidate chloride when compared to 40 mg d-methylphenidate hydrochloride following intranasal administration of the composition to a human or animal subject.

In some embodiments, the present technology provides a serdexmethylphenidate composition that provides retrospective endpoints of Take Drug Again Eₘₐₓ and Overall Drug Liking Eₘₐₓ that are significantly lower for the serdexmethylphenidate composition when compared to 40 mg d-methylphenidate hydrochloride following intranasal administration of the composition to a human or animal subject. In another embodiment, the serdexmethylphenidate is serdexmethylphenidate chloride and the amount of serdexmethylphenidate chloride is 80 mg per dose or less. In yet another embodiment, the serdexmethylphenidate is serdexmethylphenidate chloride and the amount of serdexmethylphenidate chloride is at least 80 mg per dose.

In some embodiments, the present technology provides a serdexmethylphenidate composition that provides a Feeling High Eₘₐₓ and a Good Effects Eₘₐₓ that are significantly reduced for the serdexmethylphenidate composition when compared to 40 mg d-methylphenidate hydrochloride following intranasal administration of the composition to a human or animal subject. In another embodiment, the serdexmethylphenidate is serdexmethylphenidate chloride and the amount of serdexmethylphenidate chloride is 80 mg per dose or less. In yet another embodiment, the serdexmethylphenidate is serdexmethylphenidate chloride and the amount of serdexmethylphenidate chloride is at least 80 mg per dose.

In some embodiments, the present technology provides a serdexmethylphenidate composition that provides a Feeling Drowsy/Alert Eₘₐₓ that is significantly reduced for the serdexmethylphenidate composition when compared to 40 mg d-methylphenidate hydrochloride following intranasal administration of the composition to a human or animal subject. In another embodiment, the serdexmethylphenidate is serdexmethylphenidate chloride and the amount of serdexmethylphenidate chloride is 80 mg per dose or less. In yet another embodiment, the serdexmethylphenidate is serdexmethylphenidate chloride and the amount of serdexmethylphenidate chloride is at least 80 mg per dose.

In some embodiments, the present technology provides a serdexmethylphenidate composition that provides an Any Effect Eₘₐₓ that is significantly reduced for the serdexmethylphenidate composition when compared to 40 mg d-methylphenidate hydrochloride following intranasal administration of the composition to a human or animal subject. In another embodiment, the serdexmethylphenidate is serdexmethylphenidate chloride and the amount of serdexmethylphenidate chloride is 80 mg per dose or less. In yet another embodiment, the serdexmethylphenidate is serdexmethylphenidate chloride and the amount of serdexmethylphenidate chloride is at least 80 mg per dose.

In some embodiments, the present technology provides a serdexmethylphenidate composition that provides an Ease of Nasal Insufflation Eₘₐₓ that is significantly increased for the serdexmethylphenidate composition when compared to 40 mg d-methylphenidate hydrochloride following intranasal administration of the composition to a human or animal subject. In another embodiment, the serdexmethylphenidate is serdexmethylphenidate chloride and the amount of serdexmethylphenidate chloride is 80 mg per dose or less. In yet another embodiment, the serdexmethylphenidate is serdexmethylphenidate chloride and the amount of serdexmethylphenidate chloride is at least 80 mg per dose.

In an alternative embodiment, the salt is a pharmaceutically acceptable salt.

In some embodiments, the present technology provides a least one method of intranasal administration of an amount of serdexmethylphenidate that results in abuse related effects that are lower compared to d-methylphenidate. In another embodiment, the serdexmethylphenidate is serdexmethylphenidate chloride and the d-methylphenidate is d-methylphenidate hydrochloride. In yet another embodiment, the amount of serdexmethylphenidate chloride is 80 mg per dose or less. In an alternative embodiment, the amount of serdexmethylphenidate chloride is at least 80 mg per dose. In yet another alternative embodiment, the abuse related effects are one or more of Drug Liking Eₘₐₓ, Feeling High Eₘₐₓ, Feeling Drowsy/Alert Eₘₐₓ, or Good Effects Eₘₐₓ.

In some embodiments, the present technology provides at least one method of intranasal administration of an amount of serdexmethylphenidate that results in abuse related effects that are not substantially different compared to a placebo. In another embodiment, the serdexmethylphenidate is serdexmethylphenidate chloride. In yet another embodiment, the amount of serdexmethylphenidate chloride is 80 mg per dose or less. In an alternative embodiment, the amount of serdexmethylphenidate chloride is at least 80 mg per dose. In yet another alternative embodiment, the abuse related effects are one or more of Drug Liking Eₘₐₓ, Feeling High Eₘₐₓ, Feeling Drowsy/Alert Eₘₐₓ, or Good Effects Eₘₐₓ.

In some embodiments, the present technology provides a composition comprising serdexmethylphenidate, or a salt thereof, wherein the composition has a dosage amount of serdexmethylphenidate that provides a mean Take Drug Again Eₘₐₓ that is not substantially different to placebo following intranasal administration of the composition to a human or animal subject. In another embodiment, the dosage amount is 80 mg or less. In yet another embodiment, the dosage amount is at least about 80 mg.

In some embodiments, the present technology provides a composition comprising serdexmethylphenidate, or a salt thereof, wherein the composition has a dosage amount of serdexmethylphenidate that provides a mean Overall Drug Liking Eₘₐₓ that is not substantially different to placebo following intranasal administration of the composition to a human or animal subject. In another embodiment, the dosage amount is 80 mg or less. In yet another embodiment, the dosage amount is at least about 80 mg.

In some embodiments, the present technology provides a composition comprising an amount of serdexmethylphenidate, or a salt thereof, that results in at least one improved abuse potential measure as compared to d-methylphenidate hydrochloride following intranasal administration of the composition to a human or animal subject. In another embodiment, the amount of serdexmethylphenidate, or a salt thereof, results in at least two improved abuse potential measures. In yet another embodiment, the amount of serdexmethylphenidate, or a salt thereof, results in at least three improved abuse potential measures. In yet a further embodiment, the amount of serdexmethylphenidate, or a salt thereof, results in at least four improved abuse potential measures. In an alternative embodiment, the improved abuse potential measure is a member selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

In some embodiments, the present technology provides a composition comprising an amount of serdexmethylphenidate, or a salt thereof, that results in at least one abuse potential measure that is not substantially different as compared to a placebo following intranasal administration of the composition to a human or animal subject. In another embodiment, the amount of serdexmethylphenidate, or a salt thereof, results in at least two abuse potential measures that are not substantially different as compared to a placebo. In yet another embodiment, the amount of serdexmethylphenidate, or a salt thereof, results in at least three abuse potential measures that are not substantially different as compared to a placebo. In a further embodiment, the amount of serdexmethylphenidate, or a salt thereof, results in at least four abuse potential measures that are not substantially different as compared to a placebo. In another alternative embodiment, the not substantially different abuse potential measure is a member selected from the group consisting of Take Drug Again Eₘₐₓ and Overall Drug Liking Eₘₐₓ.

In some embodiments, the present technology provides at least one method of intranasal administration of an amount of serdexmethylphenidate chloride, or a salt thereof, that results in at least one improved abuse potential measure as compared to d-methylphenidate hydrochloride. In another embodiment, the administration of serdexmethylphenidate, or a pharmaceutically acceptable salt thereof, results in at least two improved abuse potential measures. In yet another embodiment, the administration of serdexmethylphenidate, or a pharmaceutically acceptable salt thereof, results in at least three improved abuse potential measures. In a further embodiment, the administration of serdexmethylphenidate, or a pharmaceutically acceptable salt thereof, results in at least four improved abuse potential measures. In an alternative embodiment, the administration of serdexmethylphenidate, or a pharmaceutically acceptable salt thereof, results in at least five improved abuse potential measures. In yet a further alternative embodiment, the improved abuse potential member is selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

In some embodiments, the present technology provides at least one method of intranasal administration of an amount of serdexmethylphenidate chloride, or a salt thereof, that results in at least one abuse potential measure that is not substantially different as compared to placebo. In another embodiment, the administration of serdexmethylphenidate, or a pharmaceutically acceptable salt thereof, results in at least two abuse potential measures that are not substantially different as compared to a placebo. In yet another embodiment, the abuse potential measures comprise Take Drug Again Eₘₐₓ and/or Overall Drug Liking Eₘₐₓ.

In some embodiments, the present technology provides a composition comprising an amount of serdexmethylphenidate, or a pharmaceutically acceptable salt thereof, that results in at least one abuse potential measure that is not substantially different as compared to placebo following intranasal administration of the composition to a human or animal subject. In another embodiment, the composition that results in at least two abuse potential measures that are not substantially different as compared to placebo. In yet another embodiment, the abuse potential measures comprise Take Drug Again Eₘₐₓ and/or Overall Drug Liking Eₘₐₓ.

In some embodiments, the present technology provides a composition comprising serdexmethylphenidate, or a salt thereof, wherein when the composition exhibits a lower mean Drug Liking (“DL”) Eₘₐₓ when compared to d-methylphenidate following intravenous administration of the composition to a human or animal subject. In some embodiments, the composition exhibits a substantially lower mean Drug Liking Eₘₐₓ when compared to d-methylphenidate. In yet another embodiment, the composition comprises an amount of serdexmethylphenidate, or a salt thereof, per dose wherein the composition exhibits a substantially lower mean Drug Liking Eₘₐₓ when compared to 40 mg of d-methylphenidate hydrochloride per dose following intravenous administration of the composition to a human or animal subject. In yet another embodiment, the serdexmethylphenidate salt is serdexmethylphenidate chloride and the amount of serdexmethylphenidate chloride is 30 mg per dose or less. In an alternative embodiment, the serdexmethylphenidate salt is serdexmethylphenidate chloride and the amount of serdexmethylphenidate chloride is at least 30 mg per dose. In another embodiment, the composition exhibits a mean Drug Liking Eₘₐₓ that is substantially lower by a margin of at least 10 when compared to 15 mg of d-methylphenidate hydrochloride per dose.

In some embodiments, the present technology provides a serdexmethylphenidate chloride composition that provides statistically significant reductions in maximal Drug Liking Eₘₐₓ at 30 mg of serdexmethylphenidate chloride when compared to 15 mg d-methylphenidate hydrochloride following intravenous administration of the composition to a human or animal subject.

In some embodiments, the present technology provides a serdexmethylphenidate composition that provides retrospective endpoints of Take Drug Again Eₘₐₓ and Overall Drug Liking Eₘₐₓ that are significantly lower for the serdexmethylphenidate composition when compared to 15 mg d-methylphenidate hydrochloride following intravenous administration of the composition to a human or animal subject. In another embodiment, the serdexmethylphenidate is serdexmethylphenidate chloride and the amount of serdexmethylphenidate chloride is 30 mg per dose or less. In yet another embodiment, the serdexmethylphenidate is serdexmethylphenidate chloride and the amount of serdexmethylphenidate chloride is at least 30 mg per dose.

In some embodiments, the present technology provides a serdexmethylphenidate composition that provides a Feeling High Eₘₐₓ and a Good Effects Eₘₐₓ that are significantly reduced for the serdexmethylphenidate composition when compared to 15 mg d-methylphenidate hydrochloride following intravenous administration of the composition to a human or animal subject. In another embodiment, the serdexmethylphenidate is serdexmethylphenidate chloride and the amount of serdexmethylphenidate chloride is 30 mg per dose or less. In yet another embodiment, the serdexmethylphenidate is serdexmethylphenidate chloride and the amount of serdexmethylphenidate chloride is at least 30 mg per dose.

In some embodiments, the present technology provides a serdexmethylphenidate composition that provides a Feeling Drowsy/Alert Eₘₐₓ that is significantly reduced for the serdexmethylphenidate composition when compared to 15 mg d-methylphenidate hydrochloride following intravenous administration of the composition to a human or animal subject. In another embodiment, the serdexmethylphenidate is serdexmethylphenidate chloride and the amount of serdexmethylphenidate chloride is 30 mg per dose or less. In yet another embodiment, the serdexmethylphenidate is serdexmethylphenidate chloride and the amount of serdexmethylphenidate chloride is at least 30 mg per dose.

In some embodiments, the present technology provides a serdexmethylphenidate composition that provides an Any Effect Eₘₐₓ that is significantly reduced for the serdexmethylphenidate composition when compared to 15 mg d-methylphenidate hydrochloride following intravenous administration of the composition to a human or animal subject. In another embodiment, the serdexmethylphenidate is serdexmethylphenidate chloride and the amount of serdexmethylphenidate chloride is 30 mg per dose or less. In yet another embodiment, the serdexmethylphenidate is serdexmethylphenidate chloride and the amount of serdexmethylphenidate chloride is at least 30 mg per dose.

In an alternative embodiment, the salt is a pharmaceutically acceptable salt.

In some embodiments, the present technology provides a least one method of intravenous administration of an amount of serdexmethylphenidate that results in abuse related effects that are lower compared to d-methylphenidate. In another embodiment, the serdexmethylphenidate is serdexmethylphenidate chloride and the d-methylphenidate is d-methylphenidate hydrochloride. In yet another embodiment, the amount of serdexmethylphenidate chloride is 30 mg per dose or less. In an alternative embodiment, the amount of serdexmethylphenidate chloride is at least 30 mg per dose. In yet another alternative embodiment, the abuse related effects are one or more of Drug Liking Eₘₐₓ, Take Drug Again Eₘₐₓ, Feeling High Eₘₐₓ, Feeling Drowsy/Alert Eₘₐₓ, or Good Effects Eₘₐₓ.

In some embodiments, the present technology provides at least one method of intravenous administration of an amount of serdexmethylphenidate that results in abuse related effects that are substantially similar compared to a placebo. In another embodiment, the serdexmethylphenidate is serdexmethylphenidate chloride. In yet another embodiment, the amount of serdexmethylphenidate chloride is 30 mg per dose or less. In an alternative embodiment, the amount of serdexmethylphenidate chloride is at least 30 mg per dose. In yet another alternative embodiment, the abuse related effects are one or more of Drug Liking Eₘₐₓ, Feeling High Eₘₐₓ, Feeling Drowsy/Alert Eₘₐₓ, or Good Effects Eₘₐₓ.

In some embodiments, the present technology provides a composition comprising serdexmethylphenidate, or a salt thereof, wherein the composition has a dosage amount of serdexmethylphenidate that provides a mean Take Drug Again Eₘₐₓ that is not substantially different to placebo following intravenous administration of the composition to a human or animal subject. In another embodiment, the dosage amount is 30 mg or less. In yet another embodiment, the dosage amount is at least about 30 mg.

In some embodiments, the present technology provides a composition comprising serdexmethylphenidate, or a salt thereof, wherein the composition has a dosage amount of serdexmethylphenidate that provides a mean Overall Drug Liking Eₘₐₓ that is substantially similar to placebo following intravenous administration of the composition to a human or animal subject. In another embodiment, the dosage amount is 30 mg or less. In yet another embodiment, the dosage amount is at least about 30 mg.

In some embodiments, the present technology provides a composition comprising an amount of serdexmethylphenidate, or a salt thereof, that results in at least one improved abuse potential measure as compared to d-methylphenidate hydrochloride following intravenous administration of the composition to a human or animal subject. In another embodiment, the amount of serdexmethylphenidate, or a salt thereof, results in at least two improved abuse potential measures. In yet another embodiment, the amount of serdexmethylphenidate, or a salt thereof, results in at least three improved abuse potential measures. In yet a further embodiment, the amount of serdexmethylphenidate, or a salt thereof, results in at least four improved abuse potential measures. In an alternative embodiment, the improved abuse potential measure is a member selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

In some embodiments, the present technology provides a composition comprising an amount of serdexmethylphenidate, or a salt thereof, that results in at least one abuse potential measure that is substantially similar as compared to a placebo following intravenous administration of the composition to a human or animal subject. In another embodiment, the amount of serdexmethylphenidate, or a salt thereof, results in at least two abuse potential measures that are substantially similar as compared to a placebo. In yet another embodiment, the amount of serdexmethylphenidate, or a salt thereof, results in at least three abuse potential measures that are substantially similar as compared to a placebo. In a further embodiment, the amount of serdexmethylphenidate, or a salt thereof, results in at least four abuse potential measures that are substantially similar as compared to a placebo. In another alternative embodiment, the substantially similar abuse potential measure is a member selected from the group consisting of Drug Liking Eₘₐₓ, Overall Drug Liking Eₘₐₓ, Feeling High Eₘₐₓ, and Good Effects Eₘₐₓ.

In some embodiments, the present technology provides at least one method of intravenous administration of an amount of serdexmethylphenidate chloride, or a salt thereof, that results in at least one improved abuse potential measure as compared to d-methylphenidate hydrochloride. In another embodiment, the administration of serdexmethylphenidate, or a pharmaceutically acceptable salt thereof, results in at least two improved abuse potential measures. In yet another embodiment, the administration of serdexmethylphenidate, or a pharmaceutically acceptable salt thereof, results in at least three improved abuse potential measures. In a further embodiment, the administration of serdexmethylphenidate, or a pharmaceutically acceptable salt thereof, results in at least four improved abuse potential measures. In an alternative embodiment, the administration of serdexmethylphenidate, or a pharmaceutically acceptable salt thereof, results in at least five improved abuse potential measures. In yet a further alternative embodiment, the improved abuse potential member is selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

In some embodiments, the present technology provides at least one method of intravenous administration of an amount of serdexmethylphenidate chloride, or a salt thereof, that results in at least one abuse potential measure that is not substantially different as compared to placebo. In yet another embodiment, the abuse potential measures comprise Take Drug Again Eₘₐₓ.

In some embodiments, the present technology provides a composition comprising an amount of serdexmethylphenidate, or a pharmaceutically acceptable salt thereof, that results in at least one abuse potential measure that is substantially similar as compared to placebo following intravenous administration of the composition to a human or animal subject. In another embodiment, the composition that results in at least two abuse potential measures that are substantially similar as compared to placebo. In yet another embodiment, the administration of serdexmethylphenidate, or a pharmaceutically acceptable salt thereof, results in at least three abuse potential measures that are substantially similar as compared to placebo. In a further embodiment, the administration of serdexmethylphenidate, or a pharmaceutically acceptable salt thereof, results in at least four abuse potential measures that are substantially similar as compared to placebo. In yet another embodiment, the abuse potential measures comprise Drug Liking Eₘₐₓ, Overall Drug Liking Eₘₐₓ, Feeling High Eₘₐₓ, and Good Effects Eₘₐₓ.

Further aspects and embodiments of the present technology are described in the following paragraphs.

In at least further embodiments of the present technology, there is provided at least one method for attenuating or reducing one or more adverse effects associated with administration of a composition comprising methylphenidate to a human or animal subject in need thereof, comprising replacing at least a portion of the methylphenidate to be administered with a composition comprising a serdexmethylphenidate compound having the following chemical formula:

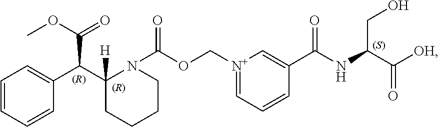

or salt of said compound, or mixtures thereof, and administering the composition comprising the serdexmethylphenidate compound to the human or animal subject, wherein administration of said composition attenuates or reduces adverse effects in said human or animal subject as compared to the adverse effects in a human subject or animal subject undergoing treatment with a composition consisting only of methylphenidate. The method of this aspect, wherein the salt is a pharmaceutically acceptable salt. The method of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,1-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The method of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The method of this aspect, wherein the pharmaceutically acceptable salt of the serdexmethylphenidate compound has the following structure:

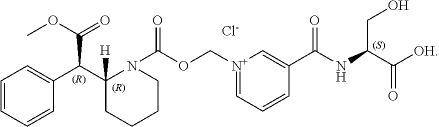

The method of this aspect, wherein the methylphenidate is methylphenidate is d-threo-methylphenidate, 1-threo-methylphenidate, d-erythro-methylphenidate, l-erythro-methylphenidate, salts thereof, or mixtures thereof. The method of this aspect, wherein the one or more adverse effects is selected from the group consisting of eye disorders or conditions, gastrointestinal disorders or conditions, nervous system disorders or conditions, psychiatric disorders or conditions, skin and subcutaneous disorders or conditions, vascular disorders or conditions, increased heartbeat, increased heart rate, increased blood pressure, chest pain, fever, joint pain, skin rash, or hives, nausea, headache, vomiting, decreased appetite, xerostomia, anxiety, tics, hyperhidrosis, euphoria, feeling high, dysphoria, irritability, palpitations, tachycardia, sinus tachycardia, abdominal discomfort, dry mouth, asthenia, feeling abnormal, feeling cold, feeling hot, feeling jittery, feeling of relaxation, dizziness, paraesthesia, somnolence, tremor, and/or combinations thereof. The method of this aspect, wherein the administration is selected from the group consisting of oral and transdermal administration. The method of this aspect, wherein the administration is oral administration. The method of this aspect, wherein the composition is administered in a dosage form selected from the group consisting of a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The method of this aspect, wherein oral administration of the composition results in reduced adverse effects when compared with a molar equivalent amount of unconjugated d-methylphenidate.

A method of treating or preventing disorder or condition symptoms in a human subject comprising administering to the subject a composition comprising a serdexmethylphenidate compound having the following chemical formula:

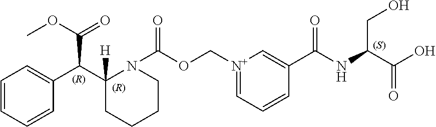

or salt of said compound, or mixtures thereof, wherein, following administration of the composition, at least one of the Cₘₐₓ, AUCₗₐₛₜ, or AUC_{inf} of d-methylphenidate active released from the composition administered to the human or animal subject is proportional across at least about a 1.5-fold dose range, preferably at least about a 2-fold dose range, preferably at least about a 5-fold dose range, preferably at least about a 10-fold dose range, preferably at least about a 15-fold dose range, preferably at least about a 50-fold dose range, preferably at least about a 100-fold dose range. The method of this aspect, wherein the salt is a pharmaceutically acceptable salt. The method of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The method of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The method of this aspect, wherein the pharmaceutically acceptable salt of the serdexmethylphenidate compound has the following structure:

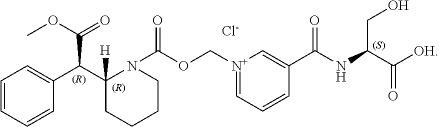

The method this aspect, wherein the disorder or condition is selected from the group consisting of attention deficit disorder (ADD, technically ADHD Predominantly Inattentive Type), attention-deficit hyperactivity disorder (ADHD), ADHD with tics, ADHD with Tourette syndrome, adjunctive therapy in major depressive disorder, amphetamine use disorder, Asperger's disorder, autism, autistic spectrum disorder, binge eating disorder, bipolar disorder, chemotherapy-associated fatigue, chronic fatigue syndrome, cocaine dependence, cocaine use disorder, depression, eating disorder, excessive daytime sleepiness (EDS), excessive sleepiness associated with obstructive sleep apnea, excessive sleepiness associated with shift work disorder, idiopathic hypersomnia, insomnia, major depressive disorder narcolepsy, methamphetamine use disorder, multiple sclerosis-associated fatigue, narcolepsy with cataplexy, obesity, pervasive developmental disorder, rejection sensitive dysphoria, schizophrenia, sleep disorder, and stimulant dependence. The method of this aspect, wherein the composition is used in a method of treating or preventing attention deficit disorder (ADD, technically ADHD Predominantly Inattentive Type), attention-deficit hyperactivity disorder (ADHD), ADHD with tics, or ADHD with Tourette syndrome in a human or animal subject. The method of this aspect, wherein the composition is in a multiple dose form or a single dose form. The method of this aspect, wherein the composition is provided in a unit dose form, blister pack, roll, or bulk bottle. The method of this aspect, wherein the administration is selected from the group consisting of oral and transdermal administration. The method of this aspect, wherein the administration is oral administration. The method of this aspect, wherein the composition is administered in a dosage form selected from the group consisting of a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The method of this aspect, wherein the composition further comprises unconjugated methylphenidate, salts thereof, or mixtures thereof. The method of this aspect, wherein the unconjugated methylphenidate is d-threo-methylphenidate, 1-threo-methylphenidate, d-erythro-methylphenidate, 1-erythro-methylphenidate, salts thereof, or mixtures thereof.

In further embodiments there is provided at least one method of minimizing adverse effects in a human or animal subject undergoing treatment with a composition comprising unconjugated methylphenidate said method comprising the steps of a) replacing the treatment with unconjugated methylphenidate with a treatment comprising a therapeutically effective amount of a composition comprising a serdexmethylphenidate compound having the following chemical formula:

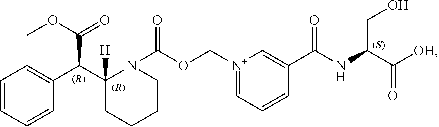

salt of the compound, or mixtures thereof, and b) administering said composition of serdexmethylphenidate compound to a human or animal subject in need thereof, wherein administration of said compound minimizes the adverse effects in said human or animal subject as compared to the adverse effects in a human or animal subject undergoing treatment with a composition consisting only of unconjugated methylphenidate. The method of this aspect, wherein the salt is a pharmaceutically acceptable salt. The method of this aspect, wherein the composition comprising the serdexmethylphenidate compound further comprises unconjugated methylphenidate, a salt thereof, or a mixture thereof. The method of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,1-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The method of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The method of this aspect, wherein the pharmaceutically acceptable salt of the serdexmethylphenidate compound has the following structure:

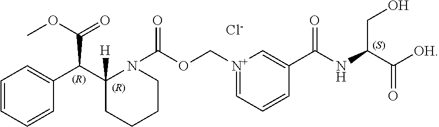

The method of this aspect, wherein the unconjugated methylphenidate is d-threo-methylphenidate, 1-threo-methylphenidate, d-erythro-methylphenidate, 1-erythro-methylphenidate, salts thereof, or mixtures thereof. The method of this aspect, wherein the salt of the unconjugated methylphenidate is hydrochloride.

A method of minimizing adverse effects in a human or animal subject undergoing treatment for ADHD, where the adverse effects results from administration of a composition comprising unconjugated methylphenidate, comprising the steps of selecting a human or animal subject undergoing treatment for ADHD, wherein said treatment comprises at least in part administration of a composition comprising unconjugated methylphenidate and replacing said treatment with a new treatment comprising a therapeutically effective amount of a composition that comprises a serdexmethylphenidate compound having the following chemical formula:

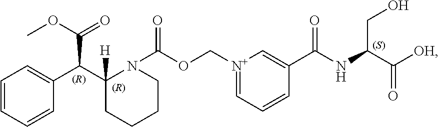

salt of said compound, or mixtures thereof. The method of this aspect, wherein the salt is a pharmaceutically acceptable salt. The method of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,1-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The method of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The method of this aspect, wherein the pharmaceutically acceptable salt of the serdexmethylphenidate compound has the following structure:

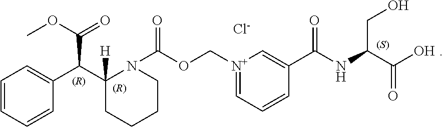

The method of this aspect, wherein the composition comprising the serdexmethylphenidate compound additionally comprises about 0% to about 10% by weight of unconjugated methylphenidate, preferably about 0% to about 5% by weight of unconjugated methylphenidate, preferably about 0% to about 2% by weight of unconjugated methylphenidate based on the total combined weight of methylphenidate active contained in the unconjugated methylphenidate and the serdexmethylphenidate composition. The method of this aspect, wherein the unconjugated methylphenidate is d-threo-methylphenidate, 1-threo-methylphenidate, d-erythro-methylphenidate, 1-erythro-methylphenidate, salts thereof, or mixtures thereof.

In some embodiments of the present technology, there is provided one or more methods of treating a human or animal subject having at least one disorder or condition requiring stimulation of the central nervous system of the human or animal subject, comprising administering to the human or animal subject a pharmaceutically effective amount of a composition comprising a serdexmethylphenidate compound having the following chemical formula:

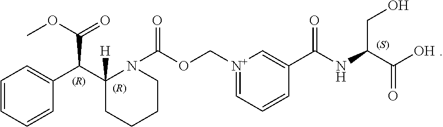

salt of the compound, or mixtures thereof, wherein the administration treats at least one disorder or condition requiring stimulation of the central nervous system of the human or animal subject, and wherein at least one of the Cₘₐₓ, AUCₗₐₛₜ, and/or AUC_{inf} of d-methylphenidate active released from the composition administered to the human or animal subject is proportional across at least about a 1.5-fold dose range, preferably at least about a 2-fold dose range, preferably at least about a 5-fold dose range, preferably at least about a 10-fold dose range, preferably at least about a 15-fold dose range, preferably at least about a 50-fold dose range, or preferably at least about a 100-fold dose range. The method of this aspect, wherein the salt is a pharmaceutically acceptable salt. The method of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,1-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The method of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The method of this aspect, wherein the pharmaceutically acceptable salt of the serdexmethylphenidate compound has the following structure:

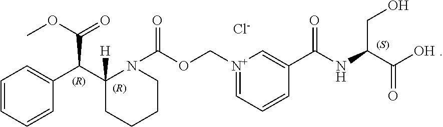

The method this aspect, wherein the disorder or condition is selected from the group consisting of attention deficit disorder (ADD, technically ADHD Predominantly Inattentive Type), attention-deficit hyperactivity disorder (ADHD), ADHD with tics, ADHD with Tourette syndrome, adjunctive therapy in major depressive disorder, amphetamine use disorder, Asperger's disorder, autism, autistic spectrum disorder, binge eating disorder, bipolar disorder, chemotherapy-associated fatigue, chronic fatigue syndrome, cocaine dependence, cocaine use disorder, depression, eating disorder, excessive daytime sleepiness (EDS), excessive sleepiness associated with obstructive sleep apnea, excessive sleepiness associated with shift work disorder, idiopathic hypersomnia, insomnia, major depressive disorder narcolepsy, methamphetamine use disorder, multiple sclerosis-associated fatigue, narcolepsy with cataplexy, obesity, pervasive developmental disorder, rejection sensitive dysphoria, schizophrenia, sleep disorder, and stimulant dependence. The method of this aspect wherein the composition is used in a method of treating or preventing attention deficit disorder (ADD, technically ADHD Predominantly Inattentive Type), attention-deficit hyperactivity disorder (ADHD), ADHD with tics, or ADHD with Tourette syndrome in a human or animal subject. The method of this aspect, wherein the administration is selected from the group consisting of oral or transdermal administration. The method of this aspect, wherein the administration is oral administration. The method of this aspect, wherein the wherein the composition is in a dosage form selected from the group consisting of a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The method of this aspect, wherein the serdexmethylphenidate in the composition is co-formulated with unconjugated methylphenidate. The method of this aspect, wherein the unconjugated methylphenidate is d-threo-methylphenidate, 1-threo-methylphenidate, d-erythro-methylphenidate, 1-erythro-methylphenidate, salts thereof, or mixtures thereof. The method of this aspect, wherein the serdexmethylphenidate compound is present in the composition in an amount that is the molar equivalent to a dose of d-methylphenidate in the range of about 0.1 mg to about 1100 mg per dose, preferably in the range of about 0.1 to about 500 mg per dose, preferably in the range of about 500 mg to about 1100 mg per dose\, preferably in the range of about 200 mg to about 1100 mg per dose, preferably in the range of about 300 mg to about 1050 mg per dose, preferably in the range of about 400 mg to about 1000 mg per dose, preferably in the range of about 500 mg to about 1000 mg per dose, preferably in the range of about 0.5 mg to about 480 mg per dose, preferably in the range of about 1 mg to about 250 mg per dose, preferably in the range of about 2 mg to about 240 mg per dose, preferably in the range of about 5 mg to about 200 mg per dose, preferably in the range of about 10 mg to about 150 mg per dose, preferably in the range of about 20 mg to about 100 mg per dose, preferably in the range of about 30 mg to about 80 mg per dose, or preferably in the range of about 40 mg to about 70 mg per dose. The method of this aspect, wherein the serdexmethylphenidate compound is present in the composition in an amount that is molar equivalent to a dose of d-methylphenidate in the range of about 500 mg to about 1100 mg per dose. The method of this aspect, wherein the composition has a dose mixture of about 1 mg to about 20 mg d-methylphenidate hydrochloride and about 20 mg to about 160 mg serdexmethylphenidate chloride, preferably about 6 mg d-methylphenidate hydrochloride and about 28 mg serdexmethylphenidate chloride, preferably about 9 mg d-methylphenidate hydrochloride and about 42 mg serdexmethylphenidate chloride, preferably about 8 mg d-methylphenidate hydrochloride and about 64 mg serdexmethylphenidate chloride, preferably about 12 mg d-methylphenidate hydrochloride and about 56 mg serdexmethylphenidate chloride, or preferably about 16 mg d-methylphenidate hydrochloride and about 48 mg serdexmethylphenidate chloride. The method of this aspect, wherein daily administration of the composition provides a steady-state plasma concentration of released d-methylphenidate after about 24 hours of once-a-day dosing administration, preferably after about 48 hours of once-a-day dosing administration, preferably after about 72 hours of once-a-day dosing administration, preferably after about 96 hours of once-a-day dosing administration, or preferably after about 120 hours of once-a-day dosing administration.

The method of this aspect, wherein the unconjugated methylphenidate contributes a molar dose amount in the range of about 5% to about 95%, preferably in the range of about 10% to about 90%, preferably in the range of about 20% to about 80%, preferably in the range of about 25% to about 75%, preferably in the range of about 30% to about 70%, preferably in the range of about 40% to about 60%, preferably in the range of about 50%; and the serdexmethylphenidate compound contributes a molar dose amount in the range of about 95% to about 5%, preferably in the range of about 90% to about 10%, preferably in the range of about 80% to about 20%, preferably in the range of about 75% to about 25%, preferably in the range of about 70% to about 30%, preferably in the range of about 60% to about 40%, or preferably in the range of about 50%, based on the total combined molar dose of the unconjugated d-methylphenidate and the serdexmethylphenidate compound. The method of this aspect, wherein the total molar dose in the composition comprises about 90% serdexmethylphenidate and about 10% unconjugated methylphenidate, preferably about 80% serdexmethylphenidate and about 20% unconjugated methylphenidate, preferably about 75% serdexmethylphenidate and about 25% unconjugated methylphenidate, preferably about 70% serdexmethylphenidate and about 30% unconjugated methylphenidate, preferably about 60% serdexmethylphenidate and about 40% unconjugated methylphenidate, preferably about 50% serdexmethylphenidate and about 50% methylphenidate. The method this aspect, wherein the total molar dose of the composition comprises about 90% serdexmethylphenidate and about 10% unconjugated methylphenidate or about 70% serdexmethylphenidate and about 30% unconjugated methylphenidate. The method of this aspect, wherein the unconjugated methylphenidate is d-threo-methylphenidate, 1-threo-methylphenidate, d-erythro-methylphenidate, 1-erythro-methylphenidate, salts thereof, or mixtures thereof. The method this aspect, wherein the composition has a dosing regimen of at least once a week, preferably every other day, preferably one time a day, preferably about two times a day, preferably about three times a day, preferably about four times a day or more. The method of this aspect, wherein the composition has a dosing regimen of at least once one time a day. The method of this aspect, wherein the composition has a dosage strength of serdexmethylphenidate, or a total combined dosage strength of unconjugated methylphenidate and serdexmethylphenidate that is the molar equivalent to an individual dose in the range of about 0.1 mg to about 1100 mg per dose, preferably in the range of about 0.1 to about 500 mg per dose, preferably in the range of about 500 mg to about 1100 mg per dose, preferably in the range of about 200 mg to about 1100 mg per dose, preferably in the range of about 300 mg to about 1050 mg per dose, preferably in the range of about 400 mg to about 1000 mg per dose, preferably in the range of about 500 mg to about 1000 mg per dose, preferably in the range of about 0.5 mg to about 480 mg per dose, preferably in the range of about 1 mg to about 250 mg per dose, preferably in the range of about 2 mg to about 240 mg per dose, preferably in the range of about 5 mg to about 200 mg per dose, preferably in the range of about 10 mg to about 150 mg per dose, preferably in the range of about 20 mg to about 100 mg per dose, preferably in the range of about 30 mg to about 80 mg per dose, or preferably in the range of about 40 mg to about 70 mg per dose. The method of this aspect, wherein the serdexmethylphenidate compound is present in the composition in an amount that is molar equivalent to a dose of d-methylphenidate in the range of about 500 mg to about 1100 mg per dose. The method of this aspect, wherein the composition has a dose mixture of about 1 mg to about 20 mg d-methylphenidate hydrochloride and about 20 mg to about 160 mg serdexmethylphenidate chloride, preferably about 6 mg d-methylphenidate hydrochloride and about 28 mg serdexmethylphenidate chloride, preferably about 9 mg d-methylphenidate hydrochloride and about 42 mg serdexmethylphenidate chloride, preferably about 8 mg d-methylphenidate hydrochloride and about 64 mg serdexmethylphenidate chloride, preferably about 12 mg d-methylphenidate hydrochloride and about 56 mg serdexmethylphenidate chloride, or preferably about 16 mg d-methylphenidate hydrochloride and about 48 mg serdexmethylphenidate chloride. The method of this aspect, the composition comprises a pharmaceutically acceptable salt of serdexmethylphenidate and a pharmaceutically acceptable salt of unconjugated methylphenidate. The method of this aspect, wherein the human subject is a selected from the group consisting of a pediatric subject, a normative subject, an adult subject, and an adolescent subject. Alternatively, wherein the method of this aspect is for a human subject that can be an elderly subject.

A pharmaceutical kit comprising: at least two sets of doses in a package, each set having an amount of individual doses in the set, wherein each individual dose in a first set comprises a composition comprising unconjugated methylphenidate, salt thereof, or mixtures thereof, and each individual dose in a second set comprises a composition comprising serdexmethylphenidate, salt thereof, or mixtures thereof, and instructions for use. The pharmaceutical kit of this aspect, wherein the combined dose of at least two individual doses of the first set and the second set are therapeutically effective. The pharmaceutical kit of this aspect, wherein the salt is a pharmaceutically acceptable salt. The pharmaceutical kit of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,1-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The pharmaceutical kit of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The pharmaceutical kit of this aspect, wherein the pharmaceutically acceptable salt of the serdexmethylphenidate compound has the following structure:

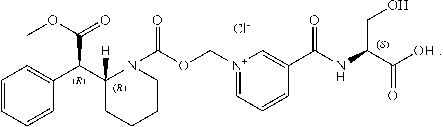

The pharmaceutical kit of this aspect, wherein the instructions provide a method or treating a disorder or condition selected from the group consisting of attention deficit disorder (ADD, technically ADHD Predominantly Inattentive Type), attention-deficit hyperactivity disorder (ADHD), ADHD with tics, ADHD with Tourette syndrome, adjunctive therapy in major depressive disorder, amphetamine use disorder, Asperger's disorder, autism, autistic spectrum disorder, binge eating disorder, bipolar disorder, chemotherapy-associated fatigue, chronic fatigue syndrome, cocaine dependence, cocaine use disorder, depression, eating disorder, excessive daytime sleepiness (EDS), excessive sleepiness associated with obstructive sleep apnea, excessive sleepiness associated with shift work disorder, idiopathic hypersomnia, insomnia, major depressive disorder narcolepsy, methamphetamine use disorder, multiple sclerosis-associated fatigue, narcolepsy with cataplexy, obesity, pervasive developmental disorder, rejection sensitive dysphoria, schizophrenia, sleep disorder, and stimulant dependence. The pharmaceutical kit of this aspect, wherein the composition is used in a method of treating or preventing attention deficit disorder (ADD, technically ADHD Predominantly Inattentive Type), attention-deficit hyperactivity disorder (ADHD), ADHD with tics, or ADHD with Tourette syndrome in a human or animal subject. The pharmaceutical kit of this aspect, wherein the human subject is a selected from the group consisting of a pediatric subject, a normative subject, an adult subject, and an adolescent subject. The pharmaceutical kit of this aspect, wherein the human subject can also be an elderly subject. The pharmaceutical kit of this aspect, wherein the instructions for use comprise instructions for combining at least one dose from the first and second set with at least one dose in the second set into a single dose. The pharmaceutical kit of this aspect, wherein the doses are provided in a unit dose form, blister pack, roll, or bulk bottle. The pharmaceutical kit of this aspect, wherein the individual doses have a dosing regimen of at least once a week, preferably every other day, preferably one time a day, preferably about two times a day, preferably about three times a day, preferably about four times a day or more. The pharmaceutical kit of this aspect, wherein the individual doses have a dosing regimen of one time a day. The pharmaceutical kit of this aspect, wherein the kit comprises from about 1 to about 100 individual doses. The pharmaceutical kit of this aspect, wherein the kit comprises from about 10 to about 30 individual doses. The pharmaceutical kit of this aspect, wherein the kit comprises from about 1 to about 7 individual doses. The pharmaceutical kit of this aspect, wherein the kit comprises from about 1 to about 14 individual doses. The pharmaceutical kit of this aspect, wherein the kit comprises from about 1 to about 21 individual doses. The pharmaceutical kit of this aspect, wherein the composition further comprises one or more excipients or one or more additional pharmaceutically active ingredients. The pharmaceutical kit of this aspect, wherein the excipients are selected from the group consisting of anti-adherents, antioxidants, binders, coatings, disintegrants, gel forming agents, fillers, flavors, colors, colorants, glidants, lubricants, preservatives, sorbents and sweeteners.

The present technology also provides in at least some embodiments, at least one method of intranasal administration of an amount of serdexmethylphenidate that results in at least one of the following: abuse related effects that are lower or at least one improved abuse potential measure as compared to intranasal administration of the same active or molar amount of unconjugated d-methylphenidate. The method of this aspect, wherein the intranasal administration of an amount of serdexmethylphenidate reduces or prevents at least one adverse effect related to unconjugated methylphenidate. The method of this aspect, wherein the serdexmethylphenidate is serdexmethylphenidate chloride and the unconjugated d-methylphenidate is d-methylphenidate hydrochloride. The method of this aspect, wherein the amount of serdexmethylphenidate chloride is about 80 mg per dose or less. The method of this aspect, wherein the amount of serdexmethylphenidate chloride is at least 80 mg per dose. The method of this aspect, wherein the abuse related effects are one or more of Drug Liking Eₘₐₓ, Feeling High Eₘₐₓ, Feeling Drowsy/Alert Eₘₐₓ, or Good Effects Eₘₐₓ. The method of this aspect, wherein the administration of serdexmethylphenidate, or a pharmaceutically acceptable salt thereof, results in at least two improved abuse potential measures, preferably at least three improved abuse potential measures, preferably at least four improved abuse potential measures, or preferably at least five improved abuse potential measures. The method of this aspect, wherein the improved abuse potential measure is selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ. The method of this aspect, wherein the salt is a pharmaceutically acceptable salt. The method of this aspect, wherein the pharmaceutically acceptable salt is independently selected from the group consisting of acetate, 1-aspartate, besylate, bicarbonate, carbonate, d-camsylate, 1-camsylate, citrate, edisylate, formate, fumarate, gluconate, hydrobromide/bromide, hydrochloride/chloride, d-lactate, 1-lactate, d,1-lactate, d,1-malate, 1-malate, mesylate, pamoate, phosphate, succinate, sulfate, bisulfate, d-tartrate, martrate, d,1-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, undecylenate, and combinations thereof. The method of this aspect, wherein the pharmaceutically acceptable salt is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite, sodium, potassium, calcium, magnesium, lithium, cholinate, lysinium, ammonium, and combinations thereof. The method of this aspect, wherein the pharmaceutically acceptable salt of the serdexmethylphenidate compound has the following structure:

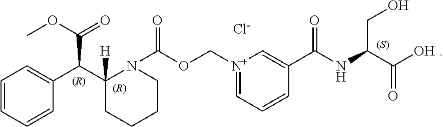

A composition comprising: a serdexmethylphenidate conjugate having the following chemical formula:

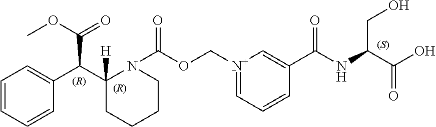

wherein, following administration of the composition, each of the Cₘₐₓ, AUCₗₐₛₜ, and AUC_{inf} of d-methylphenidate active from the composition is dose-proportional across at least about a 1.5-fold dose range. The composition of this aspect wherein each of the Cₘₐₓ, AUCₗₐₛₜ, and AUC_{inf} is dose-proportional across at least about a 2-fold dose range. The composition of this aspect wherein each of the Cₘₐₓ, AUCₗₐₛₜ, and AUC_{inf} is dose-proportional across at least about a 5-fold dose range. The composition of this aspect wherein each of the Cₘₐₓ, AUCₗₐₛₜ, and AUC_{inf} is dose-proportional across at least about a 10-fold dose range. The composition of this aspect wherein each of the AUCₗₐₛₜ and AUC_{inf} is dose-proportional across at least about a 15-fold dose range. The composition of this aspect wherein AUC_{inf} is dose-proportional across at least about a 25-fold dose range. The composition of this aspect wherein AUC_{inf} is dose-proportional across at least about a 50-fold dose range. The composition of this aspect wherein AUC_{inf} is dose-proportional across at least about a 100-fold dose range. The composition of this aspect, wherein the serdexmethylphenidate conjugate is present in the composition in an amount that is molar equivalent to a dose of d-methylphenidate in the range of about 0.1 mg to about 500 mg per day. The composition of this aspect wherein the serdexmethylphenidate conjugate is present in the composition in an amount that is molar equivalent to a dose of d-methylphenidate in the range of about 0.5 mg to about 480 mg per day. The composition of this aspect wherein the serdexmethylphenidate conjugate is present in the composition in an amount that is molar equivalent to a dose of d-methylphenidate in the range of about 1 mg to about 250 mg per day. The composition of this aspect wherein the serdexmethylphenidate conjugate is present in the composition in an amount that is molar equivalent to a dose of d-methylphenidate in the range of about 2 mg to about 240 mg per day. The composition of this aspect wherein the serdexmethylphenidate conjugate is present in the composition in an amount that is molar equivalent to a dose of d-methylphenidate in the range of about 5 mg to about 200 mg per day. The composition of this aspect wherein the serdexmethylphenidate conjugate is present in the composition in an amount that is molar equivalent to a dose of d-methylphenidate in the range of about 10 mg to about 150 mg per day. The composition this aspect wherein the serdexmethylphenidate conjugate is present in the composition in an amount that is molar equivalent to a dose of d-methylphenidate in the range of about 20 mg to about 100 mg per day. The composition of this aspect the serdexmethylphenidate conjugate is present in the composition in an amount that is molar equivalent to a dose of d-methylphenidate in the range of about 30 mg to about 80 mg per day. The composition of this aspect wherein the serdexmethylphenidate conjugate is present in the composition in an amount that is molar equivalent to a dose of d-methylphenidate in the range of about 40 mg to about 70 mg per day. The composition of this aspect, wherein the administration is selected from the group consisting of oral, intranasal, and transdermal administration. The composition of this aspect wherein the composition is in a dosage form selected from the group consisting of a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The composition of this aspect wherein Cₘₐₓ, AUCₗₐₛₜ, and AUC_{inf} of d-methylphenidate active from the composition is dose-proportional across about a 6-fold, about a 11-fold, or about a 82-fold dose range, respectively. The composition of this aspect, wherein the serdexmethylphenidate conjugate exhibits an improved AUC and rate of release over time when compared to unconjugated d-methylphenidate over the same time period; exhibits less variability in the PK profile when compared to unconjugated d-methylphenidate; or has reduced adverse effects when compared with unconjugated d-methylphenidate. The composition of this aspect, wherein the serdexmethylphenidate conjugate is provided in an amount sufficient to provide a therapeutically effective amount of d-methylphenidate. The composition of this aspect, wherein the serdexmethylphenidate conjugate is provided in an amount sufficient to provide a therapeutically equivalent AUC and Cₘₐₓ when compared to an equivalent molar amount of unconjugated d-methylphenidate. The composition of this aspect, wherein the serdexmethylphenidate conjugate is provided in an amount sufficient to provide a therapeutically equivalent AUC and a lower Cₘₐₓ when compared to an equivalent molar amount of unconjugated d-methylphenidate. The composition of this aspect, wherein the serdexmethylphenidate conjugate is provided in an amount sufficient to provide a therapeutic effect but provides a lower AUC and a lower Cₘₐₓ when compared to an equivalent molar amount of unconjugated d-methylphenidate. The composition of this aspect wherein the unconjugated d-methylphenidate comprises d-methylphenidate.

A composition comprising: (a) unconjugated d-methylphenidate, wherein the unconjugated d-methylphenidate comprises d-methylphenidate, and (b) a serdexmethylphenidate conjugate having the following chemical formula:

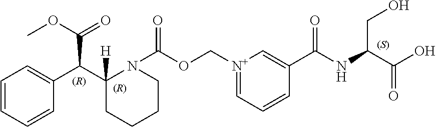

wherein the unconjugated d-methylphenidate and the serdexmethylphenidate conjugate is present in the composition in an amount that is molar equivalent to a dose of d-methylphenidate in the range of about 0.1 mg to about 300 mg, and wherein, following administration of the composition, each of the Cₘₐₓ, AUCₗₐₛₜ, and AUC_{inf} of d-methylphenidate active from the composition is dose-proportional across at least a 1.5-fold dose range. The composition of this aspect wherein each of the Cₘₐₓ, AUCₗₐₛₜ, and AUC_{inf} is dose-proportional across at least a 2-fold dose range. The composition of this aspect wherein each of the Cₘₐₓ, AUCₗₐₛₜ, and AUC_{inf} is dose-proportional across at least a 5-fold dose range. The composition of this aspect wherein each of the Cₘₐₓ, AUCₗₐₛₜ, and AUC_{inf} is dose-proportional across at least a 10-fold dose range. The composition of this aspect wherein each of the AUCₗₐₛₜ and AUC_{inf} is dose-proportional across at least a 15-fold dose range. The composition of this aspect wherein AUC_{inf} is dose-proportional across at least a 25-fold dose range. The composition of this aspect wherein AUC_{inf} is dose-proportional across at least a 50-fold dose range. The composition of this aspect wherein AUC_{inf} is dose-proportional across at least a 100-fold dose range. The composition of this aspect, wherein administration results in minimized and/or reduced adverse effects in terms of severity, frequency, and/or duration as compared to compositions comprising unconjugated d-methylphenidate administered at equimolar doses. The composition of this aspect, wherein the serdexmethylphenidate conjugate is present in the composition in an amount that is molar equivalent to a dose of d-methylphenidate in the range of about 0.1 mg to about 500 mg per day. The composition of this aspect wherein the serdexmethylphenidate conjugate is present in the composition in an amount that is molar equivalent to a dose of d-methylphenidate in the range of about 0.5 mg to about 480 mg per day. The composition of this aspect wherein the serdexmethylphenidate conjugate is present in the composition in an amount that is molar equivalent to a dose of d-methylphenidate in the range of about 1 mg to about 250 mg per day. The composition of this aspect wherein the serdexmethylphenidate conjugate is present in the composition in an amount that is molar equivalent to a dose of d-methylphenidate in the range of about 2 mg to about 240 mg per day. The composition of this aspect wherein the serdexmethylphenidate conjugate is present in the composition in an amount that is molar equivalent to a dose of d-methylphenidate in the range of about 5 mg to about 200 mg per day. The composition of this aspect wherein the serdexmethylphenidate conjugate is present in the composition in an amount that is molar equivalent to a dose of d-methylphenidate in the range of about 10 mg to about 150 mg per day. The composition of this aspect wherein the serdexmethylphenidate conjugate is present in the composition in an amount that is molar equivalent to a dose of d-methylphenidate in the range of about 20 mg to about 100 mg per day. The composition of this aspect wherein the serdexmethylphenidate conjugate is present in the composition in an amount that is molar equivalent to a dose of d-methylphenidate in the range of about 30 mg to about 80 mg per day. The composition of this aspect wherein the serdexmethylphenidate conjugate is present in the composition in an amount that is molar equivalent to a dose of d-methylphenidate in the range of about 40 mg to about 70 mg per day. The composition of this aspect, wherein the administration is selected from the group consisting of oral, intranasal, and transdermal administration. The composition of this aspect, wherein the composition is in a dosage form selected from the group consisting of: a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The composition of this aspect wherein Cₘₐₓ, AUCₗₐₛₜ, and AUC_{inf} of d-methylphenidate active from the composition is dose-proportional across a 6-fold, 11-fold, and 82-fold dose range, respectively. The composition of this aspect, wherein the unconjugated d-methylphenidate contributes a molar dose amount in the range of about 5% to about 95% and the serdexmethylphenidate conjugate contributes a molar dose amount in the range of about 95% to about 5%, based on the total combined molar dose of the unconjugated d-methylphenidate and the serdexmethylphenidate conjugate. The composition of this aspect, wherein the unconjugated d-methylphenidate contributes a molar dose amount in the range of about 10% to about 90% and the serdexmethylphenidate conjugate contributes a molar dose amount in the range of about 90% to about 10%, based on the total combined molar dose of the unconjugated d-methylphenidate and the serdexmethylphenidate conjugate. The composition of this aspect, wherein the unconjugated d-methylphenidate contributes a molar dose amount in the range of about 20% to about 80% and the serdexmethylphenidate conjugate contributes a molar dose amount in the range of about 80% to about 20%, based on the total combined molar dose of the unconjugated d-methylphenidate and the serdexmethylphenidate conjugate. The composition of this aspect, wherein the unconjugated d-methylphenidate contributes a molar dose amount in the range of about 25% to about 75% and the serdexmethylphenidate conjugate contributes a molar dose amount in the range of about 75% to about 25%, based on the total combined molar dose of the unconjugated d-methylphenidate and the serdexmethylphenidate conjugate. The composition of this aspect, wherein the unconjugated d-methylphenidate contributes a molar dose amount in the range of about 30% to about 70% and the serdexmethylphenidate conjugate contributes a molar dose amount in the range of about 70% to about 30%, based on the total combined molar dose of the unconjugated d-methylphenidate and the serdexmethylphenidate conjugate. The composition of this aspect, wherein the unconjugated d-methylphenidate contributes a molar dose amount in the range of about 40% to about 60% and the serdexmethylphenidate conjugate contributes a molar dose amount in the range of about 60% to about 40%, based on the total combined molar dose of the unconjugated d-methylphenidate and the serdexmethylphenidate conjugate. The composition of this aspect, wherein the unconjugated d-methylphenidate contributes a molar dose amount of about 50% and the serdexmethylphenidate conjugate contributes a molar dose amount of about 50%, based on the total combined molar dose of the unconjugated d-methylphenidate and the serdexmethylphenidate conjugate. The composition of this aspect, wherein the composition has a dosing regimen of at least once a week. The composition of this aspect wherein the composition has a dosing regimen of every other day. The composition of this aspect, wherein the dosing regimen is used in a method for the treatment of binge eating disorder. The composition of this aspect, wherein the composition has a dosing regimen of one time a day. The composition of this aspect, wherein the composition has a dosing regimen of about two times a day. The composition of this aspect, wherein the composition has a dosing regimen of about three times a day. The composition of this aspect, wherein the composition has a dosing regimen of about four times a day or more. The composition of this aspect, wherein the composition has a dosage strength of serdexmethylphenidate, or a total combined dosage strength of unconjugated d-methylphenidate and serdexmethylphenidate that is the molar equivalent to an individual dose of about 1 mg to about 100 mg d-methylphenidate. The composition of this aspect, wherein the total molar dose in the composition comprises about 90% serdexmethylphenidate and about 10% unconjugated d-methylphenidate. The composition of this aspect, wherein the total molar dose in the composition comprises about 80% serdexmethylphenidate and about 20% unconjugated d-methylphenidate. The composition of this aspect, wherein the total molar dose in the composition comprises about 75% serdexmethylphenidate and about 25% unconjugated d-methylphenidate. The composition of this aspect, wherein the total molar dose in the composition comprises about 70% serdexmethylphenidate and about 30% unconjugated d-methylphenidate. The composition of this aspect, wherein the total molar dose in the composition comprises about 60% serdexmethylphenidate and about 40% unconjugated d-methylphenidate. The composition of this aspect, wherein the total molar dose in the composition comprises about 50% serdexmethylphenidate and about 50% d-methylphenidate. The composition of this aspect, wherein the composition comprises a salt of d-methylphenidate and a salt of serdexmethylphenidate. The composition of this aspect, wherein the composition has a dose mixture of about 1 mg to about 20 mg d-methylphenidate hydrochloride and about 20 mg to about 80 mg serdexmethylphenidate chloride. The composition of this aspect, wherein the dose mixture is about 6 mg d-methylphenidate hydrochloride and about 28 mg serdexmethylphenidate chloride. The composition of this aspect, wherein the dose mixture is about 9 mg d-methylphenidate hydrochloride and about 42 mg serdexmethylphenidate chloride. The composition of this aspect, wherein the dose mixture is about 8 mg d-methylphenidate hydrochloride and about 64 mg serdexmethylphenidate chloride. The composition of this aspect, wherein the dose mixture is about 12 mg d-methylphenidate hydrochloride and about 56 mg serdexmethylphenidate chloride. The composition of this aspect, wherein the dose mixture is about 16 mg d-methylphenidate hydrochloride and about 48 mg serdexmethylphenidate chloride.

A composition comprising a serdexmethylphenidate conjugate having the following chemical formula:

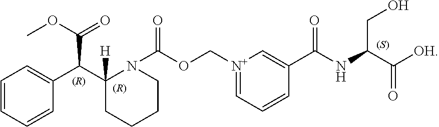

or a pharmaceutically acceptable salt thereof, wherein the composition results in minimized and/or reduced adverse effects in terms of severity, frequency, and/or duration after administration to a human or animal subject when compared to an equivalent molar amount of administered unconjugated d-methylphenidate. The composition of this aspect, wherein the pharmaceutically acceptable salt the of serdexmethylphenidate conjugate is serdexmethylphenidate chloride. The composition of this aspect wherein the composition further comprises unconjugated d-methylphenidate, wherein the unconjugated d-methylphenidate comprises a pharmaceutically acceptable salt of d-methylphenidate. The composition of this aspect, where in the pharmaceutically acceptable salt of d-methylphenidate is d-methylphenidate hydrochloride. The composition of this aspect, wherein the composition provides a lower AUC and/or Cₘₐₓ for d-methylphenidate released from the serdexmethylphenidate conjugate when compared to an equivalent molar amount of unconjugated d-methylphenidate following intravenous or intranasal administration of the composition to a human or animal subject. The composition of this aspect, wherein the lower AUC is about 10% to about 15% of the AUC for the unconjugated d-methylphenidate after intravenous administration to a human or animal subject. The composition of this aspect, wherein the lower Cₘₐₓ is about 20% of the Cₘₐₓ for unconjugated d-methylphenidate after intravenous administration to a human or animal subject. The composition of this aspect, wherein the composition provides a lower Take Drug Again score at 12 and 24 hours post-dose administration when compared to an equivalent molar amount of the unconjugated d-methylphenidate following intravenous administration of the composition to a human or animal subject. The composition of this aspect, wherein the composition provides a lower maximum (Eₘₐₓ) Feeling High score when compared to an equivalent molar amount of the unconjugated d-methylphenidate following intravenous administration of the composition to a human or animal subject. The composition of this aspect, wherein the composition provides a lower maximum (Eₘₐₓ) Good Effects score when compared to an equivalent molar amount of unconjugated d-methylphenidate following intravenous administration of the composition to a human or animal subject. The composition of this aspect, wherein the composition provides a Take Drug Again scores at 12 and 24 hours post-dose administration that is not substantially different when compared to a placebo following intravenous administration of the composition to a human or animal subject. The composition of this aspect, wherein the composition provides a maximum (Eₘₐₓ) Feeling High score that is substantially similar when compared to a placebo following intravenous administration of the composition to a human or animal subject. The composition of this aspect, wherein the composition provides a lower Overall Drug Liking scores at 12 and 24 hours post-dose administration when compared to an equivalent molar amount of unconjugated d-methylphenidate following intravenous administration of the composition to a human or animal subject. The composition of this aspect, wherein the composition provides an Overall Drug Liking scores at 12 and 24 hours post-dose administration that is substantially similar when compared to a placebo following intravenous administration of the composition to a human or animal subject. The composition of this aspect, wherein the composition provides a maximal (Eₘₐₓ) Feeling High score that is substantially similar when compared to a placebo following intravenous administration of the composition to a human or animal subject. The composition of this aspect, wherein the composition provides a maximal (Eₘₐₓ) Good Effects score that is substantially similar when compared to a placebo following intravenous administration of the composition to a human or animal subject. The composition of this aspect, wherein there is a substantial difference in the median maximum (Eₘₐₓ) Drug Liking score when the composition is compared to an equivalent molar amount of unconjugated d-methylphenidate following intravenous administration to a human or animal subject. The composition of this aspect, wherein the median maximum (Eₘₐₓ) Drug Liking score is substantially similar when the composition is compared to a placebo following intravenous administration to a human or animal subject. The composition of this aspect, wherein there is a substantial difference in the median maximum (Eₘₐₓ) Overall Drug Liking score and the median Overall Drug Liking scores at 12 and 24 hours post-dose administration when the composition is compared to an equivalent molar amount of unconjugated d-methylphenidate following intravenous administration to a human or animal subject. The composition of this aspect, wherein the median maximum (Eₘₐₓ) Overall Drug Liking score and the median Overall Drug Liking scores at 12 and 24 hours post-dose administration are substantially similar when the composition is compared to a placebo following intravenous administration to a human or animal subject. The composition of this aspect, wherein there is a substantial difference in the mean Take Drug Again scores at 12 and 24 hours post-dose administration when the composition is compared to an equivalent molar amount of unconjugated d-methylphenidate following intravenous administration to a human or animal subject. The composition of this aspect, wherein the mean Take Drug Again scores at 12 and 24 hours post-dose administration are not substantially different when the composition is compared to a placebo following intravenous administration to a human or animal subject. The composition of this aspect, wherein there is a substantial difference in the median maximal (Eₘₐₓ) Feeling High score when the composition is compared to an equivalent molar amount of unconjugated d-methylphenidate following intravenous administration to a human or animal subject. The composition of this aspect, wherein the mean maximal (Eₘₐₓ) Feeling High score is substantially similar when the composition is compared to a placebo following intravenous administration to a human or animal subject. The composition of this aspect, wherein there is a substantial difference in the median maximal (Eₘₐₓ) Good Effects score when the composition is compared to an equivalent molar amount of unconjugated d-methylphenidate following intravenous administration to a human or animal subject. The composition of this aspect, wherein the mean maximal (Eₘₐₓ) Good Effects score is substantially similar when the composition is compared to a placebo following intravenous administration to a human or animal subject. The composition of this aspect, wherein the human subject is a member selected from the group consisting of a pediatric subject, an elderly subject, a normative subject, a neonatal subject, and an adolescent subject. The composition of this aspect, wherein the administration is selected from the group consisting of oral, intravenous, intranasal, and transdermal administration. The composition of this aspect, wherein the composition is in a dosage form selected from the group consisting of: a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The composition of this aspect, wherein the one or more adverse effects is selected from the group consisting of cardiac disorders, eye disorders, gastrointestinal disorders, nervous system disorders, psychiatric disorders, skin and subcutaneous disorders, and vascular disorders. The composition of this aspect, wherein the adverse effects are selected from the group consisting of increased heartbeat, increased blood pressure, chest pain, fever, joint pain, skin rash, or hives, nausea, headache, vomiting, decreased appetite, xerostomia, anxiety, tics, hyperhidrosis, euphoria, and irritability.

A method for attenuating or reducing one or more adverse effects associated with administration of a composition comprising unconjugated d-methylphenidate to a human or animal subject in need thereof, comprising replacing at least part of the unconjugated d-methylphenidate to be administered with a composition comprising serdexmethylphenidate, and administering the composition comprising serdexmethylphenidate to the human or animal subject. The method of this aspect, wherein the one or more adverse effects is selected from the group consisting of cardiac disorders, eye disorders, gastrointestinal disorders, nervous system disorders, psychiatric disorders, skin and subcutaneous disorders, and vascular disorders. The method of this aspect, wherein the adverse effects are selected from the group consisting of increased heartbeat, increased blood pressure, chest pain, fever, joint pain, skin rash, or hives, nausea, headache, vomiting, decreased appetite, xerostomia, anxiety, tics, hyperhidrosis, euphoria, and irritability. The method of this aspect, wherein the administration is selected from the group consisting of oral, intravenous, intranasal, and transdermal administration. The method of this aspect, wherein the composition is administered in a dosage form selected from the group consisting of: a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The method of this aspect, wherein oral administration of the composition results in reduced adverse effects when compared with a molar equivalent amount of unconjugated d-methylphenidate. The method of this aspect, wherein the human subject is a selected from the group consisting of a pediatric subject, an elderly subject, a normative subject, a neonatal subject, and an adolescent subject.

In, still further embodiments of the present technology, there is provided one or more methods of treating or preventing attention deficit hyperactivity disorder symptoms in a human subject comprising administering to the subject a composition comprising serdexmethylphenidate, wherein, following administration of the composition, the human or animal subject has a Cₘₐₓ, AUCₗₐₛₜ, and AUC_{inf} of d-methylphenidate active from the composition administered to the human or animal subject that is proportional across at least a 1.5-fold dose range. The method of this aspect wherein each of the Cₘₐₓ, AUCₗₐₛₜ, and AUC_{inf} is dose-proportional across at least a 2-fold dose range. The method of this aspect wherein each of the Cₘₐₓ, AUCₗₐₛₜ, and AUC_{inf} is dose-proportional across at least a 5-fold dose range. The method of this aspect wherein each of the Cₘₐₓ, AUCₗₐₛₜ, and AUC_{inf} is dose-proportional across at least a 10-fold dose range. The method of this aspect wherein each of the AUCₗₐₛₜ and AUC_{inf} is dose-proportional across at least a 15-fold dose range. The method of this aspect wherein AUC_{inf} is dose-proportional across at least a 25-fold dose range. The method of this aspect wherein AUC_{inf} is dose-proportional across at least a 50-fold dose range. The method of this aspect wherein AUC_{inf} is dose-proportional across at least a 100-fold dose range. The method of this aspect, wherein the composition is in a single dose form. The method of this aspect, wherein the composition is in a multiple dose form. The method of this aspect, wherein the human subject is a selected from the group consisting of a pediatric subject, an elderly subject, a normative subject, a neonatal subject, and an adolescent subject.

A method of minimizing adverse effects in a human or animal subject undergoing treatment with a composition comprising unconjugated d-methylphenidate said method comprising the steps of a) replacing at least some of the composition comprising unconjugated d-methylphenidate with a therapeutically equivalent amount of a composition comprising serdexmethylphenidate and b) administering said composition of unconjugated d-methylphenidate and serdexmethylphenidate to a human or animal subject in need thereof. The method of this aspect, wherein the human subject is a selected from the group consisting of a pediatric subject, an elderly subject, a normative subject, a neonatal subject, and an adolescent subject.

A method of minimizing adverse effects in a human or animal subject undergoing treatment for ADHD, where the adverse effects results from administration of a composition comprising unconjugated d-methylphenidate, comprising the steps of selecting a human or animal subject undergoing treatment for ADHD and administering to said human or animal subject a composition that replaces the unconjugated d-methylphenidate with a therapeutically equivalent composition comprising serdexmethylphenidate. The method of this aspect, wherein the composition comprising serdexmethylphenidate additionally comprises 0 to about 10% by weight of unconjugated d-methylphenidate, based on the total combined weight of d-methylphenidate active contained in the unconjugated d-methylphenidate and the serdexmethylphenidate conjugate. The method of this aspect wherein the human subject is a selected from the group consisting of a pediatric subject, an elderly subject, a normative subject, a neonatal subject, and an adolescent subject.

A method of treating a human or animal subject having at least one disorder or condition requiring stimulation of the central nervous system of the human or animal subject, comprising administering to the human or animal subject a pharmaceutically effective amount of a composition comprising serdexmethylphenidate, wherein the administration treats at least one disorder or condition requiring stimulation of the central nervous system of the human or animal subject, and wherein the Cₘₐₓ, AUCₗₐₛₜ, and AUC_{inf} of d-methylphenidate active from the composition administered to the human or animal subject are proportional across at least a 1.5-fold dose range. The method of this aspect wherein each of the Cₘₐₓ, AUCₗₐₛₜ, and AUC_{inf} is dose-proportional across at least a 2-fold dose range. The method of this aspect wherein each of the Cₘₐₓ, AUCₗₐₛₜ, and AUC_{inf} is dose-proportional across at least a 5-fold dose range. The method of this aspect wherein each of the Cₘₐₓ, AUCₗₐₛₜ, and AUC_{inf} is dose-proportional across at least a 10-fold dose range. The method of this aspect wherein each of the AUCₗₐₛₜ and AUC_{inf} is dose-proportional across at least a 15-fold dose range. The method of this aspect wherein AUC_{inf} is dose-proportional across at least a 25-fold dose range. The method of this aspect wherein AUC_{inf} is dose-proportional across at least a 50-fold dose range. The method of this aspect wherein AUC_{inf} is dose-proportional across at least a 100-fold dose range. The method of this aspect wherein the administration is selected from the group consisting of oral, intravenous, intranasal, and transdermal administration. The method of this aspect, wherein the wherein the composition is in a dosage form selected from the group consisting of: a tablet, a capsule, a caplet, a gel, a suppository, a troche, a lozenge, an oral powder, a solution, an oral film, a thin strip, a slurry, a soft gel capsule, a syrup, an orally disintegrating tablet, a chewable tablet, and a suspension. The method of this aspect, wherein the serdexmethylphenidate in the composition is co-formulated with unconjugated d-methylphenidate. The method of this aspect, wherein the serdexmethylphenidate conjugate is present in the composition in an amount that is the molar equivalent to a dose of d-methylphenidate in the range of about 0.1 to about 500 mg per day. The method of this aspect wherein the serdexmethylphenidate conjugate is present in the composition in an amount that is molar equivalent to a dose of d-methylphenidate in the range of about 0.5 mg to about 480 mg per day. The method of this aspect wherein the serdexmethylphenidate conjugate is present in the composition in an amount that is molar equivalent to a dose of d-methylphenidate in the range of about 1 mg to about 250 mg per day. The method of this aspect wherein the serdexmethylphenidate conjugate is present in the composition in an amount that is molar equivalent to a dose of d-methylphenidate in the range of about 2 mg to about 240 mg per day. The method of this aspect wherein the serdexmethylphenidate conjugate is present in the composition in an amount that is molar equivalent to a dose of d-methylphenidate in the range of about 5 mg to about 200 mg per day. The method of this aspect wherein the serdexmethylphenidate conjugate is present in the composition in an amount that is molar equivalent to a dose of d-methylphenidate in the range of about 10 mg to about 150 mg per day. The method of this aspect wherein the serdexmethylphenidate conjugate is present in the composition in an amount that is molar equivalent to a dose of d-methylphenidate in the range of about 20 mg to about 100 mg per day. The method of this aspect wherein the serdexmethylphenidate conjugate is present in the composition in an amount that is molar equivalent to a dose of d-methylphenidate in the range of about 30 mg to about 80 mg per day. The method of this aspect wherein the serdexmethylphenidate conjugate is present in the composition in an amount that is molar equivalent to a dose of d-methylphenidate in the range of about 40 mg to about 70 mg per day. The method of this aspect, wherein daily administration of the composition provides a steady-state plasma concentration of released d-methylphenidate after about 24 hours of once-a-day dosing administration. The method of this aspect, wherein daily administration of the composition provides a steady-state plasma concentration of released d-methylphenidate after about 48 hours of once-a-day dosing administration. The method of this aspect, wherein daily administration of the composition provides a steady-state plasma concentration of released d-methylphenidate after about 72 hours of once-a-day dosing administration. The method of this aspect, wherein daily administration of the composition provides a steady-state plasma concentration of released d-methylphenidate after about 96 hours of once-a-day dosing administration. The method of this aspect, wherein daily administration of the composition provides a steady-state plasma concentration of released d-methylphenidate after about 120 hours of once-a-day dosing administration. The method of this aspect, wherein the unconjugated d-methylphenidate contributes a dose amount in the range of about 5% to about 95% and the serdexmethylphenidate contributes a dose amount in the range of about 95% to about 5%, based on the total combined molar dose of the unconjugated d-methylphenidate and the serdexmethylphenidate conjugate. The method of this aspect, wherein the unconjugated d-methylphenidate contributes a molar dose amount in the range of about 10% to about 90% and the serdexmethylphenidate contributes a molar dose amount in the range of about 90% to about 10%, based on the total combined molar dose of the unconjugated d-methylphenidate and the serdexmethylphenidate conjugate. The method of this aspect, wherein the unconjugated d-methylphenidate contributes a molar dose amount in the range of about 20% to about 80% and the serdexmethylphenidate contributes a molar dose amount in the range of about 80% to about 20%, based on the total combined molar dose of the unconjugated d-methylphenidate and the serdexmethylphenidate conjugate. The method of this aspect, wherein the unconjugated d-methylphenidate contributes a molar dose amount in the range of about 25% to about 75% and the serdexmethylphenidate contributes a molar dose amount in the range of about 75% to about 25%, based on the total combined molar dose of the unconjugated d-methylphenidate and the serdexmethylphenidate conjugate. The method of this aspect, wherein the unconjugated d-methylphenidate contributes a molar dose amount in the range of about 30% to about 70% and the serdexmethylphenidate contributes a molar dose amount in the range of about 70% to about 30%, based on the total combined molar dose of the unconjugated d-methylphenidate and the serdexmethylphenidate conjugate. The method of this aspect, wherein the unconjugated d-methylphenidate contributes a molar dose amount in the range of about 40% to about 60% and the serdexmethylphenidate contributes a molar dose amount in the range of about 60% to about 40%, based on the total combined molar dose of the unconjugated d-methylphenidate and the serdexmethylphenidate conjugate. The method of this aspect, wherein the unconjugated d-methylphenidate contributes a molar dose amount of about 50% and the serdexmethylphenidate contributes a molar dose amount of about 50%, based on the total combined molar dose of the unconjugated d-methylphenidate and the serdexmethylphenidate conjugate. The method of this aspect, wherein the composition has a dosing regimen of at least once a week. The method of this aspect, wherein the composition has a dosing regimen of every other day. The method of this aspect, wherein the dosing regimen is used in a method for the treatment of binge eating disorder. The method of this aspect, wherein the composition has a dosing regimen of one time a day. The method of this aspect, wherein the composition has a dosing regimen of about two times a day. The method of this aspect, wherein the composition has a dosing regimen of about three times a day. The method of this aspect, wherein the composition has a dosing regimen of about four times a day or more. The method of this aspect, wherein the composition has a dosage strength of serdexmethylphenidate, or a total combined dosage strength of unconjugated d-methylphenidate and serdexmethylphenidate that is the molar equivalent to an individual dose of about 1 mg to about 100 mg d-methylphenidate. The method of this aspect, wherein the total molar dose in the composition comprises about 90% serdexmethylphenidate and about 10% unconjugated d-methylphenidate. The method of this aspect, wherein the total molar dose in the composition comprises about 80% serdexmethylphenidate and about 20% unconjugated d-methylphenidate. The method of this aspect, wherein the total molar dose in the composition comprises about 75% serdexmethylphenidate and about 25% unconjugated d-methylphenidate. The method of this aspect, wherein the total molar dose in the composition comprises about 70% serdexmethylphenidate and about 30% unconjugated d-methylphenidate. The method of this aspect, wherein the total molar dose in the composition comprises about 60% serdexmethylphenidate and about 40% unconjugated d-methylphenidate. The method of this aspect, wherein the total molar dose in the composition comprises about 50% serdexmethylphenidate and about 50% d-methylphenidate. The method of this aspect, wherein the composition comprises a pharmaceutically acceptable salt of serdexmethylphenidate and a pharmaceutically acceptable salt of d-methylphenidate. The method of this aspect, wherein the composition has a dose mixture of about 1 mg to about 20 mg d-methylphenidate hydrochloride and about 20 mg to about 160 mg serdexmethylphenidate chloride. The method of this aspect, wherein the dose mixture is about 6 mg d-methylphenidate hydrochloride and about 28 mg serdexmethylphenidate chloride. The method of this aspect, wherein the dose mixture is about 9 mg d-methylphenidate hydrochloride and about 42 mg serdexmethylphenidate chloride. The method of this aspect, wherein the dose mixture is about 8 mg d-methylphenidate hydrochloride and about 64 mg serdexmethylphenidate chloride. The method of this aspect, wherein the dose mixture is about 12 mg d-methylphenidate hydrochloride and about 56 mg serdexmethylphenidate chloride. The method of this aspect, wherein the dose mixture is about 16 mg d-methylphenidate hydrochloride and about 48 mg serdexmethylphenidate chloride. The method of this aspect, wherein the human subject is a selected from the group consisting of a pediatric subject, an elderly subject, a normative subject, a neonatal subject, and an adolescent subject.

In some further embodiments, and/or aspects, the present technology provides one or more pharmaceutical kits comprising: at least two sets of doses in a package, each set having a specified amount of individual doses in the set, wherein the at least two combined individual doses of the at least two sets of doses are therapeutically effective, each individual dose in one set comprises a composition comprising unconjugated d-methylphenidate, and each individual dose in a second set comprises a composition comprising serdexmethylphenidate, and instructions for use. The pharmaceutical kit of this aspect, wherein the instructions for use comprise a method of treating or preventing attention deficit hyperactivity disorder symptoms in a human or animal subject. The pharmaceutical kit of this aspect, wherein the instructions for use instruct that a dose from the first set and/or a dose from the second set be administered to a human or animal subject depending on the human or animal subject's dose response, tolerability and/or need of duration of effect. The pharmaceutical kit of this aspect, wherein the human subject is a selected from the group consisting of a pediatric subject, an elderly subject, a normative subject, a neonatal subject, and an adolescent subject.

A pharmaceutical composition for treating a disorder or condition requiring stimulation of the central nervous system comprising a serdexmethylphenidate conjugate having the following chemical formula:

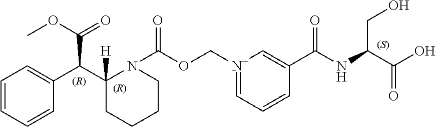

wherein administration results in minimized and/or reduced adverse effects in terms of severity, frequency, and/or duration as compared to compositions comprising unconjugated d-methylphenidate administered at equimolar doses. The pharmaceutical composition of this aspect, wherein the disorder or condition requiring the stimulation of the central nervous system is selected from the group consisting of ADD (technically ADHD Predominantly Inattentive Type), ADHD with tics, ADHD with Tourette syndrome, adjunctive therapy in major depressive disorder, amphetamine use disorder, Asperger's disorder, attention-deficit hyperactivity disorder (ADHD), autism, autistic spectrum disorder, binge eating disorder, bipolar disorder, chemotherapy-associated fatigue, chronic fatigue syndrome, cocaine dependence, cocaine use disorder, depression, eating disorder, excessive daytime sleepiness (EDS), excessive sleepiness associated with obstructive sleep apnea, excessive sleepiness associated with shift work disorder, idiopathic hypersomnia, insomnia, major depressive disorder narcolepsy, methamphetamine use disorder, multiple sclerosis-associated fatigue, narcolepsy with cataplexy, obesity, pervasive developmental disorder, schizophrenia, sleep disorder, and stimulant dependence.

A process for the preparation of serdexmethylphenidate conjugate polymorphs comprising the step of using crystallization conditions to isolate a free-base and salt forms and/or by ball-milling such forms.

Further aspects and embodiments of the present technology are described in the following paragraphs.

A composition comprising serdexmethylphenidate wherein the composition exhibits a lower mean Drug Liking (“DL”) Eₘₐₓ when compared to Focalin® XR following oral administration. The composition of this aspect, wherein the composition exhibits a statistically significant lower mean Drug Liking Eₘₐₓ when compared to Focalin® XR. The composition of this aspect, comprising an amount of serdexmethylphenidate, or a pharmaceutical salt thereof, per dose wherein the composition exhibits a statistically lower mean Drug Liking Eₘₐₓ when compared to 80 mg of Focalin® XR per dose. The composition of this aspect, wherein the serdexmethylphenidate is serdexmethylphenidate chloride, and the amount of serdexmethylphenidate chloride is 120 mg per dose or less. The composition of this aspect, wherein the serdexmethylphenidate is serdexmethylphenidate chloride, and the amount of serdexmethylphenidate chloride is at least 120 mg per dose. The composition of this aspect, wherein the serdexmethylphenidate is serdexmethylphenidate chloride, and the amount of serdexmethylphenidate chloride is 240 mg per dose or less. The composition of this aspect, wherein the serdexmethylphenidate is serdexmethylphenidate chloride, and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate wherein the composition exhibits a lower mean Drug Liking Eₘₐₓ when compared to phentermine hydrochloride following oral administration. The composition of this aspect, wherein the composition exhibits a statistically significantly lower mean Drug Liking Eₘₐₓ when compared to phentermine hydrochloride. The composition of this aspect, comprising an amount of serdexmethylphenidate, or a pharmaceutical salt thereof, per dose wherein the composition exhibits a statistically significantly lower mean Drug Liking Eₘₐₓ when compared to 60 mg of phentermine hydrochloride per dose. The composition of this aspect, wherein the serdexmethylphenidate is serdexmethylphenidate chloride, and the amount of serdexmethylphenidate chloride is 120 mg per dose or less. The composition of this aspect, wherein the serdexmethylphenidate is serdexmethylphenidate chloride, and the amount of serdexmethylphenidate chloride is at least 120 mg per dose. The composition of this aspect, wherein the serdexmethylphenidate is serdexmethylphenidate chloride, and the amount of serdexmethylphenidate chloride is 240 mg per dose or less. The composition of this aspect, wherein the serdexmethylphenidate is serdexmethylphenidate chloride, and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising up to 240 mg of serdexmethylphenidate, or a pharmaceutical salt thereof, wherein the composition exhibits a mean Drug Liking Eₘₐₓ that is lower than for a 60 mg dosage amount of phentermine hydrochloride following oral administration.

A composition comprising up to 120 mg of serdexmethylphenidate, or a pharmaceutical salt thereof, wherein the composition exhibits a mean Drug Liking Eₘₐₓ that is statistically significantly lower than for a 60 mg dosage amount of phentermine hydrochloride following oral administration. The composition of this aspect, wherein the composition exhibits a mean Drug Liking Eₘₐₓ that is statistically lower by a margin of at least 10 when compared to 80 mg of Focalin® XR per dose. The composition of this aspect, wherein the composition exhibits a mean Drug Liking Eₘₐₓ that is statistically lower by a margin of at least 10 when compared to 80 mg of Focalin® XR per dose. The composition of this aspect, wherein the composition exhibits a mean Drug Liking Eₘₐₓ that is statistically lower by a margin of at least 10 when compared to 60 mg of phentermine hydrochloride per dose. The composition of this aspect, wherein the composition exhibits a mean Drug Liking Eₘₐₓ that is statistically lower by a margin of at least 9 when compared to 60 mg of phentermine hydrochloride per dose.

A composition comprising serdexmethylphenidate wherein the composition exhibits a statistically similar mean Take Drug Again (“TDA”) Eₘₐₓ when compared to Focalin® XR following oral administration. The composition of this aspect, comprising an amount of serdexmethylphenidate, or a pharmaceutical salt thereof, per dose wherein the composition exhibits a statistically similar mean Take Drug Again Eₘₐₓ when compared to 80 mg of Focalin® XR per dose. The composition of this aspect, wherein the serdexmethylphenidate is serdexmethylphenidate chloride, and the amount of serdexmethylphenidate chloride is 120 mg per dose or less. The composition of this aspect, wherein the serdexmethylphenidate is serdexmethylphenidate chloride, and the amount of serdexmethylphenidate chloride is 240 mg per dose or less. The composition of this aspect, wherein the serdexmethylphenidate is serdexmethylphenidate chloride, and the amount of serdexmethylphenidate chloride is at least 120 mg per dose. The composition of this aspect, wherein the serdexmethylphenidate is serdexmethylphenidate chloride, and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate wherein the composition exhibits a lower mean Take Drug Again (“TDA”) Eₘₐₓ when compared to phentermine hydrochloride following oral administration. The composition of this aspect, wherein the composition exhibits a statistically significantly lower mean Take Drug Again Eₘₐₓ when compared to phentermine hydrochloride. The composition of this aspect, comprising an amount of serdexmethylphenidate, or a pharmaceutical salt thereof, per dose wherein the composition exhibits a statistically significantly lower mean Take Drug Again Eₘₐₓ when compared to 60 mg of phentermine hydrochloride per dose. The composition of this aspect, wherein the serdexmethylphenidate is serdexmethylphenidate chloride, and the amount of serdexmethylphenidate chloride is 120 mg per dose or less. The composition of this aspect, wherein the serdexmethylphenidate is serdexmethylphenidate chloride, and the amount of serdexmethylphenidate chloride is 240 mg per dose or less. The composition of this aspect, wherein the serdexmethylphenidate is serdexmethylphenidate chloride, and the amount of serdexmethylphenidate chloride is at least 120 mg per dose. The composition of this aspect, wherein the serdexmethylphenidate is serdexmethylphenidate chloride, and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate, comprising an amount of serdexmethylphenidate, or a pharmaceutical salt thereof, per dose wherein the composition exhibits a substantially similar mean Overall Drug Liking (“ODL”) Eₘₐₓ when compared to 80 mg of Focalin® XR per dose following oral administration. The composition of this aspect, wherein the serdexmethylphenidate is serdexmethylphenidate chloride, and the amount of serdexmethylphenidate chloride is 120 mg per dose or less. The composition of this aspect, wherein the serdexmethylphenidate is serdexmethylphenidate chloride, and the amount of serdexmethylphenidate chloride is 240 mg per dose or less. The composition of this aspect, wherein the serdexmethylphenidate is serdexmethylphenidate chloride, and the sufficient amount of serdexmethylphenidate chloride is at least 120 mg per dose. The composition of this aspect, wherein the serdexmethylphenidate is serdexmethylphenidate chloride, and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate, wherein the composition exhibits a lower mean Overall Drug Liking Eₘₐₓ when compared to phentermine hydrochloride following oral administration. The composition of this aspect, wherein the composition exhibits a statistically significantly lower mean Overall Drug Liking Eₘₐₓ when compared to phentermine hydrochloride. The composition of this aspect, comprising an amount of serdexmethylphenidate, or a pharmaceutical salt thereof, per dose wherein the composition exhibits a statistically significantly lower mean Overall Drug Liking Eₘₐₓ when compared to 60 mg of phentermine hydrochloride per dose. The composition of this aspect, wherein the serdexmethylphenidate is serdexmethylphenidate chloride, and the amount of serdexmethylphenidate chloride is 120 mg per dose or less. The composition of this aspect, wherein the serdexmethylphenidate is serdexmethylphenidate chloride, and the amount of serdexmethylphenidate chloride is 240 mg per dose or less. The composition of this aspect, wherein the serdexmethylphenidate is serdexmethylphenidate chloride, and the amount of serdexmethylphenidate chloride is at least 120 mg per dose. The composition of this aspect, wherein the serdexmethylphenidate is serdexmethylphenidate chloride, and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate wherein the composition exhibits a mean Drug Liking Eₘₐₓ that is significantly lower statistically than twice the maximum daily clinical dose of Focalin® XR following oral administration, wherein the maximum clinical daily dose is 40 mg.

A composition comprising serdexmethylphenidate wherein the compositions exhibits a mean Drug Liking Eₘₐₓ that is significantly lower statistically following an oral dose of 120 mg of serdexmethylphenidate chloride as compared to twice the maximum daily clinical dose of phentermine, wherein the maximum clinical daily dose is 30 mg.

A serdexmethylphenidate composition that provides statistically significant reductions in maximal Drug Liking (Eₘₐₓ) at 120 mg and 240 mg of serdexmethylphenidate chloride when compared to Focalin® XR (80 mg) and at 120 mg serdexmethylphenidate chloride when compared to phentermine (60 mg) following oral administration.

A serdexmethylphenidate composition that provides retrospective endpoints of Take Drug Again Eₘₐₓ and Overall Drug Liking Eₘₐₓ that are significantly lower for the serdexmethylphenidate composition versus phentermine at both 120 mg and 240 mg doses of serdexmethylphenidate following oral administration.

A serdexmethylphenidate composition that provides Feeling High Eₘₐₓ and Good Effects Eₘₐₓ that are significantly reduced for both 120 mg and 240 mg doses of serdexmethylphenidate when compared to Focalin® XR and phentermine following oral administration.

A method of orally administering serdexmethylphenidate chloride that results in abuse related effects that are lower compared to Focalin® XR. The method of this aspect, wherein the amount of serdexmethylphenidate chloride is up to 240 mg. The method of this aspect, wherein the abuse related effects are one or more of Drug Liking Eₘₐₓ, Feeling High Eₘₐₓ, Bad Effects Eₘₐₓ, or Good Effects Eₘₐₓ.

A method of orally administering an amount of serdexmethylphenidate chloride that results in abuse related effects that are lower compared to phentermine. The method of this aspect, wherein the amount of serdexmethylphenidate chloride is about 120 mg to about 240 mg. The method of this aspect, wherein the abuse related effects are one or more of Take Drug Again Eₘₐₓ Overall Drug Liking Eₘₐₓ, Feeling High Eₘₐₓ, Bad Effects Eₘₐₓ, or Good Effects Eₘₐₓ.

A method of orally administering an amount of serdexmethylphenidate chloride that results in a Drug Liking Eₘₐₓ that is statistically lower than phentermine. The method of this aspect, wherein the amount of serdexmethylphenidate chloride is about 120 mg to about 240 mg.

A composition comprising serdexmethylphenidate, or a pharmaceutical salt thereof, wherein the composition has a dosage amount of serdexmethylphenidate chloride that provides a Take Drug Again Eₘₐₓ that is statistically similar to placebo following oral administration. The composition of this aspect, wherein the dosage amount is 120 mg or less. The composition of this aspect, wherein the dosage amount is 240 mg or less. The composition of this aspect, wherein the dosage amount is at least about 120 mg. The composition of this aspect, wherein the dosage amount is at least about 240 mg. The composition of this aspect, wherein the dosage amount is about 120 mg to at least about 240 mg.

A composition comprising serdexmethylphenidate, or a pharmaceutical salt thereof, wherein the composition has a dosage amount of serdexmethylphenidate chloride that provides an Overall Drug Liking Eₘₐₓ that is statistically similar to placebo following oral administration. The composition of this aspect, wherein the dosage amount is 120 mg or less. The composition of this aspect, wherein the dosage amount is 240 mg or less. The composition of this aspect, wherein the dosage amount is at least about 120 mg. The composition of this aspect, wherein the dosage amount is at least about 240 mg. The composition of this aspect, wherein the dosage amount is about 120 mg to at least about 240 mg.

Further aspects and embodiments of the present technology are described in the following paragraphs.

A composition comprising serdexmethylphenidate wherein the composition exhibits a lower mean Drug Liking Eₘₐₓ when compared to Focalin® XR following oral administration. The composition of this aspect, wherein the composition exhibits a substantially lower mean Drug Liking Eₘₐₓ when compared to Focalin® XR. The composition of this aspect, comprising an amount of serdexmethylphenidate, or a pharmaceutical salt thereof, per dose wherein the composition exhibits a substantially lower mean Drug Liking Eₘₐₓ when compared to 80 mg of Focalin® XR per dose. The composition of this aspect, wherein the serdexmethylphenidate is serdexmethylphenidate chloride and the amount of serdexmethylphenidate chloride is 120 mg per dose or less. The composition of this aspect, wherein the serdexmethylphenidate is serdexmethylphenidate chloride and the amount of serdexmethylphenidate chloride is at least 120 mg per dose. The composition of this aspect, wherein the serdexmethylphenidate is serdexmethylphenidate chloride and the amount of serdexmethylphenidate chloride is 240 mg per dose or less. The composition of this aspect, wherein the serdexmethylphenidate is serdexmethylphenidate chloride and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate wherein the composition exhibits a lower mean Drug Liking Eₘₐₓ when compared to phentermine following oral administration. The composition of this aspect, wherein the composition exhibits a substantially lower mean Drug Liking Eₘₐₓ when compared to phentermine. The composition of this aspect ,comprising an amount of serdexmethylphenidate, or a pharmaceutical salt thereof, per dose wherein the composition exhibits a substantially lower mean Drug Liking Eₘₐₓ when compared to 60 mg of phentermine hydrochloride per dose. The composition of this aspect, wherein the serdexmethylphenidate is serdexmethylphenidate chloride and the amount of serdexmethylphenidate chloride is 120 mg per dose or less. The composition of this aspect, wherein the serdexmethylphenidate is serdexmethylphenidate chloride and the amount of serdexmethylphenidate chloride is at least 120 mg per dose. The composition of this aspect, wherein the serdexmethylphenidate is serdexmethylphenidate chloride and the amount of serdexmethylphenidate chloride is 240 mg per dose or less. The composition of this aspect, wherein the serdexmethylphenidate is serdexmethylphenidate chloride and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising up to 240 milligrams of serdexmethylphenidate chloride wherein the composition exhibits a mean Drug Liking Eₘₐₓ that is lower than for a 60 mg dosage amount of phentermine hydrochloride following oral administration.

A composition comprising up to 120 milligrams of serdexmethylphenidate chloride wherein the composition exhibits a mean Drug Liking Eₘₐₓ that is substantially lower than for a 60 mg dosage amount of phentermine hydrochloride following oral administration. The composition of this aspect, wherein the composition exhibits a mean Drug Liking Eₘₐₓ that is substantially lower by a margin of at least 10 when compared to 80 mg of Focalin® XR per dose. The composition of this aspect, wherein the composition exhibits a mean Drug Liking Eₘₐₓ that is substantially lower by a margin of at least 10 when compared to 80 mg of Focalin® XR per dose. The composition of this aspect, wherein the composition exhibits a mean Drug Liking Eₘₐₓ that is substantially lower by a margin of at least 10 when compared to 60 mg of phentermine hydrochloride per dose. The composition of this aspect, wherein the composition exhibits a mean Drug Liking Eₘₐₓ that is substantially lower by a margin of at least 9 when compared to 60 mg of phentermine hydrochloride per dose.

A composition comprising serdexmethylphenidate wherein the composition exhibits a lower mean Take Drug Again Eₘₐₓ when compared to phentermine following oral administration. The composition of this aspect, wherein the composition exhibits a substantially lower mean Take Drug Again Eₘₐₓ when compared to phentermine. The composition of this aspect, comprising an amount of serdexmethylphenidate, or a pharmaceutical salt thereof, per dose wherein the composition exhibits a substantially lower mean Take Drug Again Eₘₐₓ when compared to 60 mg of phentermine hydrochloride per dose. The composition of this aspect, wherein the serdexmethylphenidate is serdexmethylphenidate chloride and the amount of serdexmethylphenidate chloride is 120 mg per dose or less. The composition of this aspect, wherein the serdexmethylphenidate is serdexmethylphenidate chloride and the amount of serdexmethylphenidate chloride is 240 mg per dose or less. The composition of this aspect, wherein the serdexmethylphenidate is serdexmethylphenidate chloride and the amount of serdexmethylphenidate chloride is at least 120 mg per dose. The composition of this aspect, wherein the serdexmethylphenidate is serdexmethylphenidate chloride and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate, or a pharmaceutical salt thereof, wherein the composition exhibits a lower mean Overall Drug Liking Eₘₐₓ when compared to phentermine following oral administration. The composition of this aspect, wherein the composition exhibits a substantially lower mean Overall Drug Liking Eₘₐₓ when compared to phentermine. The composition of this aspect, comprising an amount of serdexmethylphenidate, or a pharmaceutical salt thereof, per dose wherein the composition exhibits a substantially lower mean Overall Drug Liking Eₘₐₓ when compared to 60 mg of phentermine hydrochloride per dose. The composition of this aspect, wherein the serdexmethylphenidate is serdexmethylphenidate chloride and the amount of serdexmethylphenidate chloride is 120 mg per dose or less. The composition of this aspect, wherein the serdexmethylphenidate is serdexmethylphenidate chloride and the amount of serdexmethylphenidate chloride is 240 mg per dose or less. The composition of this aspect, wherein the serdexmethylphenidate is serdexmethylphenidate chloride and the amount of serdexmethylphenidate chloride is at least 120 mg per dose. The composition of this aspect, wherein the serdexmethylphenidate is serdexmethylphenidate chloride and the amount of serdexmethylphenidate chloride is at least 240 mg per dose. The composition of this aspect, wherein the serdexmethylphenidate is serdexmethylphenidate chloride and the amount of serdexmethylphenidate chloride is about 120 mg to at least about 240 mg per dose.

A composition comprising serdexmethylphenidate wherein the composition exhibits a mean Drug Liking Eₘₐₓ that is significantly lower statistically than twice the maximum daily clinical dose of Focalin® XR following oral administration, wherein the maximum clinical daily dose is 40 mg.

A composition comprising serdexmethylphenidate wherein the composition exhibits a mean Drug Liking Eₘₐₓ that is significantly lower statistically following an oral dose of 120 mg of serdexmethylphenidate as compared to twice the maximum daily clinical dose of phentermine, wherein the maximum clinical daily dose is 30 mg.

A serdexmethylphenidate chloride composition that provides statistically significant reductions in maximal Drug Liking (Eₘₐₓ) at 120 mg and 240 mg of serdexmethylphenidate chloride when compared to Focalin® XR (80 mg) and at 120 mg serdexmethylphenidate chloride when compared to phentermine (60 mg) following oral administration.

A serdexmethylphenidate chloride composition that provides retrospective endpoints of Take Drug Again Eₘₐₓ and Overall Drug Liking Eₘₐₓ that are significantly lower for the serdexmethylphenidate chloride composition versus phentermine at both 120 mg and 240 mg doses of serdexmethylphenidate chloride following oral administration.

A serdexmethylphenidate chloride composition that provides Feeling High Eₘₐₓ and Good Effects Eₘₐₓ that are significantly reduced for both 120 mg and 240 mg doses of serdexmethylphenidate chloride when compared to Focalin® XR and phentermine hydrochloride following oral administration.

A method of orally administering an amount of serdexmethylphenidate chloride that results in abuse related effects that are lower compared to Focalin® XR. The method of this aspect, wherein the amount of serdexmethylphenidate chloride is up to 240 mg. The method of this aspect, wherein the abuse related effects are one or more of Drug Liking Eₘₐₓ, Feeling High Eₘₐₓ, Bad Effects Eₘₐₓ, or Good Effects Eₘₐₓ.

A method of orally administering an amount of serdexmethylphenidate chloride that results in abuse related effects that are lower compared to phentermine. The method of this aspect, wherein the amount of serdexmethylphenidate chloride is about 120 mg to about 240 mg. The method of this aspect, wherein the abuse related effects are one or more of Take Drug Again Eₘₐₓ, Overall Drug Liking Eₘₐₓ, Feeling High Eₘₐₓ, Bad Effects Eₘₐₓ, or Good Effects Eₘₐₓ.

A method of orally administering an amount of serdexmethylphenidate chloride that results in a Drug Liking Eₘₐₓ that is statistically lower than phentermine. The method of this aspect, wherein the amount of serdexmethylphenidate chloride is about 120 mg to about 240 mg.

A composition comprising serdexmethylphenidate, or a pharmaceutical salt thereof, wherein the composition has a dosage amount of serdexmethylphenidate chloride that provides a mean Take Drug Again Eₘₐₓ that is substantially similar to placebo following oral administration. The composition of this aspect, wherein the dosage amount is 120 mg or less. The composition of this aspect, wherein the dosage amount is 240 mg or less. The composition of this aspect, wherein the dosage amount is at least about 120 mg. The composition of this aspect, wherein the dosage amount is at least about 240 mg. The composition of this aspect, wherein the dosage amount is about 120 mg to at least about 240 mg.

A composition comprising serdexmethylphenidate, or a pharmaceutical salt thereof, wherein the composition has a dosage amount of serdexmethylphenidate chloride that provides a mean Overall Drug Liking Eₘₐₓ that is substantially similar to placebo following oral administration. The composition of this aspect, wherein the dosage amount is 120 mg or less. The composition of this aspect, wherein the dosage amount is 240 mg or less. The composition of this aspect, wherein the dosage amount is at least about 120 mg. The composition of this aspect, wherein the dosage amount is at least about 240 mg. The composition of this aspect, wherein the dosage amount is about 120 mg to at least about 240 mg.

Further aspects and embodiments of the present technology are described in the following paragraphs.

A composition comprising serdexmethylphenidate, or a salt thereof, wherein when the composition exhibits a lower mean Drug Liking Eₘₐₓ when compared to d-methylphenidate following intranasal administration of the composition to a human or animal subject. The composition of this aspect, wherein the composition exhibits a substantially lower mean Drug Liking Eₘₐₓ when compared to d-methylphenidate. The composition of this aspect, comprising an amount of serdexmethylphenidate, or a salt thereof, per dose wherein the composition exhibits a substantially lower mean Drug Liking Eₘₐₓ when compared to 40 mg of d-methylphenidate hydrochloride per dose following intranasal administration of the composition to a human or animal subject. The composition of this aspect, wherein the serdexmethylphenidate salt is serdexmethylphenidate chloride and the amount of serdexmethylphenidate chloride is 80 mg per dose or less. The composition of this aspect, wherein the serdexmethylphenidate salt is serdexmethylphenidate chloride and the amount of serdexmethylphenidate chloride is at least 80 mg per dose. The composition of this aspect, wherein the composition exhibits a mean Drug Liking Eₘₐₓ that is substantially lower by a margin of at least 10 when compared to 40 mg of d-methylphenidate hydrochloride per dose.

A serdexmethylphenidate chloride composition that provides statistically significant reductions in maximal Drug Liking Eₘₐₓ at 80 mg of serdexmethylphenidate chloride when compared to 40 mg d-methylphenidate hydrochloride following intranasal administration of the composition to a human or animal subject.

A serdexmethylphenidate composition that provides retrospective endpoints of Take Drug Again Eₘₐₓ and Overall Drug Liking Eₘₐₓ that are significantly lower for the serdexmethylphenidate composition when compared to 40 mg d-methylphenidate hydrochloride following intranasal administration of the composition to a human or animal subject. The composition of this aspect, wherein the serdexmethylphenidate is serdexmethylphenidate chloride and the amount of serdexmethylphenidate chloride is 80 mg per dose or less. The composition of this aspect, wherein the serdexmethylphenidate is serdexmethylphenidate chloride and the amount of serdexmethylphenidate chloride is at least 80 mg per dose.

A serdexmethylphenidate composition that provides a Feeling High Eₘₐₓ and a Good Effects Eₘₐₓ that are significantly reduced for the serdexmethylphenidate composition when compared to 40 mg d-methylphenidate hydrochloride following intranasal administration of the composition to a human or animal subject. The composition of this aspect, wherein the serdexmethylphenidate is serdexmethylphenidate chloride and the amount of serdexmethylphenidate chloride is 80 mg per dose or less. The composition of this aspect, wherein the serdexmethylphenidate is serdexmethylphenidate chloride and the amount of serdexmethylphenidate chloride is at least 80 mg per dose.

A serdexmethylphenidate composition that provides a Feeling Drowsy/Alert Eₘₐₓ that is significantly reduced for the serdexmethylphenidate composition when compared to 40 mg d-methylphenidate hydrochloride following intranasal administration of the composition to a human or animal subject. The composition of this aspect, wherein the serdexmethylphenidate is serdexmethylphenidate chloride and the amount of serdexmethylphenidate chloride is 80 mg per dose or less. The composition of this aspect, wherein the serdexmethylphenidate is serdexmethylphenidate chloride and the amount of serdexmethylphenidate chloride is at least 80 mg per dose.

A serdexmethylphenidate composition that provides an Any Effect Eₘₐₓ that is significantly reduced for the serdexmethylphenidate composition when compared to 40 mg d-methylphenidate hydrochloride following intranasal administration of the composition to a human or animal subject. The composition of this aspect, wherein the serdexmethylphenidate is serdexmethylphenidate chloride and the amount of serdexmethylphenidate chloride is 80 mg per dose or less. The composition of this aspect, wherein the serdexmethylphenidate is serdexmethylphenidate chloride and the amount of serdexmethylphenidate chloride is at least 80 mg per dose.

A serdexmethylphenidate composition that provides an Ease of Nasal Insufflation Eₘₐₓ that is significantly increased for the serdexmethylphenidate composition when compared to 40 mg d-methylphenidate hydrochloride following intranasal administration of the composition to a human or animal subject. The composition of this aspect, wherein the serdexmethylphenidate is serdexmethylphenidate chloride and the amount of serdexmethylphenidate chloride is 80 mg per dose or less. The composition of this aspect, wherein the serdexmethylphenidate is serdexmethylphenidate chloride and the amount of serdexmethylphenidate chloride is at least 80 mg per dose. The composition of this aspect, wherein the salt is a pharmaceutically acceptable salt.

A method of intranasal administration of an amount of serdexmethylphenidate that results in abuse related effects that are lower compared to d-methylphenidate. The method of this aspect, wherein the serdexmethylphenidate is serdexmethylphenidate chloride and the d-methylphenidate is d-methylphenidate hydrochloride. The method of this aspect, wherein the amount of serdexmethylphenidate chloride is 80 mg per dose or less. The method of this aspect, wherein the amount of serdexmethylphenidate chloride is at least 80 mg per dose. The method of this aspect, wherein the abuse related effects are one or more of Drug Liking Eₘₐₓ, Feeling High Eₘₐₓ, Feeling Drowsy/Alert Eₘₐₓ, or Good Effects Eₘₐₓ.

A method of intranasal administration of an amount of serdexmethylphenidate that results in abuse related effects that are not substantially different compared to a placebo. The method of this aspect, wherein the serdexmethylphenidate is serdexmethylphenidate chloride. The method of this aspect, wherein the amount of serdexmethylphenidate chloride is 80 mg per dose or less. The method of this aspect, wherein the amount of serdexmethylphenidate chloride is at least 80 mg per dose. The method of this aspect, wherein the abuse related effects are one or more of Take Drug Again Eₘₐₓ, Overall Drug Liking Eₘₐₓ, Feeling High Eₘₐₓ, Feeling Drowsy/Alert Eₘₐₓ, or Good Effects Eₘₐₓ. The method of this aspect, wherein the salt is a pharmaceutically acceptable salt.

A composition comprising serdexmethylphenidate, or a salt thereof, wherein the composition has a dosage amount of serdexmethylphenidate that provides a mean Take Drug Again Eₘₐₓ that is not substantially different to placebo following intranasal administration of the composition to a human or animal subject. The composition of this aspect, wherein the dosage amount is 80 mg or less. The composition of this aspect, wherein the dosage amount is at least about 80 mg.

A composition comprising serdexmethylphenidate, or a salt thereof, wherein the composition has a dosage amount of serdexmethylphenidate that provides a mean Overall Drug Liking Eₘₐₓ that is not substantially different to placebo following intranasal administration of the composition to a human or animal subject. The composition of this aspect, wherein the dosage amount is 80 mg or less. The composition of this aspect, wherein the dosage amount is at least about 80 mg. The composition of this aspect, wherein the salt is a pharmaceutically acceptable salt.

A composition comprising an amount of serdexmethylphenidate, or a salt thereof, that results in at least one improved abuse potential measure as compared to d-methylphenidate hydrochloride following intranasal administration of the composition to a human or animal subject. The composition of this aspect, wherein the amount of serdexmethylphenidate, or a salt thereof, results in at least two improved abuse potential measures. The composition of this aspect, wherein the amount of serdexmethylphenidate, or a salt thereof, results in at least three improved abuse potential measures. The composition of this aspect, wherein the amount of serdexmethylphenidate, or a salt thereof, results in at least four improved abuse potential measures. The composition of this aspect, wherein the improved abuse potential measure is a member selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

A composition comprising an amount of serdexmethylphenidate, or a salt thereof, that results in at least one abuse potential measure that is not substantially different as compared to a placebo following intranasal administration of the composition to a human or animal subject. The composition of this aspect, wherein the amount of serdexmethylphenidate, or a salt thereof, results in at least two abuse potential measures that are not substantially different as compared to a placebo. The composition of this aspect, wherein the amount of serdexmethylphenidate, or a salt thereof, results in at least three abuse potential measures that are not substantially different as compared to a placebo. The composition of this aspect, wherein the amount of serdexmethylphenidate, or a salt thereof, results in at least four abuse potential measures that are not substantially different as compared to a placebo. The composition of this aspect, wherein the not substantially different abuse potential measure is a member selected from the group consisting of Take Drug Again Eₘₐₓ and Overall Drug Liking Eₘₐₓ. The composition of this aspect, wherein the salt is a pharmaceutically acceptable salt.

A method of intranasal administration of an amount of serdexmethylphenidate chloride, or a salt thereof, that results in at least one improved abuse potential measure as compared to d-methylphenidate hydrochloride. The method of this aspect, wherein the administration of serdexmethylphenidate, or a pharmaceutically acceptable salt thereof, results in at least two improved abuse potential measures. The method of this aspect, wherein the administration of serdexmethylphenidate, or a pharmaceutically acceptable salt thereof, results in at least three improved abuse potential measures. The method of this aspect, wherein the administration of serdexmethylphenidate, or a pharmaceutically acceptable salt thereof, results in at least four improved abuse potential measures. The method of this aspect, wherein the administration of serdexmethylphenidate, or a pharmaceutically acceptable salt thereof, results in at least five improved abuse potential measures. The method of this aspect, wherein the improved abuse potential member is selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

A method of intranasal administration of an amount of serdexmethylphenidate chloride, or a salt thereof, that results in at least one abuse potential measure that is not substantially different as compared to placebo. The method of this aspect, wherein the administration of serdexmethylphenidate, or a pharmaceutically acceptable salt thereof, results in at least two abuse potential measures that are not substantially different as compared to a placebo. The method of this aspect, wherein the abuse potential measures comprise Take Drug Again Eₘₐₓ and/or Overall Drug Liking Eₘₐₓ. The method of this aspect, wherein the salt is a pharmaceutically acceptable salt.

A composition comprising an amount of serdexmethylphenidate, or a pharmaceutically acceptable salt thereof, that results in at least one abuse potential measure that is not substantially different as compared to placebo following intranasal administration of the composition to a human or animal subject. The composition of this aspect, wherein the composition that results in at least two abuse potential measures that are not substantially different as compared to placebo. The composition of this aspect, wherein the abuse potential measures comprise Take Drug Again Eₘₐₓ and/or Overall Drug Liking Eₘₐₓ. The composition of this aspect, wherein the salt is a pharmaceutically acceptable salt.

Further aspects and embodiments of the present technology are described in the following paragraphs.

A composition comprising serdexmethylphenidate, or a salt thereof, wherein when the composition exhibits a lower mean Drug Liking Eₘₐₓ when compared to d-methylphenidate following intravenous administration of the composition to a human or animal subject. The composition of this aspect, wherein the composition exhibits a substantially lower mean Drug Liking Eₘₐₓ when compared to d-methylphenidate. The composition of this aspect, comprising an amount of serdexmethylphenidate, or a salt thereof, per dose wherein the composition exhibits a substantially lower mean Drug Liking Eₘₐₓ when compared to 15 mg of d-methylphenidate hydrochloride per dose following intravenous administration of the composition to a human or animal subject. The composition of this aspect, wherein the serdexmethylphenidate salt is serdexmethylphenidate chloride and the amount of serdexmethylphenidate chloride is 30 mg per dose or less. The composition of this aspect, wherein the serdexmethylphenidate salt is serdexmethylphenidate chloride and the amount of serdexmethylphenidate chloride is at least 30 mg per dose. The composition of this aspect, wherein the composition exhibits a mean Drug Liking Eₘₐₓ that is substantially lower by a margin of at least 10 when compared to 15 mg of d-methylphenidate hydrochloride per dose.

A serdexmethylphenidate chloride composition that provides statistically significant reductions in maximal Drug Liking Eₘₐₓ at 30 mg of serdexmethylphenidate chloride when compared to 15 mg d-methylphenidate hydrochloride following intravenous administration of the composition to a human or animal subject.

A serdexmethylphenidate composition that provides retrospective endpoints of Take Drug Again Eₘₐₓ and Overall Drug Liking Eₘₐₓ that are significantly lower for the serdexmethylphenidate composition when compared to 15 mg d-methylphenidate hydrochloride following intravenous administration of the composition to a human or animal subject. The composition of this aspect, wherein the serdexmethylphenidate is serdexmethylphenidate chloride and the amount of serdexmethylphenidate chloride is 30 mg per dose or less. The composition of this aspect, wherein the serdexmethylphenidate is serdexmethylphenidate chloride and the amount of serdexmethylphenidate chloride is at least 30 mg per dose.

A serdexmethylphenidate composition that provides a Feeling High Eₘₐₓ and a Good Effects Eₘₐₓ that are significantly reduced for the serdexmethylphenidate composition when compared to 15 mg d-methylphenidate hydrochloride following intravenous administration of the composition to a human or animal subject. The composition of this aspect, wherein the serdexmethylphenidate is serdexmethylphenidate chloride and the amount of serdexmethylphenidate chloride is 30 mg per dose or less. The composition of this aspect, wherein the serdexmethylphenidate is serdexmethylphenidate chloride and the amount of serdexmethylphenidate chloride is at least 30 mg per dose.

A serdexmethylphenidate composition that provides a Feeling Drowsy/Alert Eₘₐₓ that is significantly reduced for the serdexmethylphenidate composition when compared to 15 mg d-methylphenidate hydrochloride following intravenous administration of the composition to a human or animal subject. The composition of this aspect, wherein the serdexmethylphenidate is serdexmethylphenidate chloride and the amount of serdexmethylphenidate chloride is 30 mg per dose or less. The composition of this aspect, wherein the serdexmethylphenidate is serdexmethylphenidate chloride and the amount of serdexmethylphenidate chloride is at least 30 mg per dose.

A serdexmethylphenidate composition that provides an Any Effect Eₘₐₓ that is significantly reduced for the serdexmethylphenidate composition when compared to 15 mg d-methylphenidate hydrochloride following intravenous administration of the composition to a human or animal subject. The composition of this aspect, wherein the serdexmethylphenidate is serdexmethylphenidate chloride and the amount of serdexmethylphenidate chloride is 30 mg per dose or less. The composition of this aspect, wherein the serdexmethylphenidate is serdexmethylphenidate chloride and the amount of serdexmethylphenidate chloride is at least 30 mg per dose.

A method of intravenous administration of an amount of serdexmethylphenidate that results in abuse related effects that are lower compared to d-methylphenidate. The method of this aspect, wherein the serdexmethylphenidate is serdexmethylphenidate chloride and the d-methylphenidate is d-methylphenidate hydrochloride. The method of this aspect, wherein the amount of serdexmethylphenidate chloride is 30 mg per dose or less. The method of this aspect, wherein the amount of serdexmethylphenidate chloride is at least 30 mg per dose. The method of this aspect, wherein the abuse related effects are one or more of Drug Liking Eₘₐₓ, Take Drug Again Eₘₐₓ, Feeling High Eₘₐₓ, Feeling Drowsy/Alert Eₘₐₓ, or Good Effects Eₘₐₓ.

A method of intravenous administration of an amount of serdexmethylphenidate that results in abuse related effects that are substantially similar compared to a placebo. The method of this aspect, wherein the serdexmethylphenidate is serdexmethylphenidate chloride. The method of this aspect, wherein the amount of serdexmethylphenidate chloride is 30 mg per dose or less. The method of this aspect, wherein the amount of serdexmethylphenidate chloride is at least 80 mg per dose. The method of this aspect, wherein the abuse related effects are one or more of Drug Liking Eₘₐₓ, Overall Drug Liking Eₘₐₓ, Feeling High Eₘₐₓ, Feeling Drowsy/Alert Eₘₐₓ, or Good Effects Eₘₐₓ. The method of this aspect, wherein the salt is a pharmaceutically acceptable salt.

A composition comprising serdexmethylphenidate, or a salt thereof, wherein the composition has a dosage amount of serdexmethylphenidate that provides a mean Take Drug Again Eₘₐₓ that is not substantially different to placebo following intravenous administration of the composition to a human or animal subject. The composition of this aspect, wherein the dosage amount is 30 mg or less. The composition of this aspect, wherein the dosage amount is at least about 30 mg.

A composition comprising serdexmethylphenidate, or a salt thereof, wherein the composition has a dosage amount of serdexmethylphenidate that provides a mean Overall Drug Liking Eₘₐₓ that is substantially similar to placebo following intravenous administration of the composition to a human or animal subject. The composition of this aspect, wherein the dosage amount is 30 mg or less. The composition of this aspect, wherein the dosage amount is at least about 30 mg. The composition of this aspect, wherein the salt is a pharmaceutically acceptable salt.

A composition comprising an amount of serdexmethylphenidate, or a salt thereof, that results in at least one improved abuse potential measure as compared to d-methylphenidate hydrochloride following intravenous administration of the composition to a human or animal subject. The composition of this aspect, wherein the amount of serdexmethylphenidate, or a salt thereof, results in at least two improved abuse potential measures. The composition of this aspect, wherein the amount of serdexmethylphenidate, or a salt thereof, results in at least three improved abuse potential measures. The composition of this aspect, wherein the amount of serdexmethylphenidate, or a salt thereof, results in at least four improved abuse potential measures. The composition of this aspect, wherein the improved abuse potential measure is a member selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

A composition comprising an amount of serdexmethylphenidate, or a salt thereof, that results in at least one abuse potential measure that is substantially similar as compared to a placebo following intravenous administration of the composition to a human or animal subject. The composition of this aspect, wherein the amount of serdexmethylphenidate, or a salt thereof, results in at least two abuse potential measures that are substantially similar as compared to a placebo. The composition of this aspect, wherein the amount of serdexmethylphenidate, or a salt thereof, results in at least three abuse potential measures that are substantially similar as compared to a placebo. The composition of this aspect, wherein the amount of serdexmethylphenidate, or a salt thereof, results in at least four abuse potential measures that are substantially similar as compared to a placebo. The composition of this aspect, wherein the substantially similar abuse potential measure is a member selected from the group consisting of Drug Liking Eₘₐₓ, Overall Drug Liking Eₘₐₓ, Feeling High Eₘₐₓ, and Good Effects Eₘₐₓ. The composition of this aspect, wherein the salt is a pharmaceutically acceptable salt.

A method of intravenous administration of an amount of serdexmethylphenidate chloride, or a salt thereof, that results in at least one improved abuse potential measure as compared to d-methylphenidate hydrochloride. The method of this aspect, wherein the administration of serdexmethylphenidate, or a pharmaceutically acceptable salt thereof, results in at least two improved abuse potential measures. The method of this aspect, wherein the administration of serdexmethylphenidate, or a pharmaceutically acceptable salt thereof, results in at least three improved abuse potential measures. The method of this aspect, wherein the administration of serdexmethylphenidate, or a pharmaceutically acceptable salt thereof, results in at least four improved abuse potential measures. The method of this aspect, wherein the administration of serdexmethylphenidate, or a pharmaceutically acceptable salt thereof, results in at least five improved abuse potential measures. The method of this aspect, wherein the improved abuse potential member is selected from the group consisting of Drug Liking Eₘₐₓ, Take Drug Again Eₘₐₓ, Overall Drug Liking Eₘₐₓ, Feeling High Eₘₐₓ, and Good Effects Eₘₐₓ.

A method of intravenous administration of an amount of serdexmethylphenidate chloride, or a salt thereof, that results in at least one abuse potential measure that is not substantially different as compared to placebo. The method of this aspect, wherein the abuse potential measure comprises Take Drug Again Eₘₐₓ. The method of this aspect, wherein the salt is a pharmaceutically acceptable salt.

A composition comprising an amount of serdexmethylphenidate, or a pharmaceutically acceptable salt thereof, that results in at least one abuse potential measure that is not substantially different as compared to placebo following intravenous administration of the composition to a human or animal subject. The composition of this aspect, wherein the abuse potential measure comprises Take Drug Again Eₘₐₓ. The composition of this aspect, wherein the salt is a pharmaceutically acceptable salt.

Further aspects and embodiments of the present technology are described in the following paragraphs.

A composition comprising serdexmethylphenidate, or a salt thereof, wherein, following oral administration in human or animal subjects, the composition results in d-methylphenidate exposure that can be scaled allometrically by body weight.

A composition comprising serdexmethylphenidate, or a salt thereof, and d-methylphenidate, or a salt thereof, wherein, following oral administration in human or animal subjects, the composition results in d-methylphenidate exposure that can be scaled allometrically by body weight. The composition of this aspect, wherein the d-methylphenidate exposure is adjusted for the dose of the composition. The composition of this aspect, wherein the d-methylphenidate exposure is measured by postdose plasma concentrations, Cₘₐₓ, AUC_{0-24 hr}, AUCₗₐₛₜ, or AUC_{inf}, or a combination thereof.

A composition comprising serdexmethylphenidate, or a salt thereof, wherein, following oral administration in human or animal subjects, the composition results in clearance (CL/F) of d-methylphenidate that can be scaled allometrically by body weight.

A composition comprising serdexmethylphenidate, or a salt thereof, and d-methylphenidate, or a salt thereof, wherein, following oral administration in human or animal subjects, the composition results in clearance (CL/F) of d-methylphenidate that can be scaled allometrically by body weight.

A composition comprising serdexmethylphenidate, or a salt thereof, wherein, following oral administration in human or animal subjects, the composition results in volume of distribution (V_{z}/F) of d-methylphenidate that can be scaled allometrically by body weight.

A composition comprising serdexmethylphenidate, or a salt thereof, and d-methylphenidate, or a salt thereof, wherein, following oral administration in human or animal subjects, the composition results in volume of distribution (V_{z}/F) of d-methylphenidate that can be scaled allometrically by body weight.

A composition comprising serdexmethylphenidate, or a salt thereof, and d-methylphenidate, or a salt thereof, wherein, following oral administration in a human or animal subject population, the 95% confidence interval of the geometric mean of the d-methylphenidate clearance is entirely contained in the interval of 60% to 140% of the geometric mean of the sample population.

A composition comprising serdexmethylphenidate, or a salt thereof, and d-methylphenidate, or a salt thereof, wherein, following oral administration in a human or animal subject population, the 95% confidence interval of the geometric mean of the d-methylphenidate volume of distribution is entirely contained in the interval of 60% to 140% of the geometric mean of the sample population. The composition of this aspect, wherein the sample population comprises at least 5 subjects. The composition of this aspect, wherein the sample population comprises no more than 10 subjects.

A composition comprising serdexmethylphenidate, or a salt thereof, wherein, following oral administration in human or animal subjects, the composition results in similar pharmacokinetic exposure parameters of d-methylphenidate between subjects when adjusted for dose and body weight.

A composition comprising serdexmethylphenidate, or a salt thereof, and d-methylphenidate, or a salt thereof, wherein, following oral administration in human or animal subjects, the composition results in similar pharmacokinetic exposure parameters of d-methylphenidate between subjects when adjusted for dose and body weight. The composition of this aspect, wherein the pharmacokinetic exposure parameters are plasma concentrations measured at the same time for each subject following oral administration of the composition. The composition of this aspect, wherein the pharmacokinetic exposure parameters are Cₘₐₓ, AUC_{0-24 hr}, AUCₗₐₛₜ, or AUC_{inf}, or a combination thereof.

A composition comprising serdexmethylphenidate, or a salt thereof, and d-methylphenidate, or a salt thereof, wherein, following oral administration in human or animal subjects, the composition results in similar clearance (CL/F) of d-methylphenidate between subjects when adjusted for body weight.

A composition comprising serdexmethylphenidate, or a salt thereof, and d-methylphenidate, or a salt thereof, wherein, following oral administration in human or animal subjects, the composition results in similar volume of distribution (VJF) of d-methylphenidate between subjects when adjusted for body weight.

A composition comprising serdexmethylphenidate, or a salt thereof, and d-methylphenidate, or a salt thereof, wherein, following oral administration in human or animal subjects, the composition results in similar Tₘₐₓ of d-methylphenidate. The composition of this aspect, wherein the human or animal subjects have different body weights. The composition of this aspect, wherein the human or animal subjects are of different ages. The composition of this aspect, wherein the human or animal subjects have different body weights and are of different ages. The composition of this aspect, wherein the human subjects are children, adolescents, or adults, or a combination thereof. The composition of this aspect, wherein the children are 2-12 years of age. The composition of this aspect, wherein the adolescents are 13-17 years of age. The composition of this aspect, wherein the adults are older than 17 years. The composition of this aspect, wherein the salt of serdexmethylphenidate is serdexmethylphenidate chloride. The composition of this aspect, wherein the salt of d-methylphenidate is d-methylphenidate hydrochloride. The composition of this aspect, wherein the total molar dose of the composition comprises about 90% serdexmethylphenidate and about 10% d-methylphenidate. The composition of this aspect, wherein the total molar dose of the composition comprises about 80% serdexmethylphenidate and about 20% d-methylphenidate. The composition of this aspect, wherein the total molar dose of the composition comprises about 70% serdexmethylphenidate and about 30% d-methylphenidate. The composition of this aspect, wherein the total molar dose of the composition comprises about 60% serdexmethylphenidate and about 40% d-methylphenidate. The composition of this aspect, wherein the total molar dose of the composition comprises about 50% serdexmethylphenidate and about 50% d-methylphenidate.

Further aspects and embodiments of the present technology are described in the following paragraphs.

A composition comprising serdexmethylphenidate, or a salt thereof, wherein the composition results in minimized and/or reduced adverse events in terms of severity, frequency, and/or duration when compared to unconjugated d-methylphenidate following intravenous administration to a human or animal subject. The composition of this aspect, comprising an amount of serdexmethylphenidate, or the pharmaceutical salt thereof, per dose, wherein the composition results in minimized and/or reduced adverse events in terms of severity, frequency, and/or duration when compared to 15 mg of d-methylphenidate hydrochloride per dose, following intravenous administration to a human or animal subject. The composition of this aspect, wherein the serdexmethylphenidate is serdexmethylphenidate chloride, and the amount of serdexmethylphenidate chloride is about 30 mg or less per dose. The composition of this aspect, wherein the serdexmethylphenidate is serdexmethylphenidate chloride, and the amount of serdexmethylphenidate chloride is at least about 30 mg per dose. The composition of this aspect, wherein the adverse events are minimized by being less severe when compared to unconjugated d-methylphenidate. The composition of this aspect, wherein the adverse events are reduced by being less frequent in terms of number of adverse events, number of subjects experiencing adverse events, or both, when compared to unconjugated d-methylphenidate. The composition of this aspect, wherein the adverse events are one or more of cardiac disorders, eye disorders, gastrointestinal disorders, general disorders and administration site conditions, investigations, nervous system disorders, psychiatric disorders, skin and subcutaneous disorders, or vascular disorders. The composition of this aspect, wherein the cardiac disorders are palpitations, tachycardia, sinus tachycardia, or a combination thereof. The composition of this aspect, wherein the gastrointestinal disorders are abdominal discomfort, dry mouth, nausea, or a combination thereof. The composition of this aspect, wherein the general disorders are asthenia, feeling abnormal, feeling cold, feeling hot, feeling jittery, feeling of relaxation, or a combination thereof. The composition of this aspect, wherein the site condition is heart rate increased. The composition of this aspect, wherein the nervous system disorders are dizziness, paraesthesia, somnolence, tremor, or a combination thereof. The composition of this aspect, wherein the psychiatric disorders are euphoric mood, hypervigilance, anxiety, bruxism, change in sustained attention, emotional disorder, insomnia, logorrhea, nightmare, or a combination thereof.

A composition comprising serdexmethylphenidate, or a salt thereof, wherein the composition results in minimized and/or reduced adverse events in terms of severity, frequency, and/or duration when compared to unconjugated d-methylphenidate following intranasal administration to a human or animal subject. The composition of this aspect, comprising an amount of serdexmethylphenidate, or the pharmaceutical salt thereof, per dose, wherein the composition results in minimized and/or reduced adverse events in terms of severity, frequency, and/or duration when compared to 40 mg of d-methylphenidate hydrochloride per dose, following intranasal administration to a human or animal subject. The composition of this aspect, wherein the serdexmethylphenidate is serdexmethylphenidate chloride, and the amount of serdexmethylphenidate chloride is 80 mg or less per dose. The composition of this aspect, wherein the serdexmethylphenidate is serdexmethylphenidate chloride, and the amount of serdexmethylphenidate chloride is at least 80 mg per dose. The composition of this aspect, wherein the adverse events are one or more of cardiac disorders, gastrointestinal disorders, general disorders and administration site conditions, investigations, nervous system disorders, psychiatric disorders, musculoskeletal and connective tissue disorders, skin and subcutaneous disorders, metabolism and nutrition disorders, or vascular disorders. The composition of this aspect, wherein the cardiac disorders are palpitations, tachycardia, sinus tachycardia, ventricular extrasystoles, or a combination thereof. The composition of this aspect, wherein the psychiatric disorders are euphoric mood, hypervigilance, anxiety, bruxism, restlessness, change in sustained attention, obsessive-compulsive disorder, phonophobia, or a combination thereof. The composition of this aspect, wherein the gastrointestinal disorder is dry mouth. The composition of this aspect, wherein the general disorders are fatigue, feeling hot, energy increased, chest discomfort, or a combination thereof. The composition of this aspect, wherein the investigation is blood pressure diastolic increased. The composition of this aspect, wherein the metabolism and nutrition disorder is decreased appetite. The composition of this aspect, wherein the musculoskeletal and connective tissue disorders are back pain, muscle tightness, muscle twitching, neck pain, or a combination thereof. The composition of this aspect, wherein the nervous system disorders are headache, somnolence, or a combination thereof. The composition of this aspect, wherein the vascular system disorder is flushing.

A composition comprising serdexmethylphenidate, or a salt thereof, wherein the composition results in certain increased adverse events when compared to unconjugated d-methylphenidate following intranasal administration to a human or animal subject. The composition of this aspect, comprising an amount of serdexmethylphenidate, or the pharmaceutical salt thereof, per dose, wherein the composition results in increased adverse events when compared to 40 mg of d-methylphenidate hydrochloride per dose, following intranasal administration to a human or animal subject. The composition of this aspect, wherein the serdexmethylphenidate is serdexmethylphenidate chloride, and the amount of serdexmethylphenidate chloride is 80 mg or less per dose. The composition of this aspect, wherein the serdexmethylphenidate is serdexmethylphenidate chloride, and the amount of serdexmethylphenidate chloride is at least 80 mg per dose. The composition of this aspect, wherein the adverse events are one or more of respiratory, thoracic and mediastinal disorders, and eye disorders. The composition of this aspect, wherein the respiratory, thoracic, and mediastinal disorders are nasal discomfort, nasal congestion, cough, rhinorrhoea, epistaxis, upper-airway cough syndrome, nasal dryness, sneezing, or a combination thereof. The composition of this aspect, wherein the eye disorders are lacrimation increased, eye pain, or a combination thereof.

The presently described technology is now described in such full, clear, concise and exact terms as to enable any person skilled in the art to which it pertains, to practice the same. It is to be understood that the foregoing describes preferred embodiments of the technology and that modifications may be made therein without departing from the spirit or scope of the invention.

It is to be understood that in some embodiments the term conjugate may encompass the terms compound and/or prodrug.

What is claimed:

1. A method of treating a patient having Attention Deficit Hyperactivity Disorder (ADHD), comprising orally administering to the patient a pharmaceutically effective amount of a composition comprising serdexmethylphenidate and dexmethylphenidate in an amount of 26 mg serdexmethylphenidate and 5.2 mg dexmethylphenidate.

2. The method of claim 1, wherein the serdexmethylphenidate has the following struscture

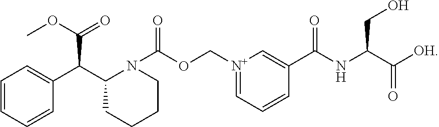 [SMILES: COC(=O)[C@H](c1ccccc1)[C@H]1CCCCN1C(=O)OC[n+]1cccc(C(=O)N[C@@H](CO)C(=O)O)c1]

3. The method of claim 1, wherein the serdexmethylphenidate is in the form of serdexmethylphenidate chloride, and wherein the 26 mg serdexmethylphenidate is equivalent to 28 mg serdexmethylphenidate chloride.

4. The method of claim 3, wherein the serdexmethylphenidate chloride has the following structure

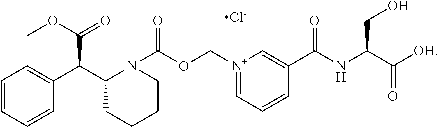 [SMILES: COC(=O)[C@H](c1ccccc1)[C@H]1CCCCN1C(=O)OC[n+]1cccc(C(=O)N[C@@H](CO)C(=O)O)c1 ; [Cl-]]

5. The method of claim 1, wherein the dexmethylphenidate is in the form of dexmethylphenidate hydrochloride, and wherein the 5.2 mg dexmethylphenidate is equivalent to 6 mg dexmethylphenidate hydrochloride.

6. The method of claim 1, wherein the combined molar does of serdexmethylphenidate and dexmethylphenidate is equivalent to 20 mg of dexmethylphenidate hydrochloride.

7. The method of claim 6, wherein the 20 mg of dexmethylphenidate hydrochloride is equivalent to 17.3 mg dexmethylphenidate free base.

8. The method of claim 1, wherein the composition is in the form of a capsule or tablet.

9. The method of claim 8, wherein the capsule is an immediate release capsule.

10. The method of claim 8, wherein the capsule or tablet is contained in a blister pack.

11. The method of claim 8, wherein the capsule or tablet is administered as a dosage of 1 capsule or tablet every day.

12. The method of claim 11, wherein the dosage is of 1 capsule or tablet taken in the morning.

13. The method of claim 8, wherein the dosage is administered with or without food.

14. The method of claim 13, wherein the dosage is administered with food.

15. The method of claim 8, wherein the dosage is in the form of a capsule and the capsule is opened and the composition of the capsule is sprinkled onto a tablespoon of a semi-solid or a liquid.

16. The method of claim 15, wherein the semi-solid is applesauce.

17. The method of claim 15, wherein the liquid is water.

18. A method of treating a patient having Attention Deficit Hyperactivity Disorder (ADHD), comprising orally administering to the patient a pharmaceutically effective amount of a composition comprising serdexmethylphenidate and dexmethylphenidate in an amount of 39 mg serdexmethylphenidate and 7.8 mg dexmethylphenidate.

19. The method of claim 18, wherein the serdexmethylphenidate has the following structure

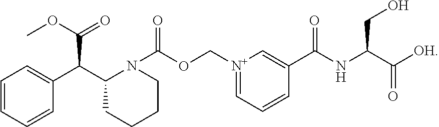 [SMILES: COC(=O)[C@H](c1ccccc1)[C@H]1CCCCN1C(=O)OC[n+]1cccc(C(=O)N[C@@H](CO)C(=O)O)c1]

20. The method of claim 18, wherein the serdexmethylphenidate is in the form of serdexmethylphenidate chloride, and wherein the 39 mg serdexmethylphenidate is equivalent to 42 mg serdexmethylphenidate chloride.

21. The method of claim 20, wherein the serdexmethylphenidate chloride has the following structure

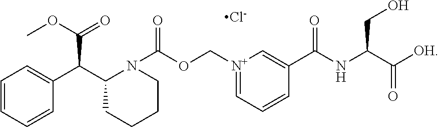 [SMILES: COC(=O)[C@H](c1ccccc1)[C@H]1CCCCN1C(=O)OC[n+]1cccc(C(=O)N[C@@H](CO)C(=O)O)c1 ; [Cl-]]

22. The method of claim 18, wherein the dexmethylphenidate is in the form of dexmethylphenidate hydrochloride, and wherein the 7.8 mg dexmethylphenidate is equivalent to 9 mg dexmethylphenidate hydrochloride.

23. The method of claim 18, wherein the combined molar does of serdexmethylphenidate and dexmethylphenidate is equivalent to 30 mg of dexmethylphenidate hydrochloride.

24. The method of claim 23, wherein the 30 mg of dexmethylphenidate hydrochloride is equivalent to 25.9 mg dexmethylphenidate free base.

25. The method of claim 18, wherein the composition is in the form of a capsule or tablet.

26. The method of claim 25, wherein the capsule is an immediate release capsule.

27. The method of claim 25, wherein the capsule or tablet is contained in a blister pack.

28. The method of claim 25, wherein the capsule or tablet is administered as a dosage of 1 capsule or tablet every day.

29. The method of claim 28, wherein the dosage is of 1 capsule or tablet taken in the morning.

30. The method of claim 25, wherein the dosage is administered with or without food.

31. The method of claim 30, wherein the dosage is administered with food.

32. The method of claim 25, wherein the dosage is in the form of a capsule and the capsule is opened and the composition of the capsule is sprinkled onto a tablespoon of a semi-solid or a liquid.

33. The method of claim 32, wherein the semi-solid is applesauce.

34. The method of claim 32, wherein the liquid is water.

35. A method of treating a patient having Attention Deficit Hyperactivity Disorder (ADHD), comprising orally administering to the patient a pharmaceutically effective amount of a composition comprising serdexmethylphenidate and dexmethylphenidate in an amount of 52 mg serdexmethylphenidate and 10.4 mg dexmethylphenidate.

36. The method of claim 35, wherein the serdexmethylphenidate has the following structure

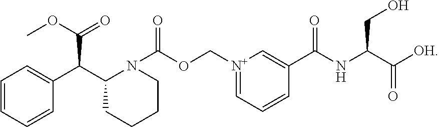 [SMILES: COC(=O)[C@H](c1ccccc1)[C@H]1CCCCN1C(=O)OC[n+]1cccc(C(=O)N[C@@H](CO)C(=O)O)c1]

37. The method of claim 35, wherein the serdexmethylphenidate is in the form of serdexmethylphenidate chloride, and wherein the 52 mg serdexmethylphenidate is equivalent to 56 mg serdexmethylphenidate chloride.

38. The method of claim 37, wherein the serdexmethylphenidate chloride has the following structure

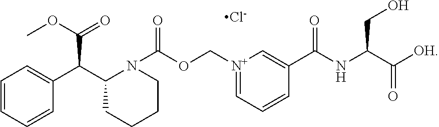 [SMILES: COC(=O)[C@H](c1ccccc1)[C@H]1CCCCN1C(=O)OC[n+]1cccc(C(=O)N[C@@H](CO)C(=O)O)c1 ; [Cl-]]

39. The method of claim 35 wherein the dexmethylphenidate is in the form of dexmethylphenidate hydrochloride, and wherein the 10.4 mg dexmethylphenidate is equivalent to 12 mg dexmethylphenidate hydrochloride.

40. The method of claim 35, wherein the combined molar does of serdexmethylphenidate and dexmethylphenidate is equivalent to 40 mg of dexmethylphenidate hydrochloride.

41. The method of claim 40, wherein the 40 mg of dexmethylphenidate hydrochloride is equivalent to 34.6 mg dexmethylphenidate free base.

42. The method of claim 35, wherein the composition is in the form of a capsule or tablet.

43. The method of claim 42, wherein the capsule is an immediate release capsule.

44. The method of claim 42, wherein the capsule or tablet is contained in a blister pack.

45. The method of claim 42, wherein the capsule or tablet is administered as a dosage of 1 capsule or tablet every day.

46. The method of claim 45, wherein the dosage is of 1 capsule or tablet taken in the morning.

47. The method of claim 42, wherein the dosage is administered with or without food.

48. The method of claim 47, wherein the dosage is administered with food.

49. The method of claim 42, wherein the dosage is in the form of a capsule and the capsule is opened and the composition of the capsule is sprinkled onto a tablespoon of a semi-solid or a liquid.

50. The method of claim 49, wherein the semi-solid is applesauce.

51. The method of claim 49, wherein the liquid is water.
